SEQUENCE LISTING (10 sequences)

SEQ ID NO: 1 - RNA, 74 nt, synthetic construct
  misc_feature 1..74: note = Secondary structure of the WT 5' BC1 domain
  source 1..74: mol_type = other RNA
ggggttggggatttagctcagtggtagagcgcttgcctagcaagcgcaaggccctgggttcggtcctcagctcc

SEQ ID NO: 2 - RNA, 74 nt, synthetic construct
  misc_feature 1..74: note = Secondary structure of the WC mutation of 5' BC1 domain
  source 1..74: mol_type = other RNA
ggggttggggatttagctcagtggtagagcgcttgcctagcaagcgctctgccctgggttcggtcctcagctcc

SEQ ID NO: 3 - RNA, 73 nt, synthetic construct
  misc_feature 1..73: note = Secondary structure of the mutant 5' BC1 deltaU22 domain
  source 1..73: mol_type = other RNA
ggggttggggatttagctcagggtagagcgcttgcctagcaagcgcaaggccctgggttcggtcctcagctcc

SEQ ID NO: 4 - RNA, 74 nt, synthetic construct
  misc_feature 1..74: note = Secondary structure of the mutant 5' BC1 deltaIL domain
  source 1..74: mol_type = other RNA
ggggttggggatttagctcagtggtagagcgcttgcctagcaagcgcaaggccctgggttaggtcctcagctcc

SEQ ID NO: 5 - RNA, 72 nt, synthetic construct
  misc_feature 1..72: note = Secondary structure of BC1
  source 1..72: mol_type = other RNA
gggttggggatttagctcagtggtagagcgcttgcctagcaagcgcaaggccctgggttcggtcctcagctc

SEQ ID NO: 6 - RNA, 69 nt, synthetic construct
  misc_feature 1..69: note = Secondary structure of BC200
  source 1..69: mol_type = other RNA
gaggcgggaggatagcttgagcccaggagttcgagacctgcctgggcaatatagcgagaccccgttctc

SEQ ID NO: 7 - RNA, 14 nt, synthetic construct
  misc_feature 1..14: note = BC200 RNA Decoy
  source 1..14: mol_type = other RNA
gcggcttgagcccg

SEQ ID NO: 8 - RNA, 16 nt, synthetic construct
  misc_feature 1..16: note = BC200 RNA Decoy
  source 1..16: mol_type = other RNA
cgggcaatatagccgc

SEQ ID NO: 9 - DNA, 19 bp, synthetic construct
  misc_feature 1..19: note = BC200 forward primer
  source 1..19: mol_type = other DNA
ggccgggcgcggtggctca

SEQ ID NO: 10 - DNA, 21 bp, synthetic construct
  misc_feature 1..21: note = BC200 reverse primer
  source 1..21: mol_type = other DNA
ggggggttgttgctttgaggg

(19) United States
(12) Patent Application Publication
(10) Pub. No.: US 2024/0336927 A1
(43) Pub. Date: Oct. 10, 2024

(54) BC200 RNA DECOY COMPOUNDS AND METHODS OF USE FOR THE TREATMENT OF AUTOIMMUNE DISEASES, INCLUDING LUPUS

(71) Applicant: The Research Foundation for the State University of New York, Albany, NY (US)

(72) Inventors: Henri Tiedge, Brooklyn, NY (US); Ilham Muslimov, Brooklyn, NY (US); Ellen Ginzler, Brooklyn, NY (US)

(21) Appl. No.: 18/293,099

(22) PCT Filed: Jul. 27, 2022
(86) PCT No.: PCT/US2022/074186

§ 371 Date: Jan. 29, 2024

Related U.S. Application Data
(60) Provisional application No. 63/227,124, filed on Jul. 29, 2021.

(51) Int. Cl. C12N 15/117

(52) U.S. Cl. CPC C12N 15/117; C12N 2310/13; C12N 2310/531; C12N 2310/533

(57) ABSTRACT

BC RNA decoys and methods of use thereof are disclosed which inhibit anti-BC antibodies from causing intraneuronal mislocalization of BC RNAs, thereby treating lupus.

### CROSS-REFERENCE TO RELATED APPLICATION

This application claims priority to U.S. Provisional Application No. 63/227,124, filed on Jul. 29, 2021, the entire disclosure of which is incorporated herein by reference as though set forth in full.

### GRANT SUPPORT STATEMENT

This invention was made with government support under Grant Numbers NS046769 and DA026110 awarded by the National Institutes of Health. The government has certain rights in the invention.

### INCORPORATION-BY-REFERENCE OF MATERIAL SUBMITTED IN ELECTRONIC FORM

The contents of the electronic sequence listing (SUNY-100-PCT.xml; Size: 14,254 bytes; and Date of Creation: Jul. 27, 2022) is herein incorporated by reference in its entirety.

### FIELD OF THE INVENTION

This invention relates to the fields of inhibitory nucleic acid molecules and autoimmune disease treatment. More specifically, the invention discloses administration of inhibitory nucleic acids, such as BC RNA decoys, in methods for inhibiting intraneuronal mislocalization of anti-BC antibodies, thereby ameliorating symptoms of neuropsychiatric lupus and other immune disorders.

### BACKGROUND OF THE INVENTION

Several publications and patent documents are cited throughout the specification in order to describe the state of the art to which this invention pertains. Each of these citations is incorporated herein by reference as though set forth in full.

Systemic lupus erythematosus (SLE) is an autoimmune disease in which neuropsychiatric involvement is common but poorly understood [1,2]. In neuropsychiatric lupus (NPSLE), a subtype of SLE, a CNS with a compromised blood brain barrier (BBB) is targeted by autoimmune antibodies [3], giving rise to neurological and psychiatric behavioral manifestations, prominent among them seizures and cognitive impairment [1,3-7]. A major gap in our understanding of NPSLE concerns the questions of how molecular autoimmune reactions give rise to the observed phenotypic alterations, and how such alterations can be targeted in therapeutic approaches.

SLE autoantibodies are often directed against nucleic acids or their binding proteins (Arbuckle et al., 2003; Elkon and Casali, 2008; Crow, 2010; Barrat et al., 2016). SLE anti-RNA autoantibodies typically target structured RNAs with double-stranded content (Schur and Monroe, 1969; Talal, 1973; Barrat et al., 2016). Examples of such autoantigens include ribosomal RNAs (rRNAs), e.g., 28S rRNA (Sturgill and Carpenter, 1965; Lamon and Bennett, 1970; Chu et al., 1991; Uchiumi et al., 1991; Elkon et al., 1992). Ribosomal RNAs form higher-order structural motifs, and it is mainly such motifs, rather than nucleotide sequence, that are recognized by ribosomal proteins (Noller, 2005; Grandin, 2010) and by autoimmune antibodies (Chu et al., 1991; Uchiumi et al., 1991). Similarly, higher-order structures in U1 spliceosomal RNA can trigger SLE autoimmune responses (van Venrooij et al., 1990; Hoet et al., 1999).

Regulatory brain cytoplasmic (BC) RNAs are non-protein-coding, small cytoplasmic RNAs (scRNAs) that, expressed in neurons, are located to synapto-dendritic domains (for review, see lacoangeli and Tiedge, 2013; Eom et al., 2018). BC RNAs control local protein synthesis by interacting with eukaryotic initiation factors (eIFs) 4A and 4B, thus repressing translation in the basal default state (Wang et al., 2002, 2005; Lin et al., 2008; Eom et al., 2011, 2014). After neuronal stimulation and receptor activation, translation is reversibly derepressed, effectively switching BC RNA translational control from a repressive to a permissive state (Eom et al., 2014). Responsible for translational control competence are C-loop architectural motifs in the BC RNA 3′ domains (Lin et al., 2008; Eom et al., 2011). BC RNA 5′ domains, in contrast, carry dendritic targeting elements (DTEs), spatial codes embedded in stem-loop noncanonical motif structures that specify targeted delivery to synapto-dendritic sites of function (Muslimov et al., 2006, 2011, 2018; Eom et al., 2018).

Control of mRNA translation, and thus of protein synthesis, in postsynaptic microdomains is a key mechanism in the activity-dependent regulation of local protein repertoires and consequently of synaptic form, function, and plasticity. While regulatory BC RNAs act as repressors in the basal default state, neuronal stimulation and receptor activation trigger a temporary switch from a repressive to a permissive state of translational control. Functional lack of BC RNA translational control causes neuronal hyperexcitability (manifesting in the form of prolonged epileptiform discharges and seizure activity) and cognitive dysfunction.

Clearly, a need exists in the art for preventing the targeting of SLE auto-antibodies.

### SUMMARY OF THE INVENTION

In accordance with the present invention, a composition for treating neuropsychiatric lupus comprising a BC RNA200 decoy of SEQ ID NO: 7, which inhibits SLE-anti-BC IgG mediated displacement of one or more of transport factors, hnRNP A2 and Purα, from BCRNA dendritic targeting elements is provided. In another embodiment, a composition for treating neuropsychiatric lupus comprising a BC RNA200 decoy of SEQ ID NO: 8, which also inhibits SLE-anti-BC IgG mediated displacement of one or more of transport factors, hnRNP A2 and Purα, from BCRNA dendritic targeting elements is disclosed. In certain embodiments, sequences can comprise a terminal phosphate at the 5′ end, a terminal phosphate at the 3′end or a terminal phosphate at both ends of said sequence, thereby inhibiting exonuclease degradation of said decoy sequence. In other embodiments, the sequences can comprise an additional 1, 2, 3, 4, 5, 6, 7, 8, 9, 10, 20 or 30 nucleotides at the 3′ end or the 5′end of said sequence, where the additional nucleotides being complementary to nucleotides present in the BCRNA200 region bound by said BC RNA200 decoy.

In yet another aspect, the sequences can comprise at least one modified nucleotide, wherein said modification is not present in nucleotides which hybridize to bulge region of the targeted BC200RNA. Such modifications include without limitation, one or more of 2-O-methyl, 2-O-methyoxy, LNA, FANA, UNA, a peptide nucleic acid, and an unnatural nucleotide. In certain aspects, at least one phosphodiester linkage is substituted with an internucleotide linkage selected from a phosphorothioate linkage, dithioate, 1-12C alkylphosphonate, methylphosphonate, amidate and triester. In preferred embodiments, the compositions are present in a cytoplasmic delivery vehicle. Suitable delivery vehicles include without limitation, liposomes, synthetic polymers, cell-penetrating peptides, nanoparticles, viral particles, electroporation buffers, and nucleofection reagents. In a preferred embodiment, the BC200 decoy is bound via a spacer to a nanoparticle which can be a lipid nanoparticle. In other embodiments, the BC200 RNA decoy sequence present in a vector. Suitable vectors include an adenoviral vector, an adeno-associated viral vector, a retroviral vector, and a lentiviral vector. The composition can also comprise at least one of an anti-inflammatory drug, an anticoagulation agent, and an immunosuppressive agent. In other embodiments, the composition is present in a pharmaceutically acceptable carrier.

Also within the scope of the invention is a method for treating neuropsychiatric lupus in a patient in need thereof comprising administration of an effective amount of the compositions described above to a patient in need thereof.

### BRIEF DESCRIPTION OF THE DRAWING

FIGS. 1A-1D. Secondary structures of WT and mutant 5′ BC1 domains. FIG. 1A (SEQ ID NO: 1), Secondary structure of the WT 5′ BC1 domain was established by chemical and enzymatic probing. GA motif, unpaired U22, and the basal internal loop (IL) are indicated. Noncanonical purine-purine pairs are symbolized by ⋅, standard WC pairs by =(GC) or −(AU), wobble WC pairs by. The noncanonical GA core motif resides in an A-form helix in the 5′ BC1 stem-loop domain. The GA core is clamped by canonical base pairs which are mostly G=C standard WC. The following 5′ domain mutations were introduced into full-length BC1 RNA. FIG. 1B (SEQ ID NO: 2), The WC mutation abolishes the 5′ BC1 GA motif by conversion to WC base pairing. FIG. 1C (SEQ ID NO:3), Mutant 5′ BC1 ΔU22 lacks the unpaired U residue at position 22. FIG. 1D (SEQ ID NO: 4), In mutant 5′ BC1 ΔIL, the basal internal loop has been eliminated by conversion to WC base pairing. Substitution mutations are indicated in red.

FIGS. 2A-2C. SLE autoimmune responses to BC RNAs. FIG. 2A, ³²P-labeled BC1 and BC200 RNAs were used in In EMSA analysis. RNAs bound in immune complexes were resolved from unbound RNAs by PAGE. BC1 and BC200 RNAs formed complexes with serum components from an SLE patient. FIG. 2B. The same serum was found nonreactive to BC200 DNA. Serum used in the experiments shown here was from SLE patient S1 (see below for patient codes). FIG. 2C, After antibody purification from serum(S) using Protein G, flow-through (FT) and three stepwise elution fractions (El1, El2, El3) were tested for anti-BC RNA reactivity by EMSA. While no reactivity was detected in FT, strong reactivity was apparent in E1 and E2 and weak reactivity was observed in E3. In this and the following figures, SLE autoantibodies are identified by patient codes in which a number may be preceded by the letters S (indicating an SLE patient with a history of seizures) or OBS (indicating an SLE patient with organic brain syndrome). SLE patients with no history of seizures or OBS are identified by numbers without preceding letters.

FIGS. 3A-3D. Anti-BC RNA reactivity of purified IgG preparations from sera of SLE patients, HS, and patients with non-SLE autoimmune diseases. P-labeled BC200 RNA was used for EMSA analysis. BC200 RNA in autoantibody-RNA complexes was separated from unbound BC200 RNA by PAGE. RNA only was loaded in the left-most lane of each of the four gels shown in FIG. 3A-3D. Preincubated RNA-antibody reaction mixtures were loaded in subsequent lanes of the gels FIG. 3A-3D. FIG. 3A, BC200 RNA was preincubated with IgG preparations from SLE patients. Anti-BC 200 RNA autoimmune reactivity was found varying among SLE IgG preparations between strong (lane 2 and 3, patients S1 and S6, respectively), weak (lanes 4-6, patients S13, S20, and OBS16, respectively), and nondetectable (lanes 7-13, patients 3, 15, S26, OBS18, 10, S16, and OBS15, respectively). Anti-BC 200 RNA reactivity was not detected in IgG preparations from subjects with no evidence of disease (HS, FIG. 3B, or in IgG preparations from patients with autoimmune diseases other than SLE, including RA (FIG. 3C), UC (FIG. 3C), or MS (FIG. 3D). For a positive reference (i.e., demonstration of autoantibody-RNA complexes) in gels FIG. 3B-3D, reaction mixtures of BC200 RNA preincubated with IgG SLE S1 were loaded in respective lanes 2. SLE autoantibody-BC RNA immune complexes often occur at two distinct electrophoretic mobilities: a strong band running at higher mobility (indicated as BC RNA auto-ab complexes), likely representing immune complexes containing one ab binding one RNA molecule, and a weaker band running at lower mobility, likely representing immune complexes containing one ab binding two RNA molecules. Electrophoretic mobility of BCRNA auto-ab complexes was lower with sera from SLE patients (FIG. 2) than with IgG preparations that were affinity-purified from such sera (this figure). It is possible that serum components interacting with immune complexes are responsible for this additional shift to lower mobility.

FIGS. 4A-4C. Molecular targets of SLE anti-BC autoimmune reactivity. EMSA experiments were performed with ³²P-labeled human BC200 RNA and rat BC1 RNA, using purified IgGs from SLE patient S6. FIG. 4A. Strong immune complex formation of IgG SLE S6 was apparent with BC200 RNA and BC1 RNA, with stronger reactivity toward the human than to the rat BC RNA. Immune complex formation was weak with spliceosomal U4 snRNA and undetectable with spliceosomal U6 snRNA. FIG. 4B, Robust reactivity was observed against full-length BC200 RNA and against the 5′ BC200 domain, but not against the 3′ BC200 domain. FIG. 4C, IgG SLE S6 formed immune complexes with full-length BC1 RNA and with its 5′ BC1 domain. When the 5′ DTE-resident GA motif was altered to standard A-form RNA helix by conversion of noncanonical base-pairings to standard WC base pairings (BC1 WC, see FIG. 1 for structure), full-length BC1 RNA was no longer recognized by IgG SLE S6. Similarly, IgG SLE S6 did not recognize full-length BC1 RNA in which 5′ DTE unpaired U22 had been deleted (BC1 ΔU22). By contrast, elimination of the 5′ basal internal loop (BC1 ΔIL) did not perceptibly alter BC1 RNA recognition by anti-BC IgG SLE S6. In each of the gels FIG. 4A-4C, RNA only was loaded in the left-most lane, and no complex formation was observed. While IgG SLE S6 was used in experiments shown in this figure, analogous results were obtained with three other IgG preparations purified from highly reactive SLE sera (Table 1).

FIGS. 5A-5B. EMSA competition analysis: transport factor hnRNP A2 is displaced from BC1 and BC200 RNAs by SLE anti-BC abs. FIG. 5A, ³²P-labeled BC1 RNA was loaded in lanes 1-6 as follows. Lane 1, unbound BC1 RNA runs with high mobility. Lane 2. BC1 RNA incubated with hnRNP A2 runs at reduced mobility, reflecting BC1 RNA-hnRNP A2 complex formation. Lane 3, BC1 RNA incubated with IgG from SLE patient S1 runs at reduced mobility, reflecting BC1 RNA-anti-BC antibody immune complex formation. Lane 4, BC1 RNA incubated with IgG from HS4 runs at the same mobility as unbound BC1 RNA in lane 1, indicating lack of immune complex formation. Lane 5, BC1 RNA incubated with hnRNP A2 and, after 30 min, with IgG from SLE patient S1 runs with the same mobility as BC1 RNA incubated with IgG from SLE patient S1; thus, dual incubation produced mostly complexes containing BC1 RNA and IgG SLE S1 rather than complexes containing BC1 RNA and hnRNP A2, indicating that hnRNP A2 had been displaced from BC1 RNA by IgG SLE S1. Lane 6, BC1 RNA incubated with hnRNP A2 and, after 30 min, with IgG from HS4 runs with the same mobility as BC1 RNA incubated with hnRNP A2; thus, dual incubation produced mostly complexes containing BC1 RNA and hnRNP A2 rather than complexes containing BC1 RNA and IgG HS4, indicating that IgG HS4 was unable to displace hnRNP A2 from BC1 RNA. Competition experiments with human BC200 RNA (lanes 7-12) were performed analogously. The molar ratio of IgG to hnRNP A2 was 1:1 in all cases. All final incubations were for 30 min. Analogous results were obtained when IgG SLE S6 was used instead of IgG SLE S1 (FIG. 6A). Unbound BC200 RNA runs at lower mobility than BC1 RNA because the former is longer (200 nt) than the latter (152 nt). FIG. 5B. Time course experiments reveal displacement, by IgG SLE S1, of hnRNP A2 from BC1 RNA (lanes 1-3) and from BC200 RNA (lanes 4-6). BC RNAs were incubated with hnRNP A2 and, after 30 min, with IgG SLE S1. Incubation was continued for 30 min (lanes 1 and 4), 45 min (lanes 2 and 5), and 60 min (lanes 3 and 6). The data indicate that hnRNP A2 was completely displaced from BC1 RNA and BC200 RNA by IgG SLE S1 within 60 min.

FIGS. 6A-6B. EMSA competition analysis: displacement of transport factor hnRNP A2 by anti-BC abs IgG SLE S1 and IgG SLE S6. FIG. 6A, Displacement of hnRNP A2 from BC1 RNA by IgG SLE S1 and IgG SLE S6. Electrophoretic mobility levels are indicated to the left of the autoradiogram as follows: mobility level I. BC1 RNA-IgG complexes; mobility level II, BC1 RNA-hnRNP A2 complexes; mobility level III, unbound BC1 RNA. P-labeled BC1 RNA was loaded in lanes 1-12 as follows. Lane 1, unbound BC1 RNA runs with high mobility. Lane 2, BC1 RNA incubated with hnRNP A2; reduced mobility indicates BC1 RNA-hnRNP A2 complex formation. Lane 3, BC1 RNA incubated with IgG SLE S1; reduced mobility indicates BC1 RNA-anti-BC antibody immune complex formation. Lane 4, BC1 RNA incubated with IgG SLE S6; reduced mobility indicates BC1 RNA-anti-BC antibody immune complex formation. Lane 5, BC1 RNA incubated with IgG HS4; same mobility as that of unbound BC1 RNA in lane 1 indicates lack of immune complex formation. Lane 6, BC1 RNA incubated with hnRNP A2 and, after 30 min, with IgG SLE S1; data indicate displacement of hnRNP A2 from BC1 RNA by IgG SLE S1. Lane 7, BC1 RNA incubated with hnRNP A2 and, after 30 min, with IgG SLE S6; data indicate displacement of hnRNP A2 from BC1 RNA by IgG SLE S6. Lane 8, BC1 RNA incubated with hnRNP A2 and, after 30 min, with IgG HS4; data indicate no displacement of hnRNP A2 from BC1 RNA by IgG HS4. Molar ratio of IgG to hnRNP A2 was 1:1. All final incubations were for 30 min. Lanes 10-12: BC1 RNA was incubated with hnRNP A2 and, after 30 min, with IgG SLE S6. Incubation was continued for the times indicated. The mobility shifts indicate complete displacement of hnRNP A2 from BC1 RNA by IgG SLE S6 within 60 min. FIG. 6B, Differential BC RNA complex formation with autoimmune IgGs. EMSA analysis was performed with 32P-labeled BC200 RNA, using IgG SLE S1 and IgG SLE S6 (SLE patients), IgG HS4. IgG MS1 (MS patient), and IgG RA1 (RA patient). Shifts to lower mobility occur with IgG SLE S1 and IgG SLE S6, but not with IgG HS4. IgG MS1, or IgG RA1, indicating that only SLE IgGs but not IgGs from HS or from non-SLE autoimmune disease patients react with BC200 RNA to form immune complexes.

FIGS. 7A-7G. Dendritic transport of rat BC1 RNA: impairment after coinjection with SLE anti-BC IgGs. After preincubation with IgGs, radiolabeled BC1 RNA was coinjected into sympathetic neurons in primary culture. Coinjection was performed with IgG HS4 (FIG. 7A, 7B). IgG SLE S1 (FIG. 7C, 7D), or IgG MS1 (FIG. 7E, 7F). In dark-field photomicrographs (FIG. 7A, 7C, 7E), labeling signal appears as white silver grains. Typical neuronal morphology and dendritic extent of sympathetic neurons in culture (Muslimov et al., 2011, 2014) are observed in phase-contrast photomicrographs FIG. 7B (corresponding to dark-field photomicrograph FIG. 7A). FIG. 7D (corresponding to dark-field photomicrograph FIG. 7C), and FIG. 7F (corresponding to dark field photomicrograph E). Scale bar. 50 μm. Numbers of cells analyzed: FIG. 7A, 7B. 7 neurons. 28 dendrites; FIG. 7C, 7D. 8 neurons. 28 dendrites; and FIG. 7E, 7F. 7 neurons. 28 dendrites. Quantitative analysis (FIG. 7G) indicates that IgG SLE S1, but not IgG HS4 or IgG MS1, significantly reduces dendritic delivery of BC1 RNA.

FIGS. 8A-8G. Dendritic transport of human BC200 RNA: impairment after coinjection with SLE anti-BC IgGs. Coinjection was performed, as described for FIG. 7, with IgG HS4 (FIG. 8A, 8B). IgG SLE S1 (FIG. 8C, 8D), or IgG MS1 (FIG. 8E, 8F). Dark-field photomicrographs (FIG. 8A, 8C, 8E) are accompanied by corresponding phase-contrast photomicrographs (FIG. 8B, 8D, 8F). Scale bar. 50 μm. Number of cells analyzed: FIG. 8A, 8B. 9 neurons. 33 dendrites; FIG. 8C, 8D. 8 neurons. 28 dendrites; and FIG. 8E, 8F. 7 neurons. 27 dendrites. Quantitative analysis (FIG. 8G) indicates that IgG SLE S1, but not IgG HS4 or IgG MS1, significantly reduces dendritic delivery of BC200 RNA.

FIGS. 9A-9G. Dendritic transport of rat BC1 RNA: impairment after bath application of SLE anti-BC IgGs. Sympathetic neurons were microinjected with radiolabeled BC1 RNA after bath application of IgGs for 8 h. Bath application was performed with IgG HS4 (FIG. 9A, 9B), IgG SLE S6 (FIG. 9C, 9D), or IgG RA1 (FIG. 9E, 9F). Dark-field photomicrographs (FIG. 9A, 9C, 9E) are accompanied by corresponding phase-contrast photomicrograph (FIG. 9B), DIC Nomarski photomicrograph (FIG. 9D), and phase-contrast photomicrograph (FIG. 9F). Scale bar. 50 μm. Number of cells analyzed: FIG. 9A, 9B. 8 neurons. 28 dendrites; FIG. 9C, 9D. 7 neurons. 28 dendrites; and E. F. 8 neurons. 27 dendrites. Quantitative analysis (FIG. 9G) indicates that bath-applied IgG SLE S6, but not bath-applied IgG HS4 or IgG RA1, significantly reduces dendritic delivery of BC1 RNA.

FIGS. 10A-10G. Dendritic transport of human BC200RNA: impairment after bath application of SLE anti-BCIgGs. Bath application was performed with IgGHS4 (FIG. 10A, 10B). IgGSLES1 (FIG. 10C, 10D), or IgGMS1 (FIG. 10E, 10F). Dark-field photomicrographs (FIG. 10A, 10C, 10E) are accompanied by phase-contrast (FIG. 10B) and DIC Nomarski (FIG. 10D, 10F) photomicrographs. Scale bar. 50 μm. Number of cells analyzed: FIG. 10A, 10B. 8 neurons. 32 dendrites; FIG. 10C, 10D. 7 neurons. 28 dendrites; FIG. 10E, 10F. 7 neurons, 27 dendrites. Quantitative analysis (FIG. 10G) indicates that bath-applied IgG SLE S1, but not bath-applied IgG HS4 or IgG MS1, significantly reduces dendritic delivery of BC200 RNA.

FIGS. 11A-11F. SLE anti-BC abs: mislocalization of endogenous BC1 RNA in vivo. Mice were injected intravenously with SLE anti-BC abs or with non-SLE abs and, subsequently, intraperitoneally with LPS to permeabilize the BBB. The following antibodies were used: SLE S1, SLE S6, HS4, MS1, and RA. O. Stratum oriens; P, stratum pyramidale; R, stratum radiatum; hf, hippocampal fissure. FIG. 11A-11D, Hippocampal CA1 regions (coronal sections) of injected animals are shown in dark-field (FIG. 11A, 11C) and corresponding bright-field (FIG. 11B, 11D) photomicrographs. Scale bar. 100 μm. BC1 RNA labeling signal is apparent as white silver grains, resulting from emulsion autoradiography, in dark-field photomicrographs. FIG. 11A, 11B. CA1 region of an animal injected with IgG HS4 (i.e., from an HS). BC1 RNA labeling signal is robust in dendritic layers stratum oriens and stratum radiatum. FIG. 11C, 11D. CA1 region of an animal injected with IgG SLE S1 (i.e., from an SLE patient with seizures). BC1 RNA labeling signal is prominent in somatic layer stratum pyramidale. FIG. 11E, 11F. Quantitative analysis. Autoradiographic signal intensities were established in stratum pyramidale (center), in stratum oriens at a distance of 50 μm from the edge of stratum pyramidale, and in stratum radiatum at distances of 50, 100, and 150 μm from the edge of stratum pyramidale. FIG. 11E. Animals injected with non-SLE abs (10 animals). FIG. 11F. Animals injected with SLE anti-BC abs (n=12 animals).

FIG. 12. SLE anti-BC abs in neurons. Delivery of BCRNAs to synapto-dendritic domains requires transport factor hnRNPA2. The factor interacts with targeting-determinant GA motifs contained within BC RNA 5′ stem-loop domains. SLE anti-BC IgGs bind to the same motif structures, displacing hnRNP A2 from BC RNAs and as a result causing impaired dendritic delivery.

FIG. 13. Secondary structures of BC1 (SEQ ID NO: 5) and BC200 (SEQ ID NO: 6) 5′ stem-loop domains. Noncanonical pairs are symbolized by ⋅, standard WC pairs by =(GC) or −(AU), wobble WC pairs by. Noncanonical GA core motifs reside in A-form helices that are part of the 5′ stem-loop domains. GA cores are clamped by canonical base pairs which are mostly of the GC standard WC type. Unpaired U nucleotides reside adjacent to GA cores. Noncanonical base pairs (e.g. G⋅A) are strong, comparable in stability to standard WC pairs.

FIGS. 14A-14B. BC1 and BC200 RNA: structural motif recognition. (FIG. 14A) In EMSA, purified IgG SLE S6 reacted with full-length BC1 RNA, with the BC1 5′ domain, and with full-length BC1 RNA in which the basal internal loop has been converted to standard WC base pairing (BC1ΔIL). In contrast, little or no reactivity was observed with BC1 RNA in which the non-canonical base pairs in the GA motif have been converted to standard WC pairing (BC1AGA), or with BC1 RNA in which unpaired U22 has been eliminated (BC1ΔU22). Unbound RNA was allowed to run off the gel. (FIG. 14B) BC RNA transport factors hnRNP A2 and Purα, as well as IgG SLE S6, interacted with BC200 RNA via the apical GA motif (GA1) in the 5′ DTE. Conversion of GA1 to WC base pairing (GA1 Mutant) abolished binding. Analogous conversion of the basal GA motif (GA2 Mutant) had no effect. Concurrent mutation of GA1 and GA2 resulted in abolished binding.

FIG. 15. Minimal structure for BC200 Decoys (SEQ ID NOs: 7 and 8) shown for RNA Oligos in descending order.

FIGS. 16A-16B. Displacement of hnRNP A2 from BC1 and BC200 RNAs by SLE anti-BC abs. (FIG. 16A) BC1 RNA was incubated with hnRNP A2 (lane 2), with IgG from SLE patient S1 (lane 3), with IgG from healthy subject 4 (IgG HS4, lane 4), with hnRNP A2 and, after 30 min, with IgG SLE S1 (lane 5), with hnRNP A2 and, after 30 min, with IgG HS4 (lane 6). Experiments with BC200 RNA were performed analogously. The molar ratio of IgG to hnRNP A2 was 1:1. All final incubations were for 30 min. IgGs from RA or MS patients did not produce mobility shifts with BC200 RNA or BC1 RNA. (FIG. 16B) BC1 RNA (left) and BC200 RNA (right) were incubated with hnRNP A2 and, after 30 min, with IgG SLE S1. Incubation was continued for the time periods indicated (in min).

### DETAILED DESCRIPTION OF THE INVENTION

The role of regulatory Brain-specific Cytoplasmic RNAs (BC RNAs) in the control of local protein synthesis at neuronal synapses has been previously reviewed [8-10]. Instrumental in the implementation of neuronal plasticity, this mechanism utilizes the functionalities of noncanonical BC RNA architectural motifs. These functionalities are twofold. (i) A 3′ C-loop motif is responsible for translational control (repressive in the basal default state, permissive following receptor activation) by interacting with translation initiation factors [11]. (ii) A 5′ GA motif operates as a dendritic targeting element (DTE) as it mediates BC RNA transport to sites of function in postsynaptic microdomains. In model animals, lack of rodent BC1 RNA, either cell-wide or locally at the synapse, gives rise to translational dysregulation with consequential phenotypic alterations that include seizures and cognitive dysfunction [12-15].

We discovered that autoantibodies from a subset of SLE patients are directed against neuroregulatory BC RNAs (SLE anti-BC abs). High-reactivity anti-BC abs were detected in 26% of serum samples obtained from lupus patients at SUNY. These SLE anti-BC abs engage the 5′ GA motif that serves as a dendritic targeting element (DTE) [16]. SLE anti-BC abs quasi permanently displace RNA transport factors from the GA motif DTE and thereby prevent BC RNA delivery to synapto-dendritic sites of function [16]. The data indicate that neuronal regulatory BC RNAs can elicit autoimmune responses that lead to phenotypic manifestations in neuropsychiatric SLE. We predict that such manifestations will be analogous to those resulting from cell wide or local synaptic lack of BC1 RNA in model animals, including seizures and cognitive impairment.

Here, we report that the architectural motif structures in regulatory BC RNAs are targets of autoimmune reactivity in SLE. We detected anti-BC RNA reactivity in sera of a subset of SLE patients, and we determined that SLE anti-BC RNA auto-antibodies (anti-BC abs) are directed against DTE motif structures in 5′ stem-loop domains. Anti-BC abs effectively compete with RNA transport factor heterogeneous nuclear ribonucleoprotein A2 (hnRNP A2) for access to these structures. After up-take by neurons in primary culture or CNS neurons in vivo, SLE anti-BC abs significantly diminish BC RNA delivery to synapto-dendritic domains. Absence of BC1 RNA in the BC1 knock-out (KO) animal model causes epileptogenic susceptibility (Zhong et al., 2009, 2010) and cognitive dysfunction (Briz et al., 2017; Chung et al., 2017; lacoangeli et al., 2017).

### Definitions

The present subject matter may be understood more readily by reference to the following detailed description which forms part of this disclosure. It is to be understood that this invention is not limited to the specific products, methods, conditions or parameters described and/or shown herein, and that the terminology used herein for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, cd. (1987)).

As employed above and throughout the disclosure, the following terms and abbreviations, unless otherwise indicated, shall be understood to have the following meanings.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

A “sample” refers to a sample from a subject that may be tested. The sample may comprise cells, and it may comprise body fluids, such as blood, serum, plasma, cerebral spinal fluid, urine, saliva, tears, pleural fluid, and the like. The sample may also be a tissue sample, or cells derived from a tissue.

As used herein, the terms “component,” “composition,” “composition of compounds,” “compound,” “drug.” “pharmacologically active agent,” “active agent.” “therapeutic,” “therapy,” “treatment.” or “medicament” are used interchangeably herein to refer to a compound or compounds or composition of matter, such as a chemical compound, a mixture of chemical compounds, a biological macromolecule, or an extract made from biological materials such as bacteria, plants, fungi, or animal (particularly mammalian) cells or tissues, which, when administered to a subject (human or animal) induces a desired pharmacological and/or physiologic effect by local and/or systemic action.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

A cell can be in vitro. Alternatively, a cell can be in vivo and can be found in a subject. A “cell” can be a cell from any organism including, but not limited to, a bacterium.

By the term “effective amount” of a compound as provided herein is meant a nontoxic but sufficient amount of the compound to provide the desired result.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

The terms “polynucleotide”, “nucleotide”, “nucleotide sequence”, “nucleic acid”, and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Upstream” and “Downstream” respectively refer to moving along a nucleotide strand in a 3′ to 5′ direction or a 5′ to 3′ direction.

“Introducing into” means uptake or absorption in the cell, as is understood by those skilled in the art. Absorption or uptake of oligos can occur through cellular processes, or via the use of auxiliary agents or devices.

The term “identity” as used herein and as known in the art, is the relationship between two or more oligo sequences, and is determined by comparing the sequences. Identity also means the degree of sequence relatedness between oligo sequences, as determined by the match between strings of such sequences. Identity can be readily calculated (see, e.g., Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While a number of methods to measure identity between two polynucleotide sequences are available, the term is well known to skilled artisans (see, e.g., Sequence Analysis in Molecular Biology, von Heinje, G., Academic Press (1987); and Sequence Analysis Primer, Gribskovm, M. and Devereux, J., eds., M. Stockton Press, New York (1991)). Methods commonly employed to determine identity between oligo sequences include, for example, those disclosed in Carillo, H., and Lipman, D., Siam J. Applied Math. (1988) 48:1073. In certain embodiments, the present invention may have 75, 80, 85, 90, 91, 92, 93, 94, 95, 96, 97, 98, 99, or 100% identity with the SEQ ID NOs disclosed herein.

“Substantially identical,” as used herein, means there is a very high degree of homology preferably >90% sequence identity.

The term “exogenous” nucleic acid can refer to a nucleic acid that is not normally or naturally found in or produced by a given bacterium, organism, or cell in nature. The term “endogenous” nucleic acid can refer to a nucleic acid that is normally found in or produced by a given bacterium, organism, or cell in nature.

The term “recombinant” is understood to mean that a particular nucleic acid (DNA or RNA) or protein is the product of various combinations of cloning, restriction, or ligation steps resulting in a construct having a structural coding or non-coding sequence distinguishable from endogenous nucleic acids found in natural systems.

“Oligonucleotides” are single stranded oligonucleotides or “oligos” that inhibit the expression or function of the target by interfering with some step in the sequence of events leading to gene expression and/or subsequent protein production or function by directly interfering with the step. Oligonucleotides can be decoy RNA molecules, siRNA, shRNA, and antisense oligonucleotides.

Other oligos act by inducing gene target transcript digestion. BC200 decoy RNAs inhibit binding of pathogenic autologous antibodies to BC200 RNA, thereby ameliorating autoimmune disease symptoms, particularly symptoms of neuropsychiatric lupus and cognitive impairment.

“Antisense oligos or strands” are oligos that are complementary to sense oligos, pre-mRNA, RNA or sense strands of particular genes and which bind to such genes and gene products by means of base pairing. When binding to a sense oligo, the antisense oligo need not base pair with every nucleoside in the sense oligo. All that is necessary is that there be sufficient binding to provide for a Tm of greater than or equal to 40° C. under physiologic salt conditions at sub-micromolar oligo concentrations.

As used herein, “antisense activity” means any detectable and/or measurable change attributable to the hybridization of an antisense compound to its target nucleic acid.

The term “lipid nanoparticle” refers to particles having at least one dimension on the order of nanometers (e.g., 1-1,000 nm) which include one or more of the compounds of structure (I) or other specified cationic lipids. In some embodiments, lipid nanoparticles are included in a formulation that can be used to deliver an active agent or therapeutic agent, such as a nucleic acid (e.g., decoy RNA) to a target site of interest (e.g., cell, tissue, organ, tumor, and the like). In some embodiments, the lipid nanoparticles of the invention comprise a nucleic acid. Such lipid nanoparticles typically comprise a compound having the structure of Formula I described in U.S. Pat. No. 11,040,212 (incorporated herein by reference) and one or more excipient selected from neutral lipids, charged lipids, steroids and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm. 145 nm, or 150 nm, and are substantially non-toxic. In certain embodiments, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058; WO 2013/086373; U.S. Pat. No. 10,987,308; 10;092,617; and 10,046,057 the full disclosures of which are herein incorporated by reference in their entirety for all purposes.

As used herein, “lipid encapsulated” refers to a lipid nanoparticle that provides an active agent or therapeutic agent, such as a nucleic acid (e.g., decoy RNA), with full encapsulation, partial encapsulation, or both. In an embodiment, the nucleic acid (e.g., decoy RNA) is fully encapsulated in the lipid nanoparticle.

As used herein, the term “aqueous solution” refers to a composition comprising water.

“Serum-stable” in relation to nucleic acid-lipid nanoparticles means that the nucleotide is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of a therapeutic product that can result in a broad exposure of an active agent within an organism. Some techniques of administration can lead to the systemic delivery of certain agents, but not others. Systemic delivery means that a useful, preferably therapeutic, amount of an agent is exposed to most parts of the body. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery.

“Local delivery.” as used herein, refers to delivery of an active agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous or intradermal injection. Local delivery does not preclude a systemic pharmacological effect.

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measurable activity” means a measurable activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenylation, addition of 5′-cap), translation, and post-translational modification.

As used herein, “translation” means the process in which a polypeptide (e.g. a protein) is translated from an mRNA. In certain embodiments, an increase in translation means an increase in the number of polypeptide (e.g. a protein) molecules that are made per copy of mRNA that encodes said polypeptide.

As used herein, “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions.

As used herein, “mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides.

The terms “construct”, “cassette”, “expression cassette”, “plasmid”, “vector”, or “expression vector” is understood to mean a recombinant nucleic acid, generally recombinant DNA, which has been generated for the purpose of the expression or propagation of a nucleotide sequence(s) of interest, or is to be used in the construction of other recombinant nucleotide sequences.

The term “promoter” or “promoter polynucleotide” is understood to mean a regulatory sequence/element or control sequence/element that is capable of binding/recruiting an RNA polymerase and initiating transcription of sequence downstream or in a 3′ direction from the promoter. A promoter can be, for example, constitutively active, or always on, or inducible in which the promoter is active or inactive in the presence of an external stimulus. Example of promoters include T7 promoters or U6 promoters.

The term “operably linked” can mean the positioning of components in a relationship which permits them to function in their intended manner. For example, a promoter can be linked to a polynucleotide sequence to induce transcription of the polynucleotide sequence.

The terms “complementarity” or “complement” refer to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%. 98%, 99%, or 100%, or percentages in between over a region of 4, 5, 6, 7, and 8 nucleotides, or refers to two nucleic acids that hybridize under stringent conditions.

A nucleic acid as described herein can be “modified” to increase stability in vivo. Such modifications include, without limitation, sugar modifications such as 2′fluoro, 2′-O-methyl, 2′-NH2. The phosphodiester backbone linkage can also be substituted with phosphorothioate as disclosed herein, but other backbone modifications such as triazole linked, or phNA are known to the skilled artisan. Additionally, modified bases can be employed, including without limitation, 7-deaza-dA, and carboxamide-dU.

LNA modification consists of the introduction of a 2′-O,4′-C-methylene bridge in the sugar ring, locking the ring in a stable furanose conformation. This modification improves RNA duplex stability, decreasing the susceptibility to nuclease degradation. Multiple LNA modifications may decrease decoy RNA efficacy but, if appropriately used, these are the most efficient in increasing target binding affinity. LNA modification raises the duplex melting temperature to complementary RNA from 2 to 8 degrees per introduced LNA. The high melting temperatures conferred by LNA allow the use of truncated decoys (15-mer down to 8-mer). Tiny 8-mer LNA-modified anti-miRs targeting the miRNA seed region have been used successfully to inhibit a whole family of miRNAs. Because miRNAs from the same family often act redundantly and synergistically in activating or inhibiting a determined cellular process, the ability of tiny LNA to inhibit a whole family of miRNAs at the same time represents a valuable therapeutic approach.

Unlocked nucleic acids (UNA) are characterized by the lack of the C2′, C3′ bond of the RNA ribose ring, creating an acyclic derivative of RNA. Single, but not multiple, UNA modification improves the stability and efficiency of gene silencing. Moreover, UNA modification in the seed region of guide strand limits miRNA-like off-target effects without compromising siRNA activity.1

Many other modifications with nonnucleotide compounds, such as N,N-diethyl-4-(4-nitronaphthalen-1-ylazo)-phenylamine (“ZEN”), have been reported to increase binding affinity and block degradation of anti-miRs when positioned at or near each end of that ASO.

In some aspects, the invention provides methods comprising delivering one or more polynucleotides, such as or one or more vectors as described herein (e.g., a vector encoding a decoy oligonucleotide discussed below), one or more transcripts thereof, and/or one or proteins transcribed therefrom, to a host cell. In some aspects, the invention further provides cells produced by such methods, and organisms (such as animals, plants, or fungi) comprising or produced from such cells. Conventional viral and non-viral based gene transfer methods can be used to introduce nucleic acids in mammalian cells or target tissues. Such methods can be used to administer nucleic acids encoding components of a BC 200 RNA decoy system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids are discussed above and include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4.186,183, 4,217.344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line.

In one aspect, the invention provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may be re-introduced into the human or non-human animal.

### Methods of Treating SLE

Encompassed herein are methods of treating lupus, particularly neuropsychiatric lupus, in a subject, comprising administering an effective amount of an antisense oligonucleotide. By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The terms “decrease”, “reduced”, “reduction”, or “inhibit” are all used herein to mean a decrease by a statistically significant amount. This can be a complete inhibition or activity or expression, or a partial inhibition. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

The term “lupus” describes an inflammatory disease caused when the immune system attacks its own tissues.

The term “systemic lupus erythematosus” or “SLE,” as used herein, refers to a chronic disease that causes inflammation in connective tissues, such as cartilage and the lining of blood vessels, which provide strength and flexibility to structures throughout the body. The signs and symptoms of SLE vary among affected individuals, and can involve many organs and systems, including the skin, joints, kidneys, lungs, central nervous system, and blood-forming (hematopoietic) system. SLE is one of a large group of conditions called autoimmune disorders that occur when the immune system attacks the body's own tissues and organs.

The term “NPSLE” or “neuropsychiatric lupus” as used herein refers to a type of SLE that has neuropsychiatric manifestations. In NPSLE, a CNS with a compromised blood brain barrier is targeted by autoimmune antibodies. An NPSLE phenotype may include increased levels of one or more autoantibodies which are immunologically specific for BC-RNA in a subject, which indicates that the subject is at greater risk for NPSLE, e.g., as compared to a control subject (i.e., one that does not have NPSLE or elevated levels of autoantibodies to BC-RNA). It is noted that comparisons to a positive control may also be used to determine a NPSLE phenotype, e.g., comparing the autoantibody levels in a subject to a control subject with NPSLE.

The clinical symptoms of NPSLE may include seizures, focal motor or sensory deficits, generalized disturbances, psychosis, organic brain syndrome, paresthesia without objective findings, clumsiness without objective findings, persistent headache, pseudopapilledema, benign intracranial hypertension, reactive depression, mood swings, cognitive disorders, severe anxiety, behavioral problems and other cognitive disfunction. Several studies have shown that the presence of NPSLE, independent of its etiology, is associated with greater morbidity and mortality. In early presentations of SLE there may not be any symptoms at all. However, as the disease progresses, symptoms, such as those discussed in this paragraph, manifest. A diagnosis of SLE is established on the basis of well characterized clinical signs and symptoms that are well known to skilled persons.

Since neuropsychiatric manifestations of SLE (NPSLE) are difficult to diagnose due to the diversity of clinical presentations, which include seizures, psychosis, cognitive dysfunction, and more, it is difficult to estimate the frequency of NPSLE. Cognitive impairment manifested as memory deficit is one of the most commonly observed symptoms in NPSLE patients, but is still poorly understood. It may be caused by a variety of mechanisms, both antibody and non-antibody mediated. Hypertension and accelerated atherosclerosis can also lead to cognitive impairment and confound the assessment of diseases-specific mechanisms. In animals, behaviors including, without limitation, repetitive, excessive self-grooming and failure to disengage from memorized but situation-conflicting information are indicative of cognitive dysfunction.

Cognitive flexibility is an essential underpinning of adaptive decision-making, and a number of brain regions, in particular prefrontal cortical regions, have been implicated in its maintenance. For instance, damage to the orbitofrontal cortex (OFC) has been reported to result in impaired conflict learning performance (Eichenbaum et al. 1983; Ragozzino 2007; Schoenbaum et al. 2009; Gruber et al. 2010; Tait et al. 2014). The OFC signals outcome expectancies on the basis of previous experience, and interactions between the OFC and other brain regions, such as the ventral tegmental area (VTA), serve to negotiate resolution of conflicts between such expectancies and actual outcomes (Schoenbaum et al. 2009).

Cognitive flexibility has been associated with prefrontal cortical areas, in particular the OFC, and the ASST has previously been used to assess prefrontal cortical function and cognitive flexibility. Thus, RNA control of neuronal translation is a mechanistic underpinning of higher brain function that, if impaired, can result in cognitive deficits. Thus, the present invention provides an approach to inhibit the cognitive impairment associated with NPSLE.

Some studies have also associated cognitive impairments in NPSLE with the presence of certain antibodies. These antibodies or cytokines contribute to cognitive problems in NPSLE.

One of ordinary skill knows that certain medications and procedures can slow the progression of the disease, and improve or manage the quality of life of the subject. These include nonsteroidal anti-inflammatory drugs, anticoagulation, and immuno-suppressive agents. In certain embodiments, these medications and procedures may be administered in combination with the decoy RNAs described herein to treat various forms of lupus and ameliorate symptoms thereof.

### Modes of Administration of Nucleic Acids Reducing Fibrosis Formation in SLE Subjects in Need Thereof.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

For intravenous administration, the compositions are packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates.

The formulation can contain a preservative to prevent the growth of microorganisms. Suitable preservatives include those that will not interfere with the activity of any of the active components of the formulation.

The formulation is typically buffered to a pH of 3-8 for parenteral administration upon reconstitution. Suitable buffers include, but are not limited to, phosphate buffers, acetate buffers, and citrate buffers.

The parenteral formulations described herein can be formulated for controlled release including immediate release, delayed release, extended release, pulsatile release, and combinations thereof.

In certain embodiments the uORF targeting nucleic acids of the invention can be incorporated into microparticles, nanoparticles, or combinations thereof that provide controlled release of the compounds and/or one or more additional active agents. In forms wherein the formulations contains two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the anti-lupus agent(s) described herein can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

Encapsulation or incorporation of decoys into carrier materials to produce decoy-containing microparticles can be achieved through known pharmaceutical formulation techniques.

To produce a coating layer of cross-linked protein (e.g., antibodies, ligands of receptors of interest) surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

### Therapeutic Interventions

The compositions and methods provided herein may be used to offer treatment options to the subgroup of lupus patients who suffer from anti-BC RNA neuropsychiatric autoimmunity. These compositions and methods may be especially beneficial to lupus patients who have a history of seizures and generate high-reactivity anti-BC abs (as shown in Table 1). As binding of such antibodies to BC200 RNA is essentially irreversible, we can, after establishing their titers in the circulation, apply a concentration of BC200 decoy that is calculated to quantitatively intercept circulating anti-BC abs.

### Kits and Articles of Manufacture

Any of the aforementioned products can be incorporated into a kit for the treatment of autoimmune diseases. In certain embodiments, the kit will allow clinicians to interfere with the actions of autoimmune antibodies that target BC200 RNA.

The following methods are provided in detail to facilitate the practice of the present invention.

### Materials and Methods for Example 1

Sera and antibodies. We collected sera from 69 patients diagnosed with SLE. These patients were categorized into three groups according to the criteria of the American College of Rheumatology for SLE (Tan et al., 1982) and for NPSLE (American College of Rheumatology, 1999). Sera were collected as follows (Table 1): Group I, SLE with seizure activity; Group II, SLE with organic brain syndrome (OBS); and Group III, SLE without diagnosed seizure or OBS involvement. Herein, SLE autoantibodies are identified by a patient code in which a number may be preceded by the letters S (indicating an SLE patient with a history of seizures) or OBS (indicating an SLE patient with organic brain syndrome). SLE patients with no history of seizures or OBS are identified by numbers without preceding letters.

TABLE 1
Autoimmune anti-BC RNA reactivity

| | | Anti-BC RNA reactivity | | | |
|---|---|---|---|---|---|
| Subjects | Diagnosis | + + | + | − | n |
| Group I | SLE with seizures | 10 (36%) | 12 | 6 | 28 |
| Group II | SLE with OBS | 4 (18%) | 13 | 5 | 22 |
| Group III | SLE | 4 (21%) | 11 | 4 | 19 |
| Group IV | Healthy subjects | 0 | 0 | 17 | 17 |
| Group V | Rheumatoid arthritis | 0 | 0 | 8 | 8 |
| Group VI | Ulcerative colitis | 0 | 0 | 1 | 1 |
| Group VII | Multiple sclerosis | 0 | 0 | 12 | 12 |

Reactivity is categorized as follows: + +, strong reactivity: +, weak reactivity −, no reactivity.
Reactivity to BC RNAs is specific to SLE patients (Groups I-III).
Samples from healthy subjects with no evidence of disease (healthy subjects, Group IV) and samples from patients with autoimmune diseases other than SLE (i.e. RA, UC, MS; Groups V-VII) were found to be nonreactive.
n refers to the total number of subjects in each group.
Anti-BC RNA reactivity was established by EMSA analysis using ³²2P-labeled BC200 RNA.

In addition, sera from non-SLE human subjects were collected as follows: Group IV, healthy with no evidence of disease (healthy subjects, HS); Group V, rheumatoid arthritis (RA); Group VI, ulcerative colitis (UC); and Group VII, multiple sclerosis (MS). SLE and UC sera were obtained from patients at the Division of Rheumatology of SUNY Down-state Medical Center (DMC). Samples were collected during periods of active disease when patients reported to the clinic. Clinical staff participating in sample collection were not cognizant of subsequent experimental sample usage (i.e., they were “blinded”). Samples were deidentified and end-point experimenters were not cognizant of sample types at the time of experimentation. Serum samples from HS, patients with RA, and patients with MS were obtained from Valley Biomedical and Proteogenex.

IgG was purified from human subject sera in a two-step protocol. In a first step, using a Nab Spin Kit (Thermo Fisher Scientific), serum samples were incubated in binding buffer on a Protein A/G spin column for 10 min. The column was washed with 400 μl of Nab Spin binding buffer at least three times. Antibodies were eluted from the column, using 400 μl of Nab Spin elution buffer, in three consecutive steps (elution 1-3). The three elution fractions were analyzed by SDS PAGE. In a second step, we used a NAb Protein G Spin Kit (Thermo Fisher Scientific) to purify IgG by removing any residual IgM. Elution 1 from step 1 was incubated in binding buffer on a Protein G spin column for 10 min. The column was washed with 400 μl of Nab Spin binding buffer three times. IgG was eluted from the column using 400 μl of Nab Spin elution buffer. IgG samples were dialyzed overnight at 4° C. against PBS in Slide-A-Lyzer dialysis cassettes (Thermo Fisher Scientific). Samples were analyzed by SDS PAGE. Samples from step 2 were used for electrophoretic mobility shift assay (EMSA) analysis and RNA transport analysis. Samples from step 1 (elution 1) were also used for EMSA analysis.

DNA synthesis and labeling. BC200 DNA was synthesized by PCR from plasmid pBC200 (Kondrashov et al., 2005) using primers as follows: BC200 forward primer, GGC CGG GCG CGG TGG CTCA (SEQ ID NO: 9); BC200 reverse primer, GGG GGG TTG TTG CTT TGA GGG (SEQ ID NO: 10). Synthesis and labeling was done using the Platinum PCR SuperMix (Thermo Fisher Scientific) according to the manufacturer's instructions. Then, 200 nM primers, 500 ng of plasmid pBC200 and 1 μl of dCTP (3000 ci/mmol, PerkinElmer) were added to 45 μl of master mix. PCR amplification conditions were as follows: 1 cycle of 1.5 min at 94° C., 45 s at 56° C., 1 min at 72° C., followed by 39 cycles of 30 s at 94° C., 45 s at 57° C., 1 min at 72° C., and a final cycle of 1 min at 94° C., 45 s at 57° C., 15 min 72° C. PCR products were run on a 2% agarose gel and purified from the gel using QIAquick gel extraction kits (Qiagen).

RNA expression constructs. Transcripts were generated from the following BC RNA plasmid constructs: (1) pBCX607 to generate full-length rat BC1 RNA (Muslimov et al., 2006, 2011), (2) pUC57_BC200 to generate full-length human BC200 RNA (Muslimov et al., 2018), (3) pBC_{IIL-A}: WC to generate BC1 RNA with the GA apical internal loop motif replaced with standard Wilson-Crick (WC) base pairs (Muslimov et al., 2006), (4) pBC1_{ΔU22} to generate BC1 RNA lacking U22 (Muslimov et al., 2006), (5) pBC1_{IL-B:WC} to generate BC1 RNA with the basal internal loop replaced with a standard WC base pair by a C61A transversion (Muslimov et al., 2006), (6) pBC200 to generate the 5′ domain of BC200 RNA (Muddashetty et al., 2002), and (7) pVL450-1 to generate the 3′ domain of BC200 RNA (Tiedge et al., 1993). U4 and U6 RNAs were generated from plasmids pSP6-U4 and pSP6-U6, respectively (Tiedge et al., 1991; Muslimov et al., 1997; Wang et al., 2005). All constructs were verified by sequencing.

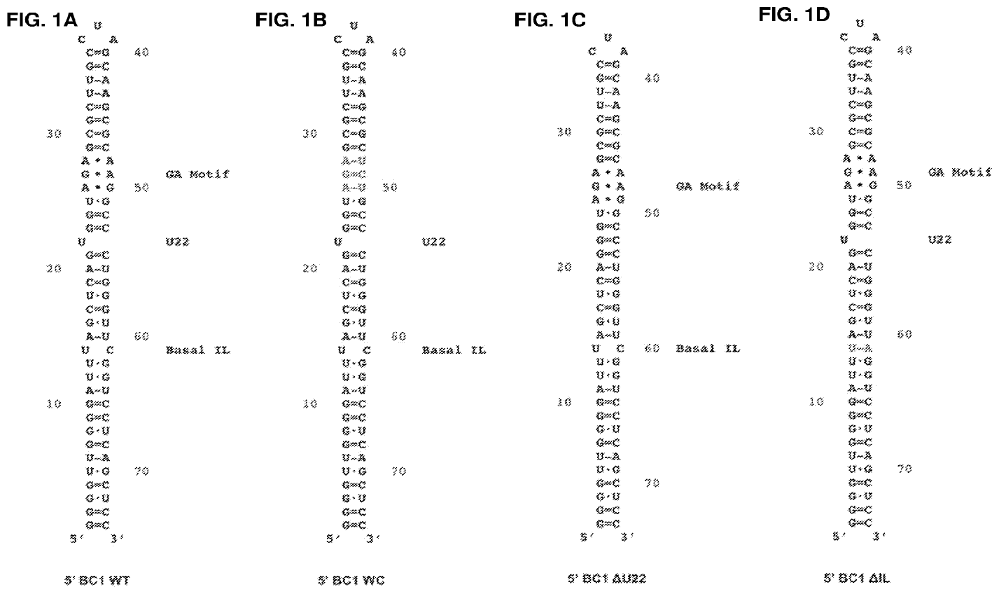

Plasmids were linearized, and ³⁵S- or ³²P-labeled transcripts were generated using T3, T7 RNA, or SP6 polymerase as described previously (Muslimov et al., 2004, 2006; Wang et al., 2005; and references cited in the preceding paragraph). Excess unlabeled UTP was added to reactions to ensure that labeled transcripts were full-length, and size, integrity, and stability of all transcripts were monitored as described previously (Muslimov et al., 1997, 2006, 2011). The secondary structures of WT and mutant 5 BC1 domains that were used in this work are shown in FIG. 1 (see also Muslimov et al., 2006).

Electrophoretic mobility shift assay (EMSA). RNAs were ³²P-labeled at 50,000 cpm per reaction (˜10 ng). RNAs were preheated at 70° C. for 10 min, allowed to cool for 5 min at room temperature, and incubated with antibodies using 1 μl of serum or purified IgG preparation (elution 1; see above, antibody purification) in a total of 20 μl of binding buffer (100 mM KCl, 3 mM MgCl₂, 2 mM DTT, 5% glycerol, and 20 mM HEPES, pH 7.6) for 30 min at 37° C. Heparin was added at 5 mg/ml and incubated for 10 min at room temperature to minimize unspecific RNA-protein interactions. When desired, unbound RNA was digested using RNase T1 at 30° C. for 10 min. RNA-antibody complexes were resolved by 5% PAGE (TBE precast gels, Bio-Rad) in Tris/borate/EDTA (TBE) running buffer (prerun for 1 h at 30 mA, run for 2 h at 25 mA) and visualized by autoradiography.

EMSA competition experiments (competition between IgGs and hn-RNP A2) were performed as follows (Muslimov et al., 2018). Recombinant full-length hnRNP A2 was expressed (Muslimov et al., 2011, 2014, 2018) from plasmid pET-9c (Munro et al., 1999). Purified hnRNP A2 (10 nM) was preincubated with radiolabeled BC1 or BC200 RNA, or derivatives, in 10 μl of binding buffer at 37° C. for 30 min. IgG was added (1:1 molar ratio IgG to hnRNP A2), and incubation was continued for another 30 min, or longer as detailed in the figures. RNA-antibody complexes were resolved on 8% polyacrylamide gels (ratio acrylamide/bisacrylamide 19:1) in 90 mM Tris-borate, pH 8.3, 15 mM MgCl₂, at room temperature for 12 h at 15 V (Muslimov et al., 2018).

EMSA results were quantified as follows. Dried gels were exposed to autoradiographic film; films were scanned, and bands representing immune complexes were analyzed using ImageJ (NIH). Signal intensities were recorded in relative signal intensity units (SIUs). Signal intensities of <500 SIUs were considered background level (i.e., absence of signal) and classified as no reactivity. Signal intensities of 500-20,000 SIU were classified as low reactivity. Signal intensities of >20,000 SIU were classified as high reactivity.

Cell culture. Sympathetic neurons were maintained in low-density primary cultures (Muslimov et al., 2006, 2011, 2014). Neurons were prepared from superior cervical ganglia isolated from E19-E20 Sprague Dawley rat embryos (of either sex) and grown on glass coverslips pre-coated with 100 μg/ml filter-sterilized poly-D-lysine (Sigma-Aldrich). Composition of culture media was as follows: 50% (v/v) of Ham's F12 and DMEM (Life Technologies), BSA (500 μg/ml; Calbiochem), bovine insulin (10 g/ml; Sigma-Aldrich), rat transferrin (20 μg/ml; Jackson ImmunoResearch Laboratories), L-glutamine (20 μg/ml; Life Technologies), sodium selenite (5 ng/ml; Sigma-Aldrich), and nerve growth factor (β-NGF, 100 ng/ml; Harlan Bioproducts for Science). Dendritic growth was induced by addition of basement membrane extract (Matrigel, 100 g/ml; Collaborative Biomedical Products) on the third day in vitro. Cytosine arabinofuranoside (Ara-C, 2 μM; Sigma-Aldrich) was added on the second and fifth days after plating to minimize proliferation of non-neuronal cells. Work with vertebrate animals was approved by the SUNY DMC Institutional Animal Care and Use Committee.

Cultured sympathetic neurons were microinjected with RNA (Muslimov et al., 2004, 2006, 2011, 2014) as follows. RNAs were ³⁵S-radiolabeled at 3×10⁶ cpm/μl. Perikaryal injection was performed at volumes of a few femtoliters per pulse. Lucifer yellow (LY, 0.4%) was coinjected to monitor the injection process and to ensure that the experimental manipulations did not cause alterations in cell morphology, including dendritic extent and arborization, over the course of the experiments (Muslimov et al., 2006, 2014). Transcript stability was ascertained preinjection by PAGE, postinjection by measuring average integrated total signal intensities per injected cell (Muslimov et al., 2006, 2011, 2014), and by incubation with brain extract (Muslimov et al., 2011, 2014).

We have opted for microinjection of radiolabeled transcripts to introduce RNAs into cells. The reason for this preference is twofold (Muslimov et al., 2014). In our experience, microinjection affords exquisite control of amounts of RNA introduced. Therefore, in combination with the high sensitivity of radiolabel detection, microinjection allows us to maintain the number of introduced RNA molecules at levels lower than those of respective endogenous RNA molecules (Muslimov et al., 2006, 2014). We remain committed to using radiolabeled RNAs because architectural GA motifs, central to dendritic targeting of BC RNAs, are quite intolerant of nucleotide substitutions and of introduced side chains (such as fluorophores) as they may disrupt motif structure and, as a result, interfere with targeting (Goody et al., 2004; Muslimov et al., 2011, 2014).

For co-injection experiments, radiolabeled BC1 or BC200 RNAs were incubated with purified IgG preparations for 30 min in PBS at 37° C. before microinjection. In bath application experiments, purified IgG preparations were added to culture media at a dilution of 1:100 and incubated for various periods of time before microinjection of BC1 RNA or BC200 RNA. IgG bath incubation time ranged from 1 h to overnight. The full effect of bath-applied IgGs (e.g., diminished dendritic targeting after application of IgG SLE S1 or S6) was observed after incubation periods of 8 h or more. In the experiments presented in the Results section, 8 h incubation periods were chosen for bath-applied IgGs.

Injection of mice with sera and antibodies. We adhered to the protocol of the Diamond laboratory (Kowal et al., 2006) for this approach. Male BALB/c mice (age 6-8 weeks; Jackson Laboratory) were injected intravenously with SLE or non-SLE sera or purified IgG fractions. The retroorbital route (Schoch et al., 2014) was used for intravenous injections. Sera were injected on day 1 (100 μl, right side) and on day 2 (100 μl, left side). Intravenous injections of purified IgG fractions (0.5 mg/ml in 100 μl of saline) were performed analogously. 15 min after each serum or IgG injection, lipopolysaccharide (LPS, 3 mg/kg) was injected intraperitoneally. LPS intraperitoneal injections were repeated 2 d after the last intravenous injection. Animals were killed 2 d later. We anticipated that this time frame would be sufficiently long to allow for clearance of preexisting endogenous BC1 RNA from dendrites (Muslimov et al., 2011) but not long enough to risk clearance of injected antibodies from circulation and brain (Schoch et al., 2014).

Animals were intravenously injected with sera or purified IgG fractions as follows: IgG SLE S1, 8 animals; serum SLE S1, 2 animals; serum SLE S6, 2 animals; IgG HS4, 4 animals; serum HS4, 4 animals; IgG RA1, 1 animal; IgG MS1, 1 animal. BC1 RNA localization data from IgG SLE S1, serum SLE S1, and serum SLE S6 injected animals did not qualitatively or quantitatively differ from each other and were therefore combined for analysis (total number of animals: 12). Analogously, BC1 RNA localization data from IgG HS4, serum HS4, IgG RA1, and IgG MS1 injected animals did not qualitatively or quantitatively differ from each other and were therefore combined for analysis (total number of animals: 10).

In situ hybridization. We performed in situ hybridization to examine BC1 RNA dendritic localization deficits in vivo. Animals that had been injected with SLE anti-BC sera or abs, and animals that had been injected with non-SLE sera or abs, were perfusion fixed, their brains prepared, cryosectioned at 10 μm thickness (coronal sections were generated), and hybridized with anti-BC1 RNA probes (or “sense”-strand control probes) as described previously (Tiedge et al., 1991; Tiedge, 1991; Lin et al., 2001). We continue to prefer ³⁵S-labeled probes (Tiedge et al., 1991; Lin et al., 2001; Muslimov et al., 2018) as we find that non-radioactively labeled BC1 RNA probes are prone to generating unspecific labeling artifacts. After extensive high-stringency washes, sections were dried and exposed to autoradiographic film and subsequently to autoradiographic emulsion (Lin et al., 2001). Sections were counterstained with cresyl violet (Lin et al., 2001).

Microscopy. Photomicrographs [dark field, phase contrast, and differential interference contrast (DIC Nomarski)] were taken on a Nikon Microphot-FXA microscope with a Digital Sight DS-Fil 5-megapixel charge-coupled device (CCD) camera (Eom et al., 2014; Muslimov et al., 2018). The following objectives were used: (1) Plan Fluor 10X/0.30, 160/0.17; (2) Ph2 Plan 20X/0.50, DL 160/0.17; (3) Ph3 DL Plan 40X/0.65, 160/0.17; Plan 20/0.70 DIC 160/0.17. Illustrations were generated using Photoshop and final figures were arranged in Illustrator (both Adobe Systems).

Experimental design and statistical analysis. For dendritic transport experiments using sympathetic neurons in primary culture, the experimental design followed a previously published protocol (Muslimov et al., 1997, 2004, 2006, 2011, 2014). Each RNA was injected at a range of concentrations (covering at least one order of magnitude) to ensure that observed dendritic targeting patterns were independent of amounts injected. The number of neurons and dendrites analyzed is given for each experiment. Dendritic localization of injected RNAs was quantified as follows. Silver grain densities were measured along dendritic extents at 50 μm interval points, up to a length of 250 μm. A relative signal of 100% was assigned to levels at the base of dendrites (0 μm points). Statistical significance was examined using one-way ANOVA followed by Dunnett's multiple-comparisons analysis in which dendritic RNA levels were compared at all interval points (Muslimov et al., 1997, 2004, 2006, 2011, 2014). Herein, data points and error bars are given as mean ±SEM.

For in vivo BC1 RNA localization experiments, ³⁵S-labeled RNA probes were used as described above (Lin et al., 2001). Quantification and statistical analysis were performed as follows. Silver grain densities were obtained as a measure of relative labeling signal (Muslimov et al., 2004, 2018). Signal intensities were established from hybridized cryosections of hippocampal CA1 in stratum pyramidale (center), in stratum oriens at a distance of 50 μm from the edge of stratum pyramidale, and in stratum radiatum at distances of 50, 100, and 150 μm from the edge of stratum pyramidale, Statistical significance was examined using one-way ANOVA followed by Tukey's or Dunnett's multiple-comparisons analysis, comparing signal intensities in strata pyramidale, oriens, and radiatum at sample points defined above (Muslimov et al., 2018).

Nonparametric Spearman's rank-order correlation analysis was per-formed as described previously (Mus et al., 2007). The Spearman's approach was used to examine a correlation of SLE with or without neuropsychiatric manifestations (seizures, OBS) on one hand with the strength of anti-BC autoimmune reactivity on the other. Results were expressed in the form of a Spearman's rank correlation coefficient (Spearman's ρ or rₛ). The Mann-Whitney U test was used to compare the distribution of anti-BC autoimmune reactivity in SLE versus non-SLE samples.

Quantitative and statistical analysis was performed using Prism (GraphPad) and SPSS Statistics (IBM, RRID:SCR_002865) software.

### Example 1

### Identification of Lupus Subset for Treatment Using BC RNAs

Although clinical manifestations of neuropsychiatric lupus are well recognized, the underlying molecular-cellular alterations have been difficult to determine. We report that sera of a subset of lupus patients contain autoantibodies directed at regulatory brain cytoplasmic (BC) RNAs. These antibodies, which we call anti-BC abs, target the BC RNA 5′ domain noncanonical motif structures that specify dendritic delivery. Lupus anti-BC abs effectively compete with RNA transport factor heterogeneous nuclear ribonucleoprotein A2 (hnRNP A2) for access to BC RNAs. As a result, hnRNP A2 is displaced, and BC RNAs are impaired in their ability to reach synapto-dendritic sites of function. The results reveal an unexpected link between BC RNA autoantibody recognition and dendritic RNA targeting. Cellular RNA dysregulation may thus be a contributing factor in the pathogenesis of neuro-psychiatric lupus.

### Results

### Screening of Sera

We screened sera from a total of 107 subjects, including 69 SLE patients, for autoantibodies directed against human BC200 RNA. Subjects were categorized as belonging to one of seven groups as listed in Table 1. Subjects had been diagnosed as follows: SLE with seizures (Group I), SLE with organic brain syndrome (Group II), SLE (Group III), HS (Group IV), RA (Group V), UC (Group VI), and MS (Group VII).

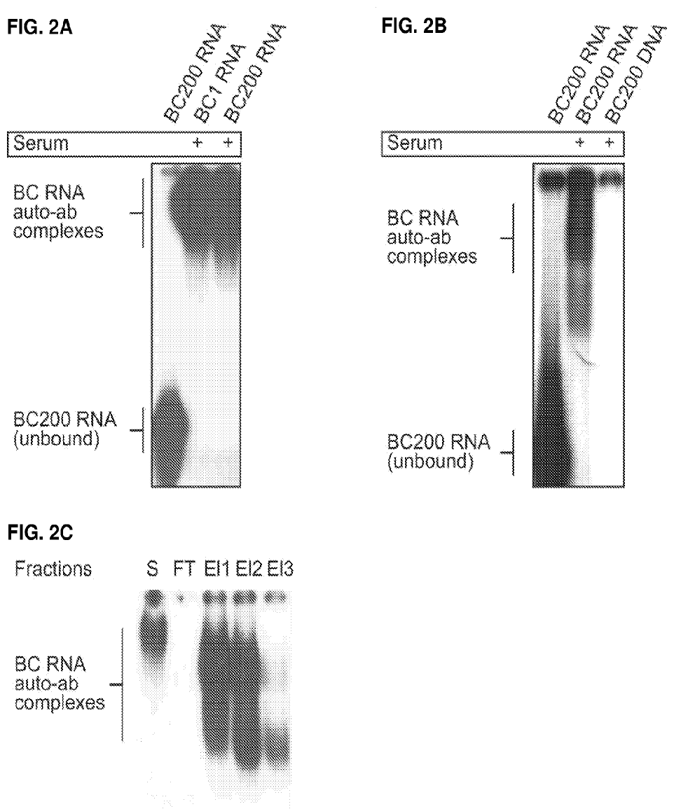

Our screen revealed that of 69 SLE patient sera (Groups I-III), 54 tested positive for antibodies against BC RNAs, 18 of them with high reactivity (Table 1). Electrophoretic mobility shift assay (EMSA) analysis indicated that positive SLE sera were reactive to human BC200 RNA and rat BC1 RNA as they formed lower-mobility immune complexes with both RNAs (FIG. 2A).

Select SLE autoantibodies have been reported to bind double-stranded DNA (dsDNA) (Uccellini et al., 2012; Mader et al., 2017). We tested whether SLE anti-BC sera would also recognize genomic BC200 DNA. Results in FIG. 2B show that BC200 dsDNA, in contrast to BC200 RNA, was not mobility-shifted. The data indicate that SLE anti-BC abs reactivity is specifically RNA-directed.

We subsequently affinity-purified antibodies from the 18 high-reactivity SLE sera (using protein A/G and protein G Sepharose columns; see Materials and Methods) and determined that, in each case, anti-BC reactivity was associated with the IgG class of immunoglobulins (FIG. 2C). These antibodies will be referred to as anti-BC IgGs.

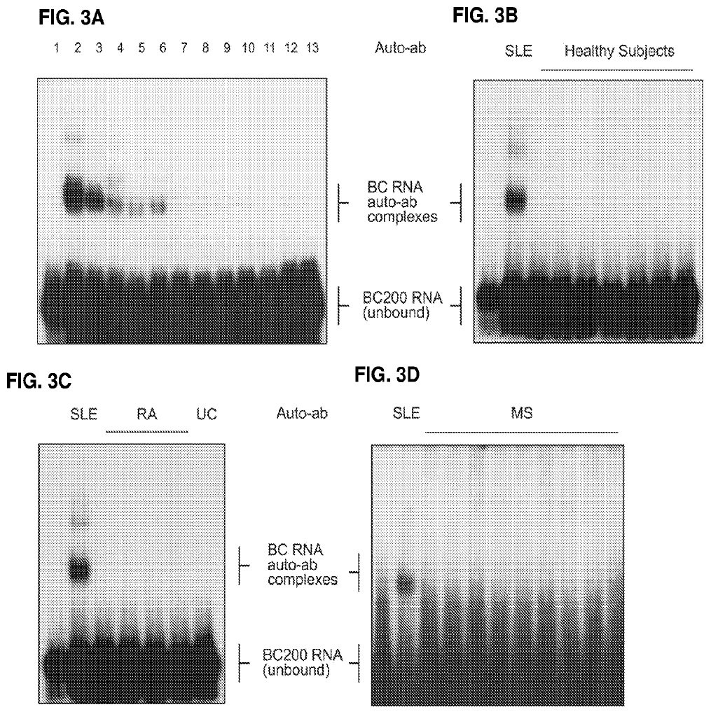

Among the SLE IgG samples tested, reactivity to BC200 RNA ranged from strong (FIG. 3A, lanes 2 and 3) to weak (FIG. 3A, lanes 4-6) to undetectable (FIG. 3A, lanes 7-13). Strong anti-BC RNA immune reactivity was detected in 36% of SLE cases with a history of seizures (Table 1, Group I), in 18% of SLE cases with organic brain syndrome (OBS; Table 1, Group II), and in 21% of cases without neurological symptoms (Table 1, Group III). Nonparametric Spearman's rank-order correlation analysis revealed a strong correlation between the strength of SLE anti-BC autoimmune reactivity and the occurrence of neuropsychiatric manifestations (Spearman's correlation coefficient r^{s}=0.89, 95% confidence interval 0.83-0.93, p<0.0001, n=69).

By contrast, anti-BC RNA immune reactivity was not detected with IgGs from HS without evidence of disease (FIG. 3B; Table 1, Group IV) or with IgGs from patients with autoimmune diseases other than SLE (Table 1, Groups V-VII). Specifically, no anti-BC RNA immune reactivity was found with IgGs from patients with RA (FIG. 3C), UC (FIG. 3C), or MS (FIG. 3D). We conclude that the observed anti-BC RNA autoimmune responses are specific for SLE and are not triggered in patients with other systemic (RA, MS) or localized (UC) autoimmune diseases, or in HS (Mann-Whitney U test, comparison of anti-BC autoimmune reactivity levels between SLE and non-SLE samples, p<0.0001, n=69).

### Molecular-Structural Targets of SLE Anti-BC Abs

BC RNA functionality is physically compartmentalized along structural domains and resident architectural motifs (lacoangeli and Tiedge, 2013; Eom et al., 2018). To be able to appreciate the functional implications of SLE anti-BC RNA autoimmune reactivity, it was therefore important to identify the molecular epitope target(s) of anti-BC abs. Rodent BC1 RNA and primate BC200 RNA are organized into tripartite structures in which a 5′ stem-loop domain is linked to a 3′ stem-loop domain by a single-stranded A-rich central domain (DeChiara and Brosius, 1987; Tiedge et al., 1993; Skryabin et al., 1998; Rozhdestvensky et al., 2001; lacoangeli and Tiedge, 2013). While the BC1 and BC200 3′ stem-loop structures mediate translational control (Lin et al., 2008; Eom et al., 2011, 2014, 2018), the 5′ stem-loops carry spatial codes that, acting as DTEs, specify RNA delivery to synapto-dendritic sites of function (Muslimov et al., 1997, 2006, 2011).

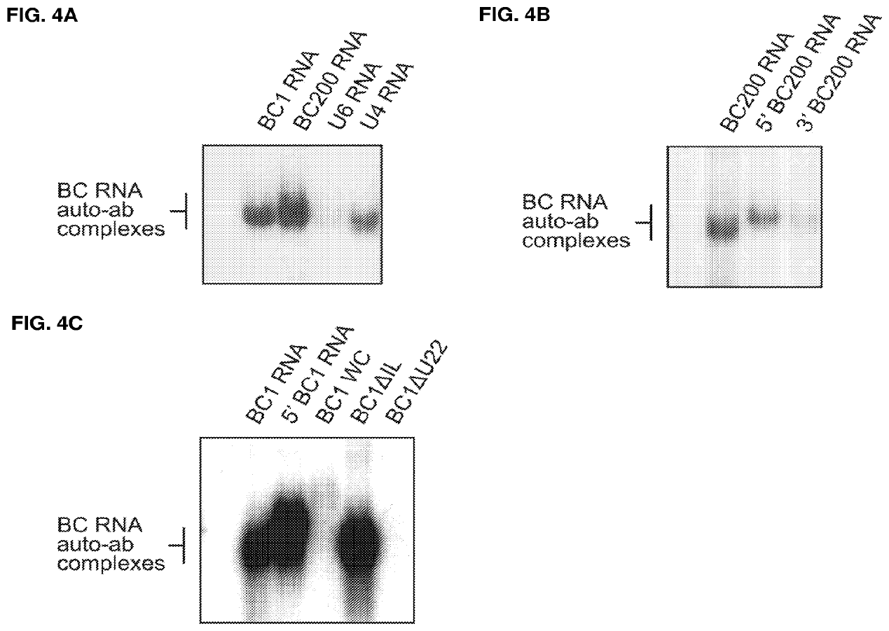

We performed EMSA analysis with purified SLE IgG preparations to examine interactions with human BC200 RNA, rat BC1 RNA, and respective subdomains or mutants. We found that SLE anti-BC RNA reactivity (using IgG SLE S6, i.e., purified IgG from an SLE patient with a history of seizures #6) was directed at both human BC200 RNA and rat BC1 RNA (FIG. 4A), confirming the results obtained with sera (FIG. 2). Anti-BC reactivity was stronger toward human BC200 RNA than to rat BC1 RNA (FIG. 4A). Limited cross-reactivity of IgG SLE S6 was observed with small nuclear (sn) U4 RNA but not with U6 snRNA (FIG. 4A). U4 and U6 RNAs are spliceosome components; it is noted that U4 RNA (Vidovic et al., 2000; Klein et al., 2001), but not U6 RNA (Karaduman et al., 2006), contains a noncanonical GA motif which, although different from BC RNA GA motifs described below, may be underlying the observed cross-reactivity. For IgG SLE S6, cross-reactivity with U4 RNA was estimated at <5% of the reactivity level with BC200 RNA (FIG. 4A). Finally, we found that SLE IgG anti-BC reactivity was directed at the 5′ stem-loop domain as the 3′ domain was poorly recognized (FIG. 4B).

In double-stranded RNA domains, it is frequently architectural motifs rather than sequence content that serve as recognition codes for RNA binding proteins (Noller, 2005; Leontis et al., 2006). The 5′ domains of BC1 RNA and BC200 RNA contain several such motifs (Skryabin et al., 1998; Muslimov et al., 2006, 2011, 2018; lacoangeli and Tiedge, 2013; Eom et al., 2018) which for BC1 RNA include an apical noncanonical GA motif, an unpaired U-residue at position 22 (U22), and a basal internal loop (FIG. 1). The BC1 GA motif features noncanonical (i.e., non-WC) purine·purine interactions in which tandem G·A/A·G pairs engage in Hoogsteen-type hydrogen bonding (Muslimov et al., 2006, 2011; lacoangeli and Tiedge, 2013; Eom et al., 2018) (FIG. 1).

We investigated whether any of the three BC1 RNA 5′ architectural motifs are recognized as antigenic epitopes by SLE anti-BC abs. SLE anti-BC IgGs were used with WT BC1 RNA and with BC1 RNA derivatives in which one of these motifs had been altered by point mutations (see FIG. 1 for structures of the mutants used). We found that in BC1 RNA, the 5′ domain noncanonical GA motif and unpaired U22 were both indispensable for SLE autoantibody recognition (FIG. 4C). The 5′ domain basal internal loop, in contrast, was recognition irrelevant (FIG. 4C).

A requirement of the noncanonical GA motif and unpaired U22 for SLE anti-BC ab recognition is remarkable as it is precisely these structural attributes in the 5′ BC1 domain that have previously been identified as instrumental in specifying dendritic targeting competence (Muslimov et al., 2006, 2011; Eom et al., 2018). Thus, the noncanonical GA motif and unpaired U22 are resident and requisite structural components of the 5′ BC1 DTE, and both motifs (but not the basal internal loop) are indispensable for dendritic delivery (Muslimov et al., 2006, 2011). The above data thus indicate that SLE anti-BC abs interact with exactly those 5′ BC1 DTE architectural attributes that are responsible for dendritic targeting.

Molecular Competition of SLE Anti-BC Abs with RNA Transport Factor hnRNP A2 for Access to BC RNAs

The results presented in the preceding section indicate that SLE anti-BC abs can target RNA structural epitopes that serve as DTE spatial codes for BC RNA synapto-dendritic delivery. Subcellular transport of BC1 and BC200 RNAs is mediated by the RNA transport factor hnRNP A2 via interactions with 5′ DTEs (Muslimov et al., 2006, 2011; Iacoangeli and Tiedge, 2013; Eom et al., 2018). The question is thus raised whether SLE anti-BC abs compete with hnRNP A2 for binding to BC1 and BC200 RNAs.

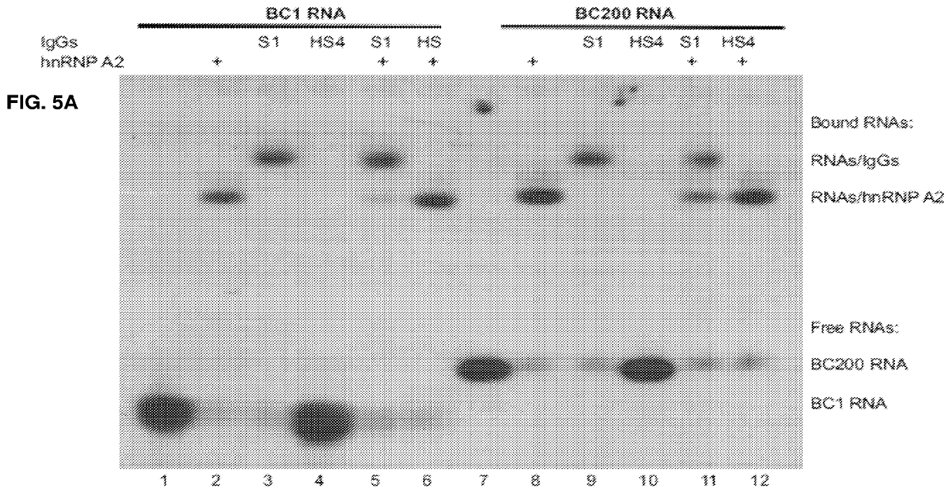

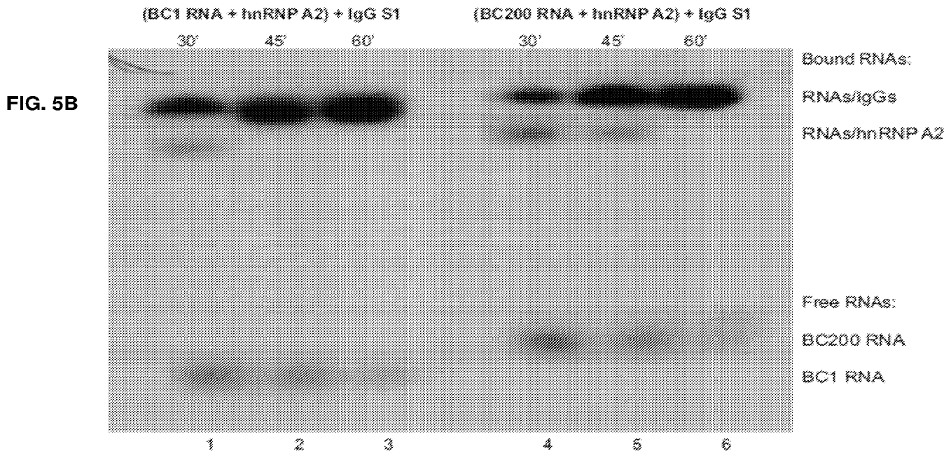

To address this question, we performed EMSA analysis using purified IgGs from two SLE patient sera (IgG SLE S1 and IgG SLE S6). We first confirmed (FIG. 5A) that binding of IgG SLE S1 to both BC1 RNA and BC200 RNA was robust, whereas no binding was observed with IgGs from an HS (IgG HS4). Equally robust was binding of recombinant hnRNP A2 to BC1 RNA and BC200 RNA (FIG. 5A). Next, in an EMSA competition analysis, BC1 or BC200 RNA was preincubated with hnRNP A2. After 30 min, IgG SLE S1 was added to the reaction mix, and incubation was continued for another 30 min. At this time, hnRNP A2 was almost completely displaced from BC1 RNA and partially displaced from BC200 RNA (FIG. 5A). When incubation was extended to a total of 60 min, hnRNP A2 was completely displaced from both RNAs (FIG. 5B). By contrast, no displacement from either RNA was observed with IgG HS4 (FIG. 5A).

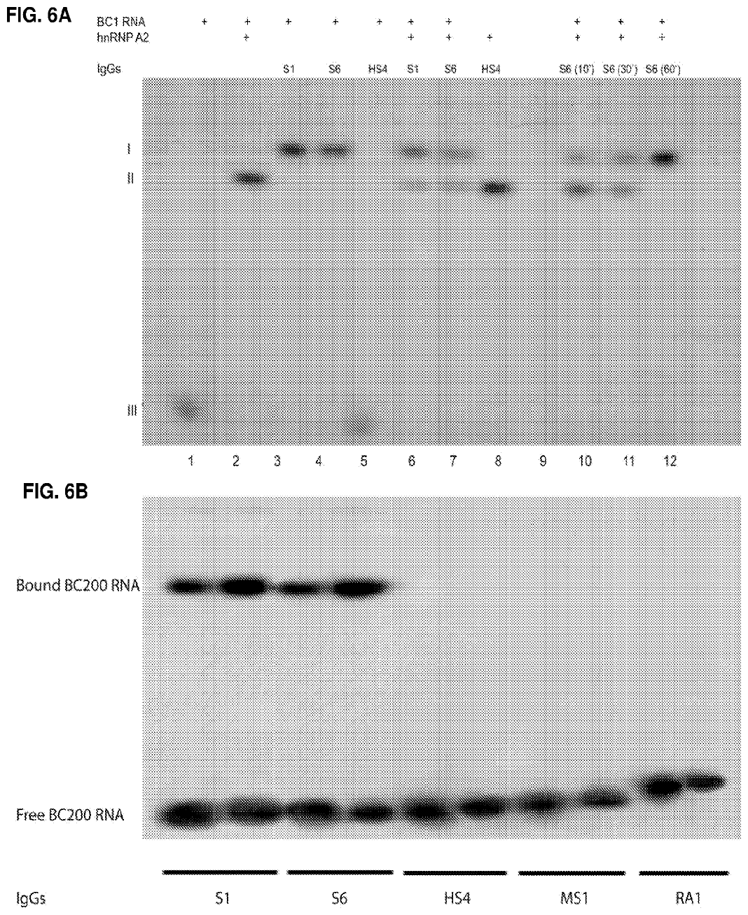

BC RNA-anti-BC ab complex formation, and displacement of hnRNP A2, was analogously observed when IgG SLE S6 was used instead of IgG SLE S1 in EMSA competition analysis (FIG. 6A). Failure to form such complexes, in contrast, was apparent with IgG MS1 (MS patient) and with IgG RA1 (RA patient), in addition to IgG HS4 (FIG. 6B). The combined data indicate that binding of SLE anti-BC abs specifically and effectively prevents BC1 RNA and BC200 RNA from interacting with hnRNP A2.

Together, the evidence presented herein indicates that SLE anti-BC RNA autoimmune reactivity is primarily directed against relevant structural motifs in the 5′ DTE domains, and that interaction of SLE anti-BC abs with such motifs displaces RNA transport factor hnRNP A2 from BC RNAs. These results suggested to us that competition will affect BC RNA dendritic delivery and may be useful to interfere with this pathological process.

### SLE Anti-BC Abs: BC RNA Dendritic Transport Impairments

We hypothesized that molecular displacement of hnRNP A2 by SLE anti-BC abs detrimentally impacts dendritic delivery of BC RNAs. We applied RNA transport assays using microinjection methodology with sympathetic neurons in primary culture (Muslimov et al., 1997, 2004, 2006, 2011, 2014) to address this question. We microinjected perikaryal regions of cultured neurons with ³⁵S-radiolabeled full-length BC1 RNA or BC200 RNA. Progression of labeled RNAs into and along dendrites was monitored over various periods of time up to a total of 4 h. In agreement with earlier data, BC1 RNA and BC200 RNA were transported to reach distal dendritic tips within the 4 h period (Muslimov et al., 1997, 2006, 2011).

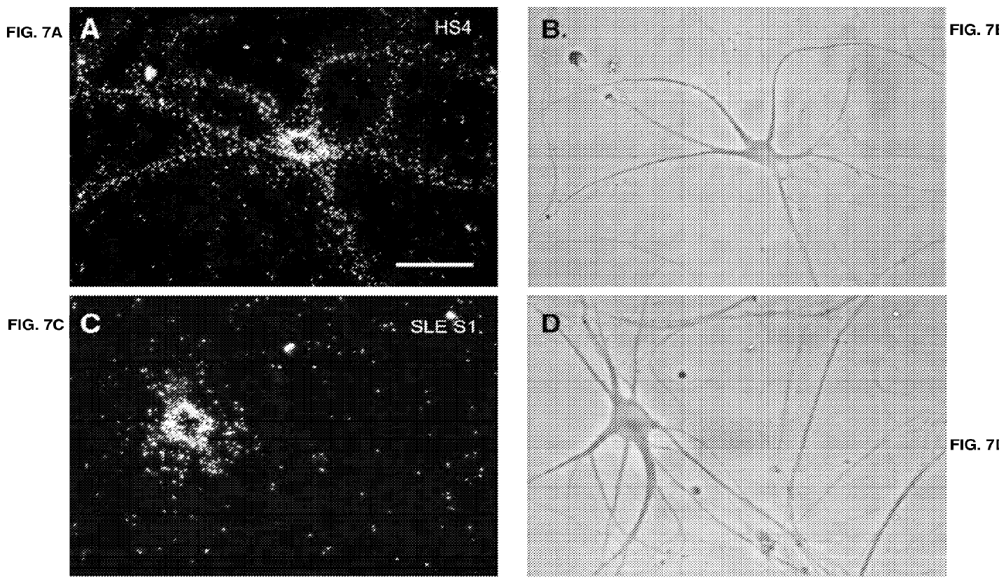

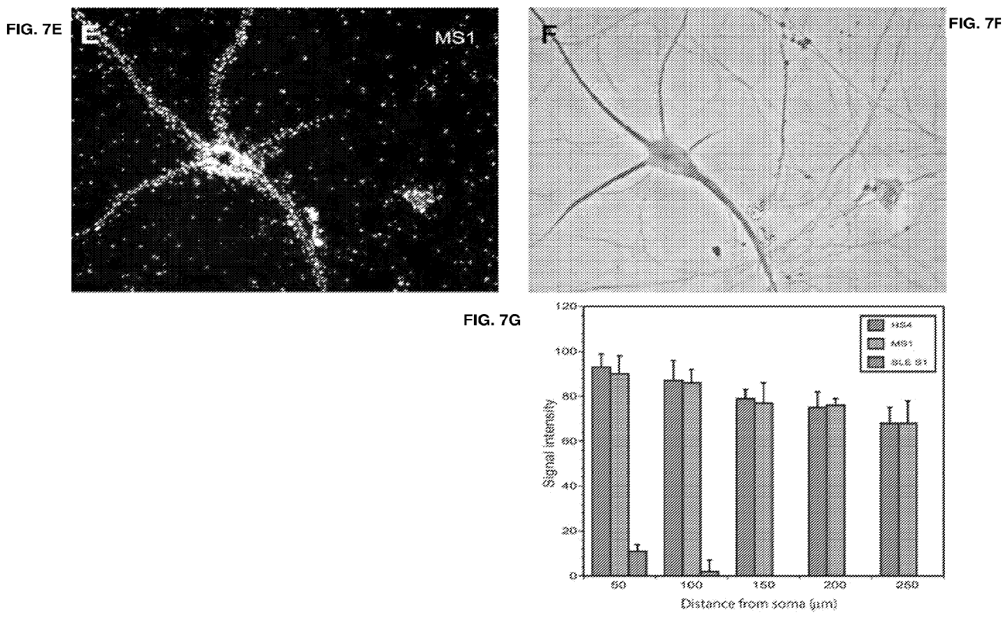

In the next step, we preincubated radiolabeled BC1 RNA with purified IgGs from an HS (IgG HS4) for 30 min before coinjection into sympathetic neurons in primary culture. The resulting labeling signal extended along the entire dendritic extent (FIGS. 7A, 7B), indicating that IgG HS4 did not impact dendritic delivery of rodent BC1 RNA. In clear contrast, BC1 RNA that had been preincubated and coinjected with IgG SLE S1 showed a somatically restricted distribution profile (FIG. 7C, 7D). The data indicate that SLE anti-BC abs were interfering with the transport of BC1 RNA along dendritic arborizations. By contrast again, when the same experiment was performed using IgG MS1 (i.e., autoimmune IgG from a MS patient), BC1 RNA was delivered normally to dendrites (FIG. 7E, 7F), and the somato-dendritic distribution pattern was not significantly different from that observed in the presence of coinjected IgG from an HS (FIG. 7G). For quantitative analysis (FIG. 7G), signal intensities were established along dendritic extents at 50 μm interval points, up to a length of 250 μm. Statistical analysis was performed using one-way ANOVA followed by Dunnett's multiple-comparisons analysis [comparison of dendritic BC1 RNA levels after coinjection with IgG HS4 (FIG. 7A, 7B) with levels after coinjection with IgG SLE S1 (FIG. 7C, 7D), p<0.0001 for all interval points; comparison with levels after coinjection with IgG MS1 (FIG. 7E, 7F), p>0.95 for all interval points]. The data, together with those of FIG. 5, support the hypothesis that SLE autoimmune abs (but not MS autoimmune abs), by displacing transport factor hnRNP A2, abolish or significantly reduce dendritic targeting of BC1 RNA.

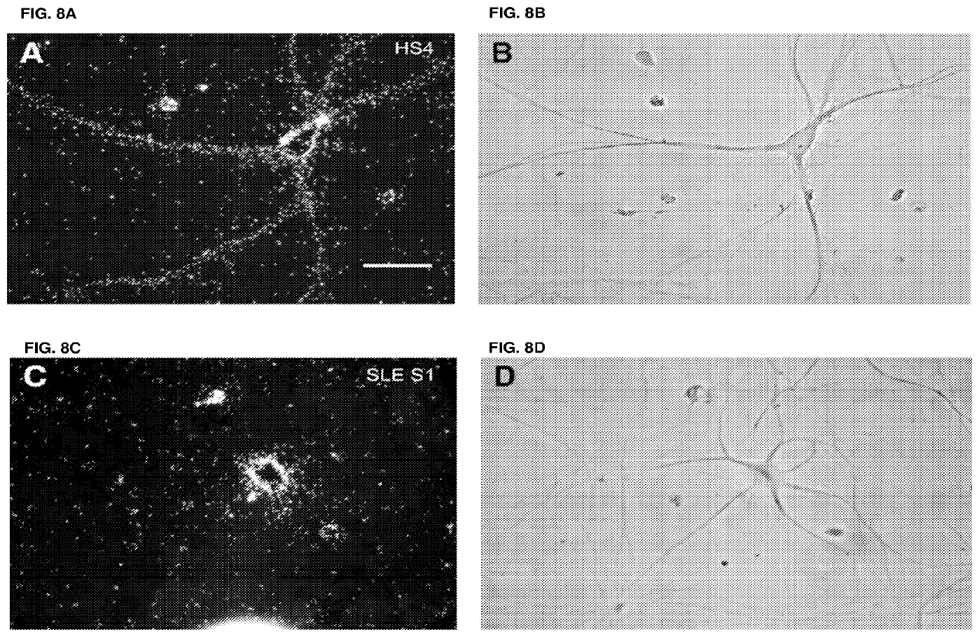

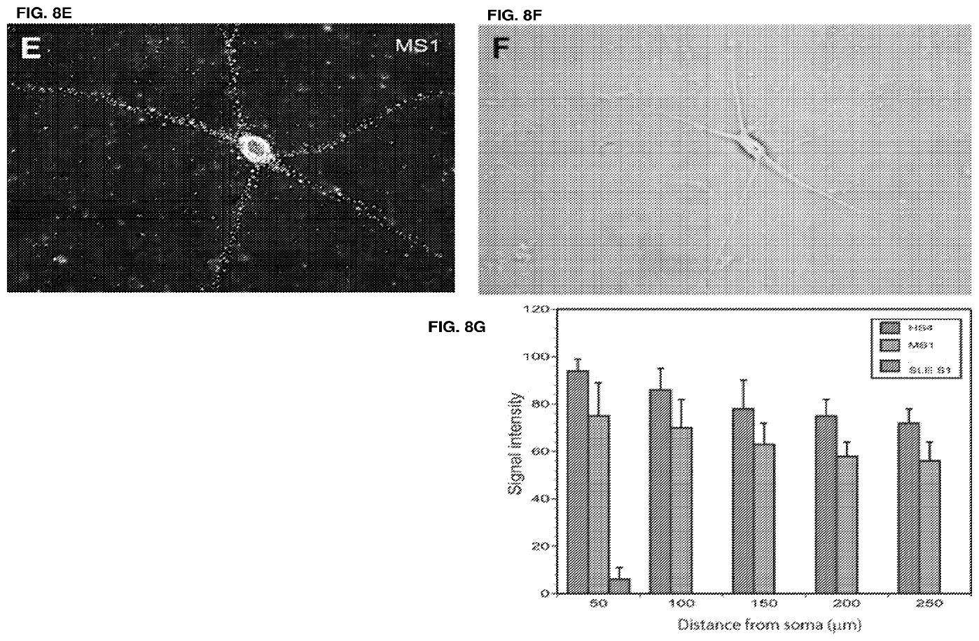

Further experiments were performed to test the above hypothesis. As detailed herein, rodent BC1 RNA and primate BC200 RNA are not phylogenetic orthologs but appear to operate in neurons in a functionally-mechanistically analogous manner (lacoangeli and Tiedge, 2013; Eom et al., 2018). To determine whether this functional analogy extends to dendritic targeting being susceptible to SLE anti-BC autoimmune reactivity, we performed RNA transport experiments with rodent BC1 RNA and primate BC200 RNA in parallel. As is shown in FIG. 8, dendritic delivery of human BC200 RNA was significantly diminished after preincubation and coinjection of radiolabeled RNA with IgG SLE S1 (FIG. 8C, 8D). By contrast, preincubation and coinjection with IgG HS4 (FIG. 8A, 8B) or IgG MS1 (FIG. 8E, 8F) did not result in reduced dendritic delivery of BC200 RNA (FIG. 8G). Quantitative analysis (FIG. 8G) was performed using one-way ANOVA followed by Dunnett's test [comparison of dendritic BC200 RNA levels after coinjection with IgG HS4 (FIG. 8A, 8B) with levels after coinjection with IgG SLE S1 (FIG. 8C, 8D), p<0.0001 for all interval points; comparison with levels after coinjection with IgG MS1 (FIG. 8E. 8F), p>0.62 for all interval points]. We conclude that dendritic targeting of rodent BC1 RNA (FIG. 7) and primate BC200 RNA (FIG. 8) is analogously inhibited by SLE IgGs but not by HS or MS patient IgGs.

Before being able to engage cytoplasmic regulatory BC RNAs, anti-BC abs will have to enter cells. Neurons have been shown to internalize IgGs, with uptake occurring via clathrin-dependent Fcγ receptor-mediated endocytosis (Elkon and Casali, 2008; Congdon et al., 2013; Douglas et al., 2013; Kazim et al., 2017; Rhodes and Isenberg, 2017). We reasoned that preincubation of cultured neurons with bath-applied SLE anti-BC IgGs would, after antibody uptake, cause BC RNA transport impairments identical or similar to those observed after antibody coinjection.

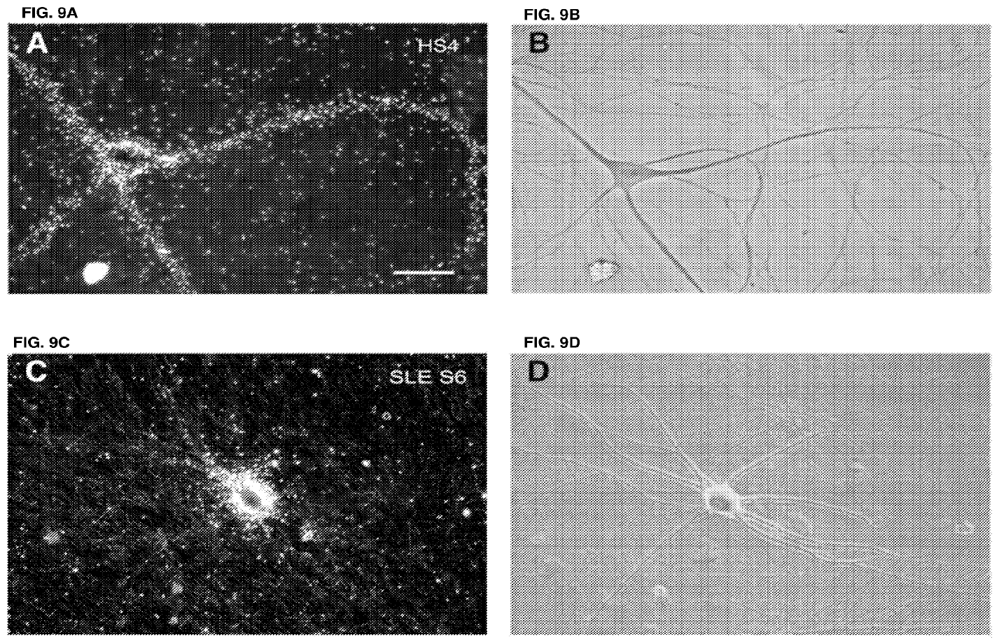

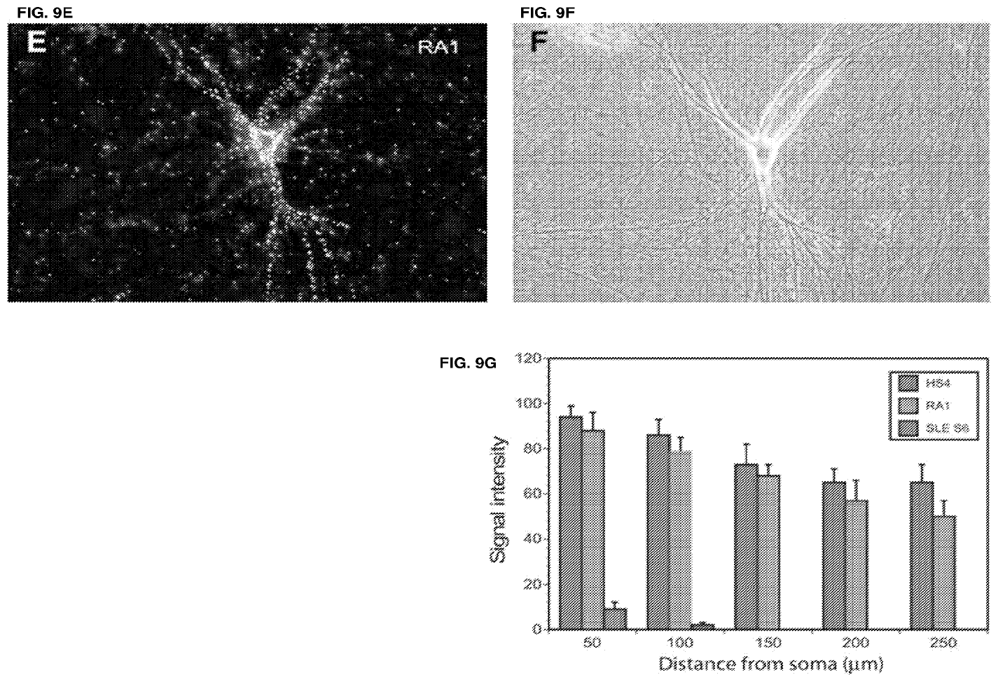

Two sets of experiments were performed to test this prediction. In the first set, we bath-applied IgGs to sympathetic neurons in culture for various periods of time (see Materials and Methods) before perikaryal microinjection of radiolabeled rat BC1 RNA. Data presented in FIG. 9 show that an 8 h bath application of IgG SLE S6 (FIG. 9C, 9D), but not of IgG HS4 (FIG. 9A, 9B) or IgG RA1 (FIG. 9E, 9F), resulted in significantly reduced dendritic targeting of BC1 RNA (FIG. 9G). Quantitative analysis (FIG. 9G) was performed using one-way ANOVA followed by Dunnett's test [comparison of dendritic BC1 RNA levels after bath application of IgG HS4 (FIG. 9A, 9B) with levels after bath application of IgG SLE S6 (FIG. 9C, 9D), p<0.0001 for all interval points; comparison with levels after bath application of IgG RA1 (FIG. 9E, 9F), p>0.73 for all interval points].

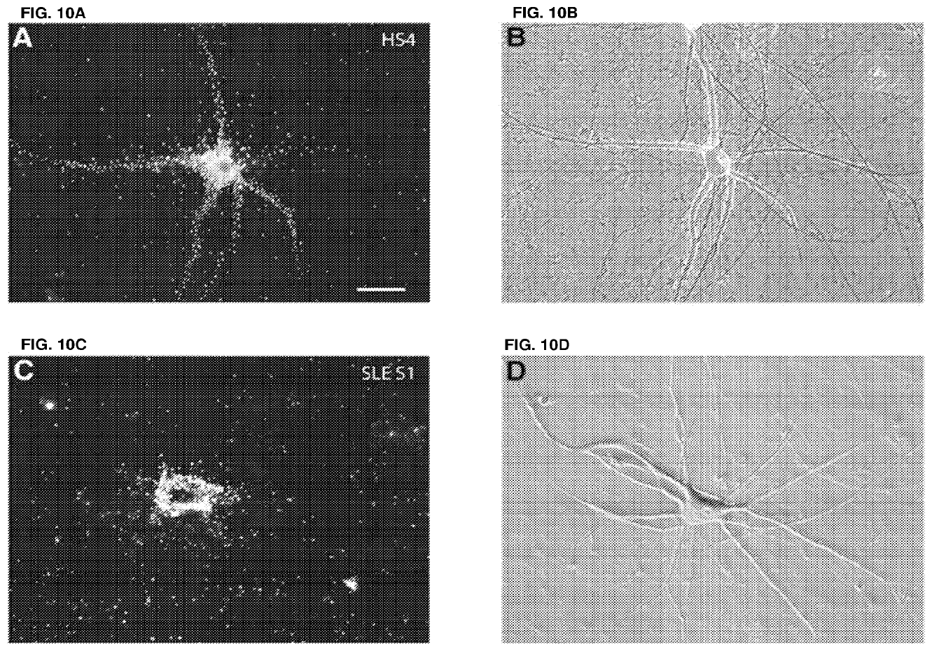

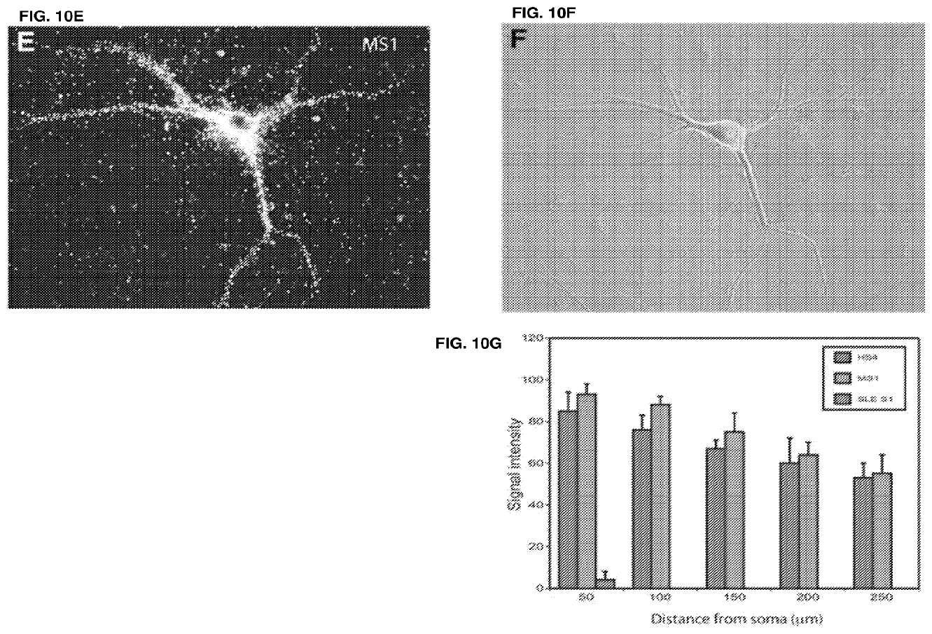

In a second set of experiments, we used radiolabeled human BC200 RNA for microinjection of cultured neurons after bath application of IgGs (FIG. 10). Results obtained in these experiments were analogous to those obtained with rat BC1 RNA. BC200 RNA was delivered along the entire dendritic extent after bath application of IgG HS4 (FIG. 10A, 10B) or IgG MS1 (FIG. 10E, 10F). By contrast, dendritic targeting was significantly reduced after bath application of IgG SLE S1 (FIG. 10C, 10D). Quantitative analysis (FIG. 10G) was performed using one-way ANOVA followed by Dunnett's test [comparison of dendritic BC200 RNA levels after bath application of IgG HS4 (FIG. 10A, 10B) with levels after bath application of IgG SLE S1 (FIG. 10C, 10D), p<0.0001 for all interval points; comparison with levels after bath application of IgG MS1 (FIG. 10E, 10F), p>0.73 for all interval points].

We conclude from the above results that bath-applied IgGs are taken up by cultured neurons, and that anti-BC IgGs from SLE patients thus internalized are able to interfere with the dendritic delivery of rat BC1 RNA and human BC200 RNA. By contrast, delivery of BC1 RNA or BC200 RNA to synapto-dendritic domains appears unaffected when IgGs from non-SLE subjects (including HS as well as MS patients and RA patients) are used in coinjection or bath-application approaches. The combined data indicate that SLE anti-BC IgGs, recognizing 5′ dendritic targeting elements and competing with hnRNP A2, specifically disrupt delivery of regulatory BC RNAs to dendrites.

### SLE Anti-BC Abs: Mislocalization of BC1 RNA In Vivo

The above results indicate that SLE anti-BC abs are internalized by neurons in primary culture, after which they interfere with BC RNA dendritic localization. We next asked whether in vivo, circulating antibodies can gain access to the brain and are internalized by neurons in sufficient concentrations to impact dendritic localization of endogenous BC1 RNA. This issue was addressed by intravenous injections of mice in which purified SLE anti-BC IgGs or purified other, non-SLE IgGs were injected intravenously as described previously (Kowal et al., 2006; see also Materials and Methods). Each intravenous injection of IgG was directly followed by an intraperitoneal injection of LPS (Kowal et al., 2006). LPS was applied to breach the BBB and thus to allow injected, circulating IgGs access to the brain (Kowal et al., 2004, 2006; Mader et al., 2017). Another round of LPS intraperitoneal injections was performed 48 h later. Animals were killed after 2 more days and their brains processed for in situ hybridization directed at endogenous BC1 RNA.

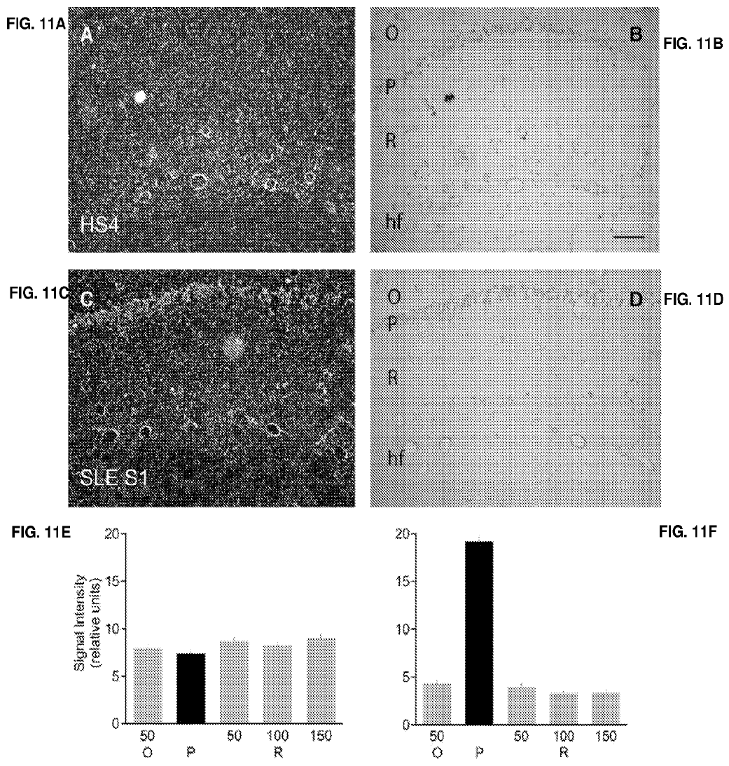

FIG. 11 shows that in hippocampal area CA1, the distribution of BC1 RNA is normal after intravenous injection of IgG HS4, i.e., purified IgG from an HS. Thus, in this case, robust BC1 RNA signal intensities are detectable in synapto-dendritic strata oriens and radiatum, with signal intensities similar to those in somatic stratum pyramidale (FIG. 11A, 11B). This result reflects an enrichment of BC1 RNA in dendritic arborizations of CA1 pyramidal cells, as has previously been reported for WT rats (Lin et al., 2001) and mice (Muslimov et al., 2018). In striking contrast, in CA1 of mice injected with IgG SLE S1, the distribution of the BC1 RNA labeling signal was significantly increased in somatic stratum pyramidale and significantly decreased in synapto-dendritic strata oriens and radiatum (FIG. 11C, 11D). These results indicate (1) that injected antibodies were able to cross the BBB and enter neurons, and (2) that purified IgG from an SLE patient with neuropsychiatric manifestations, but not IgG from an HS, prevented BC1 RNA from significantly localizing to CA1 pyramidal cell dendrites, thus causing its accumulation in neuronal cell bodies.

The above qualitative observations were further confirmed by quantitative analysis of BC1 RNA labeling intensities in strata oriens, pyramidale, and radiatum obtained from animals injected with SLE anti-BC abs (n=12; see Materials and Methods) and those obtained from animals injected with non-SLE abs (n=10) (FIG. 11E, 11F). One-way ANOVA was followed by Tukey's multiple-comparisons test in which signal intensities in stratum pyramidale (center), in stratum oriens at a distance of 50 μm from edge of stratum pyramidale, and in stratum radiatum at distances of 50, 100, and 150 μm from edge of stratum pyramidale, were compared between animals injected with SLE anti-BC abs and those injected with non-SLE abs: p<0.0001 for all sample points.

In CA1 of animals injected with non-SLE abs (FIG. 11E), signal intensities in stratum pyramidale tended to be lower than those in neighboring strata oriens and radiatum (one-way ANOVA, p=0.043). Post hoc analysis using Dunnett's multiple-comparisons test revealed that signal intensities in stratum pyramidale were significantly lower than those in stratum radiatum at 150 μm (p=0.018) but not significantly lower than those in stratum radiatum at 50 μm (p=0.067), those in stratum radiatum at 100 μm (p=0.349), or those in stratum oriens at 50 μm (p=0.71). By contrast, in CA1 of animals injected with SLE anti-BC abs (FIG. 11F), signal intensities in stratum pyramidale were significantly higher than those in neighboring strata oriens and radiatum (one-way ANOVA followed by Dunnett's multiple-comparisons test of strata pyramidale versus oriens and radiatum at above distances: p<0.0001 for all sample points).

In conclusion the above in vivo data indicate that after entering the brain (by crossing the LPS-breached BBB) and being taken up by neurons, SLE anti-BC abs can target BC1 RNA and interfere with its localization to synapto-dendritic domains.

### Discussion

BC RNAs constitute a small group of scRNAs of which rodent BC1 RNA and primate BC200 RNA have been most extensively investigated (for review, see lacoangeli and Tiedge, 2013; Eom et al., 2018). Neuronal BC1 and BC200 RNAs are translational regulators that operate in the activity-dependent control of local protein synthesis in synapto-dendritic domains (Eom et al., 2011, 2014, 2018). Translational control competence resides in BC1 and BC200 RNA 3′ stem-loop domains which contain identical C-loop motif structures (Eom et al., 2011). The BC RNA 3′ C-loop motifs feature noncanonical nucleotide interactions of the A·A trans-WC/Hoogsteen and of the C·A cis-WC/sugar edge subtypes. These C-loop motif noncanonical interactions are requisite for high-affinity binding to eIF4B and translational control competence (Eom et al., 2011, 2014; lacoangeli and Tiedge, 2013).

Extending this analogy, the BC1 and BC200 RNA 5′ stem-loop domains harbor similar GA motif structures (Skryabin et al., 1998; Muslimov et al., 2006, 2011; Iacoangeli and Tiedge, 2013; Eom et al., 2018). The noncanonical BC RNA GA motifs act as spatial codes to specify dendritic targeting (Muslimov et al., 2006, 2011; lacoangeli and Tiedge, 2013; Eom et al., 2018). The BC1 RNA 5′ GA motif features tandem G·A/A·G pairings, in the trans-Hoogsteen/sugar edge hydrogen bonding format, that are required for dendritic delivery (Lescoute et al., 2005; Muslimov et al., 2006). BC1 RNA U22 also contributes to dendritic targeting competence, whereas a basal internal loop does not (Muslimov et al., 2006). In addition, work with transgenic mouse lines expressing mutant BC1 RNAs has confirmed a 5′ DTE requirement for dendritic localization (Robeck et al., 2016). However, apparent methodological insufficiencies have been noted (Eom et al., 2018; see also technical comment published online with Robeck et al., 2016).

In the design of our experimental approach, we took into consideration the following information (see also introduction). First, because BC1 RNA and BC200 RNA are functional analogs rather than phylogenetic orthologs, it was important to establish whether interactions of the two RNAs with SLE anti-BC abs are similarly analogous. For this reason, molecular and cellular experiments were performed with BC1 RNA and BC200 RNA in parallel. Second, since BC1 and BC200 RNAs, because of their distinct phylogenetic background, feature unrelated primary structures but, as a result of retroposition-mediated functional convergence (Iacoangeli and Tiedge, 2013), identical or similar higher-order-structure architectural motifs, we surmised that any anti-BC ab recognizing both RNAs would recognize such motifs rather than nucleotide sequences

We found that SLE anti-BC RNA autoimmune antibodies do in fact recognized both BC RNAs as an SLE anti-BC ab recognizing BC200 RNA would also recognize BC1 RNA, albeit at lower reactivity. This result suggests that the BC1-BC200 functional-mechanistic analogy (lacoangeli and Tiedge, 2013; Eom et al., 2018) is also maintained with respect to autoantibody interactions. This functional correspondence extends even further as SLE anti-BC abs target mechanistically analogous double-stranded RNA motif structures. Reactivity of anti-BC abs was found directed at BC1 and BC200 RNA 5′ DTE stem-loop domains, structural elements that constitute the “transport centers” of BC RNAs as they carry spatial codes to specify dendritic delivery (Muslimov et al., 2006, 2011, 2018; lacoangeli and Tiedge, 2013; Eom et al., 2018).

SLE anti-BC abs thus engage the 5′ transport centers of BC RNAs (rather than their 3′ business centers, the C-loop motifs that mediate translational control). In the 5′ BC1 DTE, GA motif noncanonical purine·purine pairings, together with unpaired nucleotide U22, are required for SLE autoantibody recognition. Precisely the same structural attributes have previously been identified as requisite determinants of dendritic targeting competence (Muslimov et al., 2006, 2011). Transport factor hnRNP A2, essential for BC RNA dendritic targeting, specifically interacts with 5′ noncanonical GA motif structures (Muslimov et al., 2011). We now find that SLE anti-BC IgGs compete with hnRNP A2 for access to BC RNAs, effectively displacing the transport factor at a 1:1 molar ratio.

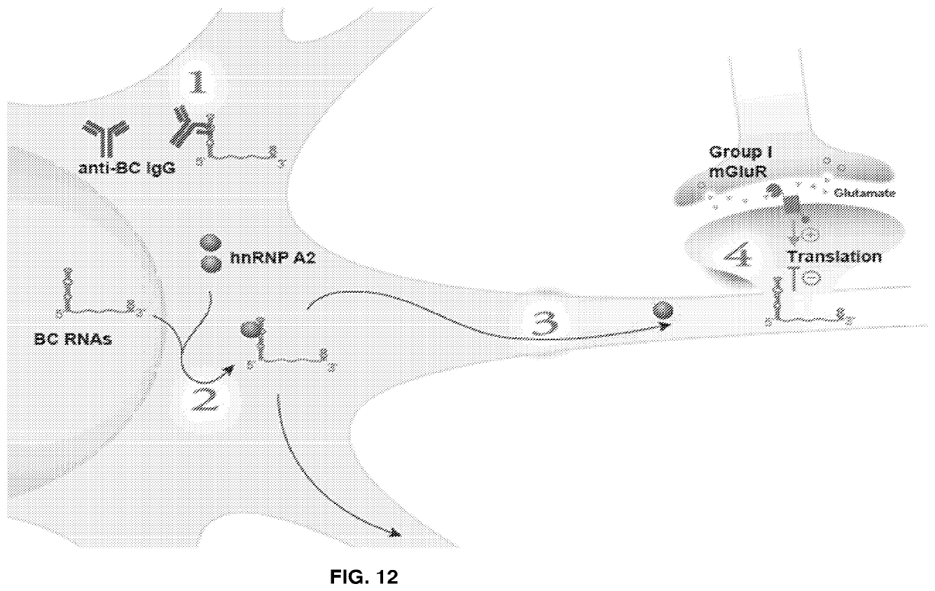

Molecular competition between SLE anti-BC abs and hnRNP A2 is of immediate functional relevance as it predicts BC RNA transport impairments as a consequence of the transport factor's diminished ability to access 5′ DTE structures. RNA transport assays corroborated this hypothesis: SLE anti-BC abs significantly decreased dendritic delivery of both BC1 and BC200 RNAs. Two types of experimental approaches were performed. (i) BC RNAs were preincubated with SLE anti-BC IgGs or other IgGs, before comicroinjection into cultured neurons. (ii) Cultured neurons were preincubated with SLE anti-BC IgGs or other IgGs, by bath application, before BC RNA microinjection. In either approach, application of SLE anti-BC IgGs resulted in significantly reduced dendritic delivery of BC1 and BC200 RNAs. The data are in agreement with accumulating evidence showing that neurons internalize IgGs via clathrin-dependent Fcγ receptor-mediated endocytosis, that such internalization is requisite for the clearance of intracellular antigens, and that internalized autoantibodies have the potential to disrupt intracellular functions (Elkon and Casali, 2008; Congdon et al., 2013; Douglas et al., 2013; Kazim et al., 2017; Rhodes and Isenberg, 2017). In the case of SLE anti-BC IgGs, intracellular molecular mechanisms are impacted as RNA transport factor hnRNP A2 is displaced from BC RNA DTEs, a displacement that causes RNA mislocalization as BC RNAs are now predominantly retained in neuronal somata rather than being delivered to synapto-dendritic domains (FIG. 12).

In in vivo experiments with mice, we applied LPS to permeabilize the BBB (Kowal et al., 2004, 2006) and injected autoimmune and other antibodies intravenously. SLE anti-BC abs, but not non-SLE abs, caused a significant change in the somato-dendritic distribution of endogenous BC1 RNA in hippocampal CA1: BC1 RNA, normally present at robust levels in dendritic domains, now collapsed back into a predominantly somatic localization. Because SLE anti-BC abs, but not non-SLE abs, target BC RNA DTEs and prevent transport factor hnRNP A2 from DTE binding, we suggest that intravenous injected abs reach the CNS where they are taken up by neurons, after which SLE anti-BC abs but not non-SLE abs prevent anterograde delivery of endogenous BC1 RNA to synapto-dendritic domains. Such failure to deliver, together with clearance of preexisting endogenous BC1 RNA from dendritic domains (by mechanisms that remain to be established), results in significantly diminished synapto-dendritic localization and substantial mislocalization of the RNA to somatic domains.

The absence, or severely reduced presence, of regulatory BC RNAs in synapto-dendritic domains is expected to give rise to phenotypic alterations that are similar to, but possibly milder than, those observed in the BC1 KO animal model. Global lack of BC1 RNA in such animals causes neuronal hyperexcitability that manifests in the form of prolonged epileptiform discharges in slice preparations and seizure susceptibility in vivo, as well as cognitive impairment (Zhong et al., 2009, 2010; Chung et al., 2017; lacoangeli et al., 2017). Identical or similar phenotypic alterations have been observed in the CGG-repeat animal model (Muslimov et al., 2018). In these animals, generated as a model for the fragile X premutation disorder (Bontekoe et al., 2001), the 5′ UTR of Fmr1 mRNA harbors expanded CGG triplet repeats in the range of 55-200 units (Oostra and Willemsen, 2009). Phenotypic hallmarks of the fragile X premutation disorder include epilepsy and cognitive impairment in young patients and neurodegeneration and motor dysfunction in advanced-age patients. (Hagerman et al., 2010, 2016; Hagerman, 2013). Our previous work (Muslimov et al., 2011, 2018) has shown that expanded CGG repeats, which form noncanonical stem-loop structures featuring purine·purine base pairs, sequester transport factor hnRNP A2 and cause significantly reduced synapto-dendritic delivery of BC1 RNA in vivo. Thus, epilepsy and cognitive impairment in young CGG-repeat animals are consequential to BC1 RNA dendritic transport deficits and subcellular mislocalization (Muslimov et al., 2018).

We conclude that BC RNA mislocalization can be caused by reduced functional availability of hnRNP A2 as a result of CGG-repeat competition (fragile X premutation) or of anti-BC autoantibody competition (SLE). Epilepsy and cognitive impairment are phenotypic consequences of either type of competition. Such overall correspondence notwithstanding, important differences are noted as SLE clinical presentations have been known to be notoriously diverse (Mader et al., 2017). Several underlying causes, not mutually exclusive, may contribute to this diversity. While auto-IgGs are taken up via Fcγ receptor-mediated endocytosis (Elkon and Casali, 2008; Congdon et al., 2013; Douglas et al., 2013; Kazim et al., 2017; Rhodes and Isenberg. 2017), the efficiency of this mechanism may vary among neuronal cell types. Autoantibodies have to traverse a compromised BBB for access to CNS neurons (Abbott et al., 2003; Mader et al., 2017). Depending on the nature and location of BBB disruption(s), SLE autoantibody-impacted CNS areas and resulting neurological symptoms may vary. Finally, low-level crossreactivity was observed with U4 snRNA, raising the question of whether phenotypic consequences may also result from engaging spliceosomal U4 RNA in addition to BC RNAs.

Why are SLE anti-BC abs directed at BC RNA 5′ transport centers but not at 3′ business centers? The data reveal that 5′ GA motifs, but not 3′ C-loop motifs, resemble viral RNA structures and may thus be mistaken for “nonself” intruders. The HIV Rev response element stem-loop IIb, for example, contains a noncanonical RNA motif with G·A and G·G base pairings (Jain and Belasco, 1996). The similarity to BC RNA GA motifs may “deceive” an immune system into treating BC RNAs as inherently nonself. Alternatively, a viral infection may trigger an antiviral RNA immune response with significant and potentially long-lasting crossreactivity against BC RNAs. Such molecular mimicry (Mader et al., 2017) may underlie anti-RNA autoimmune responses in general.

### REFERENCES

Abbott N J, Mendonc a LL, Dolman D E (2003) The blood-brain barrier in systemic lupus erythematosus. Lupus 12:908-915.
American College of Rheumatology (1999) The American College of Rheumatology nomenclature and case definitions for neuropsychiatric lupus syndromes. Arthritis Rheum 42:599-608.
Arbuckle M R, McClain M T, Rubertone M V, Scofield R H, Dennis G J, James J A, Harley J B (2003) Development of autoantibodies before the clinical onset of systemic lupus erythematosus. N Engl J Med 349:1526-1533.
Barrat F J, Elkon K B, Fitzgerald K A (2016) Importance of nucleic acid recognition in inflammation and autoimmunity. Annu Rev Med 67:323-336.
Bontekoe C J, Bakker C E, Nieuwenhuizen I M, van der Linde H, Lans H, de Lange D, Hirst M C, Oostra B A (2001) Instability of a (CGG)98 repeat in the Fmr1 promoter. Hum Mol Genet 10:1693-1699.
Briz V, Restivo L, Pasciuto E, Juczewski K, Mercaldo V, Lo A C, Baatsen P, Gounko N V, Borreca A, Girardi T, Luca R, Nys J, Poorthuis R B, Mansvelder H D, Fisone G, Ammassari-Teule M, Arckens L, Krieger P, Meredith R, Bagni C (2017) The non-coding RNA BC1 regulates experience-dependent structural plasticity and learning. Nat Commun 8:293.
Chu J L, Brot N, Weissbach H, Elkon K (1991) Lupus antiribosomal P anti-sera contain antibodies to a small fragment of 28S rRNA located in the proposed ribosomal GTPase center. J Exp Med 174:507-514.
Chung A, Dahan N, Alarcon J M, Fenton A A (2017) Effects of regulatory BC1 RNA deletion on synaptic plasticity, learning, and memory. Learn Mem 24:646-649.
Cimaz R, Guerrini R (2008) Epilepsy in lupus. Lupus 17:777-779.
Congdon E E, Gu J, Sait H B, Sigurdsson E M (2013) Antibody uptake into neurons occurs primarily via clathrin-dependent Fc7 receptor endocytosis and is a prerequisite for acute tau protein clearance. J Biol Chem 288:35452-35465.
Crow M K (2010) Long interspersed nuclear elements (LINE-1): potential triggers of systemic autoimmune disease. Autoimmunity 43:7-16.
DeChiara T M, Brosius J (1987) Neural BC1 RNA: cDNA clones reveal nonrepetitive sequence content. Proc Natl Acad Sci USA 84:2624-2628.
Douglas J N, Gardner L A, Levin M C (2013) Antibodies to an intracellular antigen penetrate neuronal cells and cause deleterious effects. J Clin Cell Immunol 4:1-7.
Elkon K, Casali P (2008) Nature and functions of autoantibodies. Nat Clin Pract Rheumatol 4:491-498.
Elkon K B, Bonfa E, Brot N (1992) Antiribosomal antibodies in systemic lupus erythematosus. Rheum Dis Clin North Am 18:377-390.
Eom T, Berardi V, Zhong J, Risuleo G, Tiedge H (2011) Dual nature of translational control by regulatory BC RNAs. Mol Cell Biol 31:4538-4549.
Eom T, Muslimov I A, Tsokas P, Berardi V, Zhong J, Sacktor T C, Tiedge H (2014) Neuronal BC RNAs cooperate with eIF4B to mediate activity-dependent translational control. J Cell Biol 207:237-252.
Eom T, Muslimov I A, Iacoangeli A, Tiedge H (2018) Dendritic targeting and regulatory RNA control of local neuronal translation. In: The Oxford handbook of neuronal protein synthesis (Sossin W, ed). New York: OUP.
Goody T A, Melcher S E, Norman D G, Lilley D M (2004) The kink-turn motif in RNA is dimorphic, and metal ion-dependent. RNA 10:254-264.
Grandin K (2010) The Nobel Prizes 2009. Stockholm: Nobel Foundation.
Hagerman P (2013) Fragile X-associated tremor/ataxia syndrome (FXTAS): pathology and mechanisms. Acta Neuropathol 126:1-19.
Hagerman R J, Wheeler A, Fitzpatrick S, Hunter J (2016) Premutation-associated disorders in childhood and adulthood. In: FXTAS, FXPOI, and other premutations disorders, Ed 2 (Tassone F, Hall D A, eds), pp 241-262. Switzerland: Springer.
Hagerman R, Hoem G, Hagerman P (2010) Fragile X and autism: intertwined at the molecular level leading to targeted treatments. Mol Autism 1:12.
Hoet R M, Pieffers M, Stassen M H, Raats J, de Wildt R, Pruijn G J, van den Hoogen F, van Venrooij W J (1999) The importance of the light chain for the epitope specificity of human anti-U1 small nuclear RNA autoantibod-ies present in systemic lupus erythematosus patients. J Immunol 163: 3304-3312.
Iacoangeli A, Tiedge H (2013) Translational control at the synapse: role of RNA regulators. Trends Biochem Sci 38:47-55.
Iacoangeli A, Dosunmu A, Eom T, Stefanov D G, Tiedge H (2017) Regulatory BC1 RNA in cognitive control. Learn Mem 24:267-277.
Jain C, Belasco J G (1996) A structural model for the HIV-1 rev-RRE complex deduced from altered-specificity rev variants isolated by a rapid ge-netic strategy. Cell 87:115-125.
Joseph F G, Lammie G A, Scolding N J (2007) CNS lupus: a study of 41 patients. Neurology 69:644-654.
Junghans R P, Anderson C L (1996) The protection receptor for IgG catabolism is the 02-microglobulin-containing neonatal intestinal transport re-ceptor. Proc Natl Acad Sci USA 93:5512-5516.
Karaduman R, Fabrizio P, Hartmuth K, Urlaub H, Lu'hrmann R (2006) RNA structure and RNA-protein interactions in purified yeast U6 snRNPs. J Mol Biol 356:1248-1262.
Kazim S F, Chuang S C, Zhao W, Wong R K, Bianchi R, Iqbal K (2017) Early onset network hyperexcitability in presymptomatic Alzheimer's disease transgenic mice is suppressed by passive immunization with anti-human APP/Abeta antibody and by mGluR5 blockade. Front Aging Neurosci 9:71.
Khanam T, Rozhdestvensky T S, Bundman M, Galiveti C R, Handel S, Sukonina V, Jordan U, Brosius J, Skryabin B V (2007) Two primate-specific small non-protein-coding RNAs in transgenic mice: neuronal expression, subcellular localization and binding partners. Nucleic Acids Res 35: 529-539.
Klein D J, Schmeing T M, Moore P B, Steitz T A (2001) The kink-turn: a new RNA secondary structure motif. EMBO J 20:4214-4221.
Kondrashov A V, Kiefmann M, Ebnet K, Khanam T, Muddashetty R S, Brosius J (2005) Inhibitory effect of naked neural BC1 RNA or BC200 RNA on eukaryotic in vitro translation systems is reversed by poly(A)-binding protein (PABP). J Mol Biol 353:88-103.
Kowal C, DeGiorgio L A, Nakaoka T, Hetherington H, Huerta P T, Diamond B, Volpe B T (2004) Cognition and immunity; antibody impairs mem-ory. Immunity 21:179-188.
Kowal C, Degiorgio L A, Lee J Y, Edgar M A, Huerta P T, Volpe B T, Diamond B (2006) Human lupus autoantibodies against NMDA receptors mediate cognitive impairment. Proc Natl Acad Sci USA 103:19854-19859.
Lamon E W, Bennett J C (1970) Antibodies to ribosomal ribonucleic acid (rRNA) in patients with systemic lupus erythematosus (SLE). Immunol-ogy 19:439-442.
Leontis N B, Lescoute A, Westhof E (2006) The building blocks and motifs of RNA architecture. Curr Opin Struct Biol 16:279-287.
Lescoute A, Leontis N B, Massire C, Westhof E (2005) Recurrent structural RNA motifs, Isostericity Matrices and sequence alignments. Nucleic Ac-ids Res 33:2395-2409.
Lin D, Pestova T V, Hellen C U, Tiedge H (2008) Translational control by a small RNA: dendritic BC1 RNA targets the eukaryotic initiation factor 4A helicase mechanism. Mol Cell Biol 28:3008 -3019.
Lin Y, Brosius J, Tiedge H (2001) Neuronal BC1 RNA: co-expression with growth-associated protein-43 messenger RNA. Neuroscience 103:465-479.
Mader S, Brimberg L, Diamond B (2017) The role of brain-reactive autoantibodies in brain pathology and cognitive impairment. Front Immunol 8:1101.
Mikdashi J, Krumholz A, Handwerger B (2005) Factors at diagnosis predict subsequent occurrence of seizures in systemic lupus erythematosus. Neu-rology 64:2102-2107.
Muddashetty R, Khanam T, Kondrashov A, Bundman M, Iacoangeli A, Kremerskothen J, Duning K, Barnekow A, Hüttenhofer A, Tiedge H, Brosius J (2002) Poly(A)-binding protein is associated with neuronal BC1 and BC200 ribonucleoprotein particles. J Mol Biol 321:433-445.
Munro T P, Magee R J, Kidd G J, Carson J H, Barbarese E, Smith L M, Smith R (1999) Mutational analysis of a heterogeneous nuclear ribonucleopro-tein A2 response element for RNA trafficking. J Biol Chem 274: 34389-34395.
Mus E, Hof P R, Tiedge H (2007) Dendritic BC200 RNA in aging and in Alzheimer's disease. Proc Natl Acad Sci USA 104:10679-10684.
Muslimov I A, Santi E, Homel P, Perini S, Higgins D, Tiedge H (1997) RNA transport in dendrites: a cis-acting targeting element is contained within neuronal BC1 RNA. J Neurosci 17:4722-4733.
Muslimov I A, Nimmrich V, Hernandez A I, Tcherepanov A, Sacktor T C, Tiedge H (2004) Dendritic transport and localization of protein kinase MmRNA: implications for molecular memory consolidation. J Biol Chem 279:52613-52622.
Muslimov I A, Iacoangeli A, Brosius J, Tiedge H (2006) Spatial codes in dendritic BC1 RNA. J Cell Biol 175:427-439.
Muslimov I A, Patel M V, Rose A, Tiedge H (2011) Spatial code recognition in neuronal RNA targeting: role of RNA-hnRNP A2 interactions. J Cell Biol 194:441-457.
Muslimov I A, Tuzhilin A, Tang T H, Wong R K, Bianchi R, Tiedge H (2014) Interactions of noncanonical motifs with hnRNP A2 promote activity-dependent RNA transport in neurons. J Cell Biol 205:493-510.
Muslimov I A, Eom T, Iacoangeli A, Chuang S C, Hukema R K, Willemsen R, Stefanov D G, Wong R K S, Tiedge H (2018) BC RNA mislocalization in the fragile X premutation. eNeuro 5:pii: ENEURO.0091-18.2018.
Noller H F (2005) RNA structure: reading the ribosome. Science 309:1508-1514.
Oostra B A, Willemsen R (2009) FMR1: a gene with three faces. Biochim Biophys Acta 1790:467-477.
Popescu A, Kao A H (2011) Neuropsychiatric systemic lupus erythematosus. Curr Neuropharmacol 9:449-457.
Qiao S W, Kobayashi K, Johansen F E, Sollid L M, Andersen J T, Milford E, Roopenian D C, Lencer W I, Blumberg R S (2008) Dependence of antibody-mediated presentation of antigen on FcRn. Proc Natl Acad Sci USA 105:9337-9342.
Rhodes D A, Isenberg D A (2017) TRIM21 and the function of antibodies inside cells. Trends Immunol 38:916-926.
Robeck T, Skryabin B V, Rozhdestvensky T S, Skryabin A B, Brosius J (2016) BC1 RNA motifs required for dendritic transport in vivo. Sci Rep 6:28300.
Rozhdestvensky T S, Kopylov A M, Brosius J, Hüttenhofer A (2001) Neuronal BC1 RNA structure: evolutionary conversion of a tRNA Ala domain into an extended stem-loop structure. RNA 7:722-730.
Schoch A, Thorey I S, Engert J, Winter G, Emrich T (2014) Comparison of the lateral tail vein and the retro-orbital venous sinus routes of antibody administration in pharmacokinetic studies. Lab Anim 43:95-99.
Schur P H, Monroe M (1969) Antibodies to ribonucleic acid in systemic lupus erythematosus. Proc Natl Acad Sci USA 63:1108-1112.
Skryabin B V, Kremerskothen J, Vassilacopoulou D, Disotell T R, Kapitonov V V, Jurka J, Brosius J (1998) The BC200 RNA gene and its neural ex-pression are conserved in Anthropoidea (Primates). J Mol Evol 47: 677-685.
Sturgill B C, Carpenter R R (1965) Antibody to ribosomes in systemic lupus erythematosus. Arthritis Rheum 8:213-218.
Suzuki T, Ishii-Watabe A, Tada M, Kobayashi T, Kanayasu-Toyoda T, Kawanishi T, Yamaguchi T (2010) Importance of neonatal FcR in regulating the serum half-life of therapeutic proteins containing the fc domain of human IgG1: a comparative study of the affinity of monoclonal antibod-ies and fc-fusion proteins to human neonatal FcR. J Immunol 184: 1968-1976.
Talal N (1973) Antibodies binding 3H-reovirus RNA in systemic lupus erythematosus. Clin Immunol Immunopathol 1:230-233.
Tan E M, Cohen A S, Fries J F, Masi A T, McShane D J, Rothfield N F, Schaller J G, Talal N, Winchester R J (1982) The 1982 revised criteria for the classification of systemic lupus erythematosus. Arthritis Rheum 25: 1271-1277.
Tiedge H (1991) The use of UV light as a cross-linking agent for cells and tissue sections in in situ hybridization. DNA Cell Biol 10:143-147.
Tiedge H, Fremeau R T Jr, Weinstock P H, Arancio O, Brosius J (1991) Dendritic location of neural BC1 RNA. Proc Natl Acad Sci USA 88: 2093-2097.
Tiedge H, Chen W, Brosius J (1993) Primary structure, neural-specific expression, and dendritic location of human BC200 RNA. J Neurosci 13: 2382-2390.
Uccellini M B, Busto P, Debatis M, Marshak-Rothstein A, Viglianti G A (2012) Selective binding of anti-DNA antibodies to native dsDNA frag-ments of differing sequence. Immunol Lett 143:85-91.
Uchiumi T, Traut R R, Elkon K, Kominami R (1991) A human autoantibody specific for a unique conserved region of 28 S ribosomal RNA inhibits the interaction of elongation factors 1 alpha and 2 with ribosomes. J Biol Chem 266:2054-2062.
van Venrooij W J, Hoet R, Castrop J, Hageman B, Mattaj I W, van de Putte L B (1990) Anti-(U1) small nuclear RNA antibodies in anti-small nuclear ribonucleoprotein sera from patients with connective tissue diseases. J Clin Invest 86:2154-2160.
Vidovic I, Nottrott S, Hartmuth K, Lührmann R, Ficner R (2000) Crystal structure of the spliceosomal 15.5kD protein bound to a U4 snRNA frag-ment. Mol Cell 6:1331-1342.
Wang H, Iacoangeli A, Popp S, Muslimov I A, Imataka H, Sonenberg N, Lomakin I B, Tiedge H (2002) Dendritic BC1 RNA: functional role in regulation of translation initiation. J Neurosci 22:10232-10241.
Wang H, Iacoangeli A, Lin D, Williams K, Denman R B, Hellen C U, Tiedge H (2005) Dendritic BC1 RNA in translational control mechanisms. J Cell Biol 171:811-821.
Zhong J, Chuang S C, Bianchi R, Zhao W, Lee H, Fenton A A, Wong R K, Tiedge H (2009) BC1 regulation of metabotropic glutamate receptor-mediated neuronal excitability. J Neurosci 29:9977-9986.
Zhong J, Chuang S C, Bianchi R, Zhao W, Paul G, Thakkar P, Liu D, Fenton A A, Wong R K, Tiedge H (2010) Regulatory BC1 RNA and the fragile X mental retardation protein: convergent functionality in brain. PLoS One 5:e15509.
Zirkzee E J, Steup-Beekman G M, van der Mast R C, Bollen E L, van der Wee N J Baptist E, Slee T M, Huisman M V, Middelkoop H A, Luyendijk J, van Buchem M A, Huizinga T W (2012) Prospective study of clinical pheno-types in neuropsychiatric systemic lupus erythematosus; multidisci-plinary approach to diagnosis and therapy. J Rheumatol 39:2118-2126.

### Materials and Methods for Examples 2-4

Mice. A mouse model will be employed to address the question whether lupus anti-BC abs cause BC RNA mislocalization and lupus-like phenotypic alterations in vivo. We will treat mice with LPS to permeabilize the blood-brain barrier and introduce purified SLE anti-BC IgGs by intravenous injection [3,40,68,81]. Mice will be injected i.v. using the retro-orbital route [40,70]. Purified IgGs will be injected (0.5 mg/ml in 100 μl saline) on day 1. 15 min after the i.v. injection, LPS (3 mg/kg) will be injected i.p. LPS i.p. injections will be repeated two days after the i.v. injection.

Animals will be euthanized 2 days later. We will examine RNA and protein localization and susceptibility to prolonged epileptiform discharges and seizures [30,38]. Animal age will be 18-20 days. Both male and female mice will be used. We typically work with mixed-background animals (C57BL/6J and 129×1/SvJ) as such mice have been used in previous work directed at hyperexcitability in the form of prolonged discharges and seizures [30,31]. In addition, BALB/c mice will be used to be able to cross-compare with other previous work [68]. We will use 8 males and 8 females in both the experimental (SLE anti-BC IgG) and control (non-SLE IgG or SLE non-anti-BC IgG) groups for each of the above experimental approaches.

The estimated total number of animals will be 96 for any one type of SLE anti-BC IgG. We have 18 such anti-BC IgGs with high reactivity. BC200 decoys will be i.v. injected to establish whether intercepting SLE anti-BC abs in the circulation will mitigate intracellular mislocalization and neuronal hyperexcitability, including seizures. Animal numbers for these experiments will correspond to those discussed above.

In a second approach, we will immunize mice (age 6 weeks) with human BC200 RNA using SRP9/14 as a carrier (decorated with anti-SRP9 or anti-SRP14 antibodies and complexed with microspheres). Both male and female mice will be used, 20 animals each. The animals will again be on a mixed C57BL/6J and 129×1/SvJ genetic background, consistent with previous publications [30,31,34] (see ref. for a discussion of genetic background preference for phenotype analyses). The immunogen will be injected i.p., using Complete Freunds Adjuvant CFA) for the first injection and Incomplete Freunds Adjuvant (IFA) for booster injections [81,84]. For the first injection (CFA), we will also consider the subcutaneous route (6-point dorsal) which has given us excellent results in previous work [84]. A first booster injection (IFA) will be given two weeks after the first injection (CFA), a second booster injection two weeks after the first. At this time, anti-BC antibody titers will be established by ELISA [84]. We will perform serial dilutions of serum (blood obtained via tail vein) and compare titers with preimmune serum titers. Successfully immunized animals will receive a final booster injection, two weeks after the previous one. This immunization protocol will analogously be applied, using the same number of animals, if the phosphorothioate end-protected 120-nt 5′ segment of BC200 RNA is used as an immunogen. Successfully immunized animals will be examined for RNA localization and cognitive performance [34,38,40].

Rats. Rats (Sprague-Dawley) will be used to generate neurons in primary culture for RNA transport assays. Embryos will be obtained from timed pregnant female animals. Based on past experience, we estimate that we will need about 36 litters per year for the proposed work. One litter is needed for one set of primary cultures.

Mice and rats are standard model systems in the study of brain function. Mice have previously been used for i.v. injection to study the impact of autoimmune antibodies on brain function [3,40,68,81].

Human Subjects. We will work with human subjects to collect blood samples from lupus patients longitudinally. Contact with human subjects in this project will involve drawing of blood samples. This procedure will be preceded by a description of the study to the participant, obtaining assurance of participant understanding, and signing of the IRB-approved consent form.

The enrollment criterion will be SLE. We will not recruit members of vulnerable populations. Statistical consultation (including power analysis, data analysis) will be provided. There are no additional sites where human subject research will be performed as part of this project.

From each participant, we will draw 5 ml of blood for identification and analysis of SLE anti-BC abs. Serum will be prepared for analysis, and samples will be coded and stored in a locked −80° C. freezer. Results will become part of the participants individually identifiable health information (IIHI). We will also have access to patient records (e.g. age, gender, ethnic/racial profile, medical history) although these data will not be collected specifically for this project. Records will be maintained for three years after completion of the study.

### Example 2

### Lupus Anti-BC Abs: Transport Factor Displacement

We discovered that autoantibodies from a subset of SLE patients are directed against neuroregulatory BCRNAs (SLE anti-BC abs). High-reactivity anti-BC abs were detected in 26% of serum samples obtained from lupus patients. These SLE anti-BC abs engage the 5′ GA motif that serves as a dendritic targeting element (DTE) [16]. SLE anti-BC abs quasi permanently displace RNA transport factors from the GA motif DTE and thereby prevent BC RNA delivery to synaptodendritic sites of function [16]. The data indicate that neuronal regulatory BC RNAs can elicit autoimmune responses that lead to phenotypic manifestations in neuropsychiatric SLE.

Here, we functionally dissect the molecular-cellular mechanism by which SLE anti-BC abs cause intraneuronal mislocalization of BC RNAs and consequential sequelae including seizures and cognitive dysfunction. The knowledge thus gained enables the design of efficacious decoys that intercept and neutralize SLE anti-BC abs and mitigate phenotypic sequelae, thereby developing effective therapeutic interventions.

BC RNA mislocalization in neurons has been implicated in epileptogenic and cognitive manifestations. We can investigate phenotypic alterations that result from synapto-dendritic lack of BC RNAs caused by SLE anti-BC abs. The action of such autoantibodies gives rise to seizure susceptibility and cognitive dysfunction. Effective decoys will intercept pathogenic SLE anti-BC abs and, as a result, to rescue BC RNA neuronal functionality. Elucidating this novel RNA mechanism in neuropsychiatric SLE should overcome significant shortcomings in our understanding of this enigmatic disease.

We advance a novel mechanism of anti-RNA autoimmunity in SLE and its neuropsychiatric manifestations. Two types of lupus autoantibodies that directly impact neuronal function have previously been described: those directed at NMDA-type glutamate receptors [3] and those directed at ribosomal P proteins [17]. Distinct from these autoantibodies, the pathogenic SLE anti-BC abs described here target regulatory BC RNAs and cause their intraneuronal mislocalization. We thus introduce a novel brain specific lupus autoantigen and a novel neuronal lupus autoimmunity mechanism: to our knowledge, this is the first-described example of regulatory neuronal RNAs becoming targets of lupus autoimmune reactivities.

The novel molecular-cellular mechanism emerging from the actions of SLE anti-BC abs is diagrammatically summarized in FIG. 12. BC RNA dendritic targeting element (DTE) architectural motifs, which specify transport to synapto-dendritic sites of function, become targets of SLE anti-BC autoantibodies (FIG. 12, Scene 1). The autoantibodies displace RNA transport factor hnRNP A2 which normally binds to these very DTE motifs (Scene 2). As a consequence, dendritic BC RNA delivery (Scene 3) is compromised. The resulting lack of BC RNAs at the synapse causes dysregulation of local translational control (Scene 4), giving rise to phenotypic alterations including seizure activity and cognitive impairment.

The data in Example 1 indicates that SLE anti-BC abs target structural motifs in BC RNA 5′ domains. Here we dissect SLE anti-BC ab interactions with BC RNA 5′ dendritic targeting elements (DTEs). We can assess whether interactions result in the displacement of RNA transport factors from BC RNA DTEs. Such factors will include hnRNP A2 and in particular Purα, newly introduced here. We posit that both are required for BC RNA dendritic targeting, and that both are displaced from BC RNAs by SLE anti-BC abs. We will work with rodent BC1 RNA but increasingly and predominantly with primate BC200 RNA because of the latter's significance in human disease.

### Experimental Design

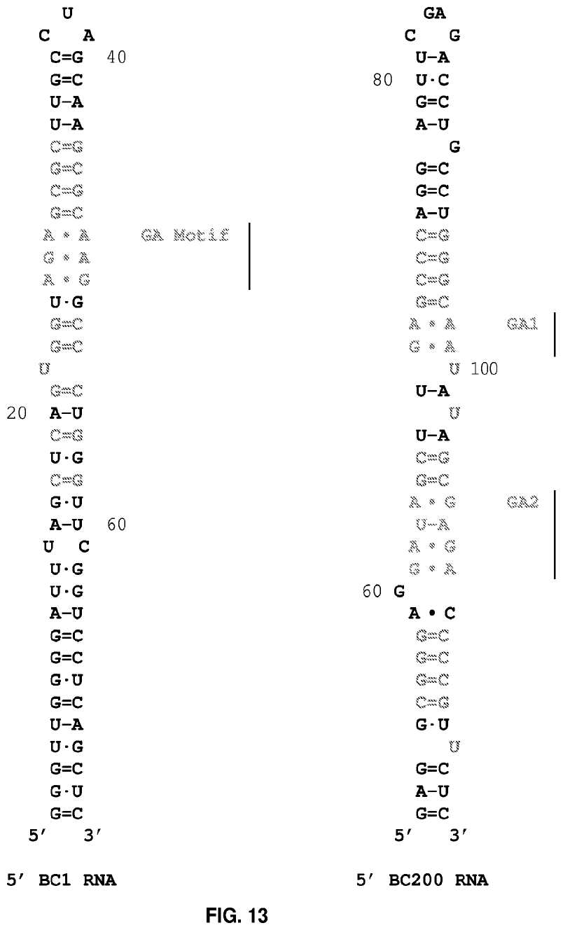

Functional determinants of the human BC200 RNA DTE the target of lupus auto-antibodies can be determined. Structure-function relationships have been analyzed in depth for the rat BC1 RNA DTE [36,37] but not for the human BC200 RNA DTE. Because rodent BC1 RNA and primate BC200 RNA are analogs rather than phylogenetic orthologs (i.e. are of independent evolutionary provenance; refs. [24-26]), functional equivalence can not a priori be assumed. It is therefore important for our understanding of human lupus autoimmunity that the BC200 5′ domain autoantigenic determinants be examined in depth, considering also that modified elements of this domain may prove useful in therapeutic efforts. BC1 and BC200 5′ stem-loop domain secondary structures are shown in FIG. 13.

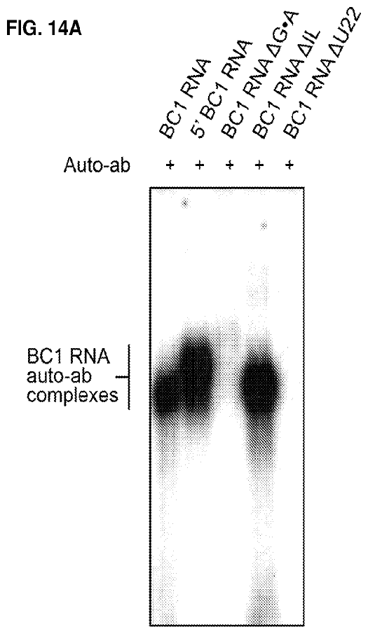

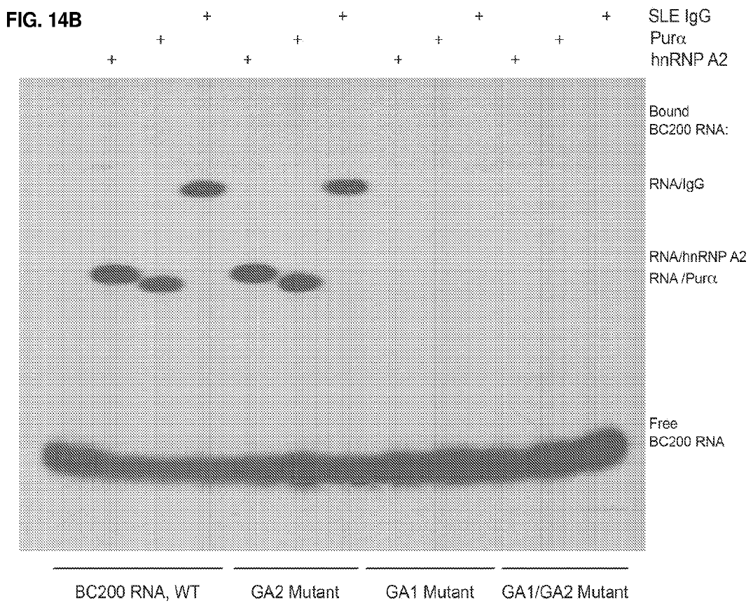

In rat BC1 RNA, elimination of the noncanonical GA motif by conversion of purine·purine pairs to standard Watson-Crick (WC) pairs abolishes dendritic targeting [36,37]. The same conversion also prevents binding of SLE anti-BC IgGs (FIG. 14A). Similarly, deletion of unpaired nucleotide U22, required for dendritic targeting [36,37], also abrogates interaction with SLE anti-BC abs (FIG. 14A). In contrast, conversion of the basal internal loop (IL; U14 C61) to standard WC pairing does not affect binding of SLE anti-BC abs (FIG. 14A). The basal internal loop is not required for dendritic targeting [36,37]. The combined data indicates that SLE anti-BC abs target 5′ domain BC1 RNA DTE structural motifs that specify dendritic delivery.

In human BC200 RNA, the 5′ domain features a stem-loop containing two GA motifs, both with adjacent unpaired U-residues (FIG. 13, refs. [26,41,43,44,46]). The BC200 GA1 motif, but not the BC200 GA2 motif, is required for interactions with RNA transport factors and SLE anti-BC abs (FIG. 14B). The results indicate that transport factors and SLE anti-BC abs recognize the same structural attributes in the BC200 5′ domain. The unpaired U residues will also be examined singly, and in combination. Unpaired U residues will be eliminated or base-paired with A-residues. For each combination of mutations, binding to transport factors and SLE anti-BC abs will be determined. It is noted that in an apparent example of convergent evolution, the noncanonical A·A/G·A pairings are identical in the functionally relevant GA motifs of rat BC1 RNA and human BC200 RNA.

We found that Pura binds to the same BC200 RNA GA1 motif as do hnRNP A2 and lupus anti-BC abs (FIG. 14B). We further found that binding of Pura to BC RNA GA motifs is concurrent with hnRNP A2 binding (i.e. not competitive) and, using siRNA approaches, that Pura is required for BC RNA dendritic targeting (I. A. Muslimov and H. T., unpublished). Displacement of Pura from BC200 RNA by SLE anti-BC abs can also be determined.

We will establish a minimal BC200 DTE, comprising the structural attributes that are necessary and sufficient to specify transport factor binding which are also targeted by SLE anti-BC abs and thereby develop new therapeutic approaches. We have proof-of-principle evidence that the seizure phenotype that is observed after introduction of SLE anti-BC abs into wild-type (WT) mice fails to materialize with the same SLE anti-BC abs coinjected with BC200 RNA or the BC200 5′ domain (Table 2).

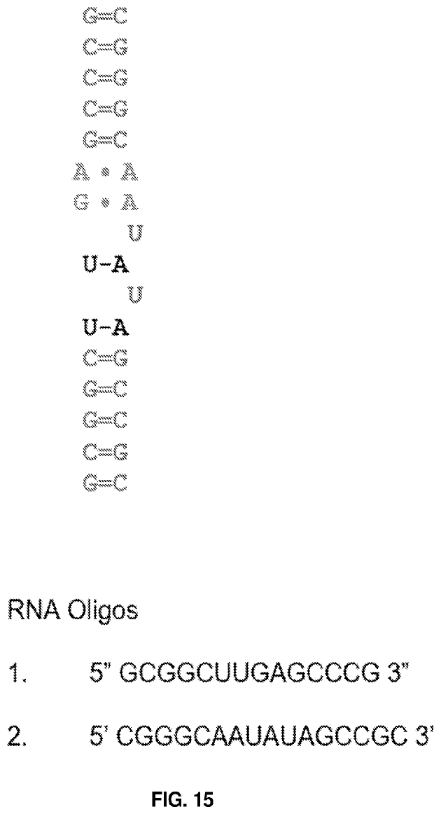

In view of the foregoing, anti-BC200 RNA lupus autoimmune responses can be mitigated by titration with BC200-derived blocking antigens (which are referred to herein as BC200 decoys). We have shown that bound to SLE anti-BC abs, BC200 RNA and the BC200 5′ domain are remarkably stable, with no perceptible degradation or dissociation over at least one week. We will employ BC200 RNA and the BC200 5′ domain as decoys but will in addition use a minimal BC200 decoy that comprises the apical GA motif (GA1) and adjacent unpaired U-residues, flanked by G=C base-pair clamps. A minimal structure is desirable because of administration considerations. Examples of the minimal structure for BC200 Decoys are shown in FIG. 15. For additional long-term molecular stability, the minimal BC200 decoy will be end-protected by phosphorothioate bonds. RNA synthesis and modification will be performed by IBA Lifesciences (Göttingen, Germany), a company that we have come to trust in customized RNA design. Modifications, such as those described above, will also be introduced into the decoy sequence to increase nuclease resistance and stability in vivo. These include without limitation, 2-O-methyl moieties, 2-O-methyoxy, LNA, FANA, UNA, substitution of phosphodiester linkages with phosphorothioate linkages and modified nucleosides which are resistant to 3′ and 5′ exonucleases.

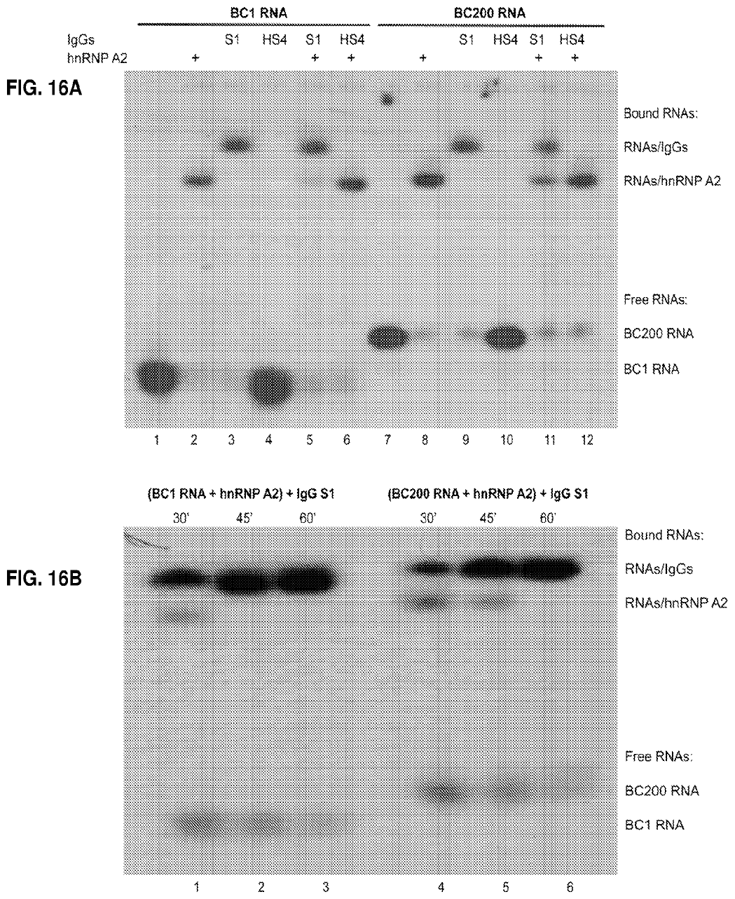

We will thus establish the ability of BC200 decoys to engage SLE anti-BC IgGs. Working with such IgGs in EMSA analysis, we have shown that at equimolar concentrations, they completely displace hnRNP A2 from BC1 and BC200 RNAs (FIG. 16). Such displacement seems to be a defining feature of Group I (Table 1) lupus IgGs. For binding of hnRNP A2 to BC1 RNA, quantitative EMSA analysis has revealed dissociation equilibrium constants (Kd) in the nanomolar range [37]. As neither hnRNP A2 nor Pura is able to displace SLE anti-BC IgGs from BC RNAs because of the IgGs' slow off-rates, we estimate that binding of SLE anti-BC IgGs to BC RNAs can be several orders of magnitude stronger than binding of hnRNP A2 or Purα, and thus essentially irreversible. Such high binding affinities are not unusual for powerful antibodies but would be counter-productive for transport factors that go through cycles of cargo engagement and disengagement.

We use EMSA displacement approaches to confirm the ability of BC200 decoys, following binding to SLE anti-BC IgGs, to prevent those IgGs from subsequently targeting BC RNAs and displacing bound transport factors. As a consequence of the decoy action, BC RNAs will remain free to interact with transport factors hnRNP A2 and Purα, engagements that are essential for dendritic targeting. To verify that binding affinities of BC200 decoys to SLE anti-BC IgGs are similar to that of BC200 RNA, we will use quantitative EMSA analysis to measure dissociation constants [50,51] as we have previously reported [37,52].

To minimize binding by non BC200 specific antibodies, we will subject SLE anti-BC abs to additional affinity purification using BC200 RNA or its 5′ domain coupled to sepharose [53]. Two purposes will be served by this approach. (i) It will provide additional validation that phenotypic manifestations (e.g. epileptogenesis) are attributable exclusively to lupus autoantibodies against BC RNAs. (ii) Flow-through IgGs of the affinity purification can be used as negative controls in the assessment of phenotypic manifestations.

### Example 3

### BC RNA Mislocalization

Displacement of RNA transport factors hnRNP A2 and Pura from BC RNA dendritic targeting elements by SLE anti-BC abs negatively impacts BC RNA dendritic transport and localization in neurons.

We have dissected molecular interactions of SLE anti-BC abs with BC RNA dendritic targeting element motif structures, consequential displacement of transport factors hnRNP A2 and Pura from such structures, and rescue of transport factor-DTE interactions through application of BC200 decoys. Here, we will investigate resulting impairments of BC RNA intraneuronal transport and localization. Specifically, we will (i) elucidate the impact of lupus anti-BC abs on BC RNA dendritic transport in neurons in primary culture and on the localization of endogenous BC1 RNA in vivo, and (ii) examine BC200 decoy-mitigation of BC RNA transport and localization impairments induced by SLE anti-BC abs.

### Experimental Design

We will work with neurons in primary culture to establish the impact of SLE anti-BC abs on dendritic BC RNA transport. We will use microinjection techniques to introduce RNAs, including decoys, into cultured neurons (typically sympathetic or hippocampal), using established methodologies [35-37,52,57-61]. This approach allows precise control of RNA amounts introduced, such that the number of injected RNA molecules is significantly lower than the number of respective endogenous RNA molecules [35-37,52]. In certain embodiments we will work with radiolabeled RNAs as GA motifs are intolerant of modifications (e.g. fluorescent or other side chains) that may disrupt motif structure [37]. Using this experimental approach, we have shown that the BC1 5′ stem-loop domain with its resident GA motif and unpaired U22 is targeting-determinant [35-37]. A BC1 RNA 5′ DTE requirement for dendritic localization has been confirmed using transgenic mice (ref. [62]). We have applied the above techniques to measure RNA transport, and we have developed methodology to quantify dendritic RNA transport both spatially and temporally [35-37,52,58,63]. Using time course experiments and nonlinear regression analysis, we have established anterograde dendritic translocation of BC1 RNA at a velocity of 388±15 μm/h [35].

We have found that phenotypic manifestations (i.e. seizures) that are elicited by introduction of lupus anti-BC abs in wild-type animals are prevented by application of such decoys. As we hypothesize that these phenotypic alterations are caused by lack of BC RNAs at the synapse (FIG. 12), we predict that dendritic transport impairments caused by lupus anti-BC IgGs will be alleviated by BC200 decoys. We will bath-coapply BC200 decoys with SLE anti-BC IgGs to titrate the molar ratio sufficient to achieve significant dendritic delivery rescue of subsequently microinjected BC RNAs, and we will use immunocytochemistry directed at human IgG to determine that application of decoy prevents cellular uptake of SLE IgGs. We will also perform sequential injections (BC200 decoy preceding radiolabeled BC RNAs) to establish the optimal time differential for the decoy to be able to intercept bath-applied SLE anti-BC IgGs and rescue dendritic targeting.

In a related effort, we will increase intracellular concentrations of transport factors hnRNP A2 and Pura to alleviate transport deficiencies caused by SLE anti-BC IgGs, that results in displacement of these transport factors. For this experiment, we will coinject recombinant hnRNP A2 and Pura with radiolabeled BC RNAs. We have previously documented the feasibility of this approach by demonstrating that BC RNA dendritic delivery, curtailed by competition of CGG-repeat RNA for access to hnRNP A2, can be significantly restored by the intracellular introduction of hnRNP A2 [37].

The concept of transport inhibition by binding of lupus anti-BC IgGs to BC RNA dendritic targeting elements indicates that such SLE IgGs would, to a substantial degree, intracellularly colocalize with BC RNAs in a somatic perinuclear fashion, similar to what is exemplified by the doughnut-shaped localization signal of microinjected BC1 RNA in FIG. 9C.

We will also determine whether in vivo, sufficient amounts of SLE anti-BC abs enter the CNS and are taken up by neurons to cause mislocalization of endogenous BC1 RNA in mice. We will treat mice with lipopolysaccharide (LPS) to temporarily permeabilize the blood-brain barrier (BBB) and deliver purified SLE anti-BC IgGs by intravenous injection [40,68], using the retro-orbital route [70]. This combined experimental strategy has been established in our lab for the purpose of introducing SLE anti-BC abs into CNS neurons in vivo, and we have documented that SLE anti-BC abs thus administered are active intraneuronally [40].

Ascertaining BC1 RNA mislocalization in stratified brain areas, we show (FIG. 19) that in hippocampal CA1, SLE anti-BC abs but not non-SLE abs significantly reduce localization of endogenous BC1 RNA to synapto-dendritic strata oriens and radiatum and significantly increase BC1 RNA localization to somatic stratum pyramidale, with the increase in the somatic stratum matching the decrease in dendritic strata (FIG. 9E, 9F). The data indicate that after entering the brain and being taken up by neurons, SLE anti-BC abs target BC1 RNA, interfering with its anterograde delivery to synapto-dendritic domains and thus causing it to accumulate in neuronal somata. Overall, the results show that our in vivo approach is practicable.

Going forward, the efficacy of BC200 decoys in rescuing dendritic localization of endogenous BC1 RNA in vivo will be demonstrated. We will intravenously coinject, or sequentially inject, BC200 decoys and SLE anti-BC IgGs at a range of molar ratios to titrate localization rescue efficiency. In this approach, BC200 decoys in the circulation will be engaged by SLE anti-BC IgGs. The resulting complexes are stable because of high binding affinities. Thus, if taken up by neurons (to be assessed by immunohistochemistry directed at human IgG [68]), they will be unable to target endogenous BC1 RNA which will therefore be free to interact with transport factors. In addition, extracellular BC200 decoy-lupus anti-BC IgG immune complexes can be taken up for clearance by Fc receptor-expressing glial cells [64,71,72]. BC1 RNA localization rescue will be quantified by in situ hybridization as described (FIG. 9E,9F).

In the KO model mouse, lack of BC1 RNA, a translational repressor in the basal default state [27-29], causes overexpression of synaptic proteins, including PSD-95 and a number of glutamate receptors (GluRs) [30,32]. We hypothesize that synapto-dendritic lack of BC RNAs will analogously result in excessive translation of local mRNAs that are normally under BC RNA control. We will perform immunocytochemistry with hippocampal neurons in primary culture [69,73] to ascertain synapto-dendritic levels of the above proteins following bath application of lupus anti-BC abs. We will also work with cortical synaptoneurosome preparations (using the Bassell protocol [74]) from SLE IgG-injected mice vs. control injected mice, quantifying differential levels of PSD-95 and GluRs by Western analysis, to document excessive local protein synthesis in BC1 KO mice [32]. As we have shown (Table 2) that the hyper-excitability phenotype of SLE anti-BC IgG-injected mice is more severe than that of BC1 KO mice, we are confident that increased levels of PSD-95 and GluRs in the former can be readily detected. To measure translation rather than levels of synthesized proteins in synaptoneurosome preparations, we will use the chloramphenicol acetyltransferase (CAT) mRNA reporter system. We have previously used CAT mRNA for this purpose as it features a complex 5′ UTR that makes it a target of BC RNA translational control [27,28].

FIGS. 17 and 18 document BC RNA mislocalization, caused by SLE anti-BC abs, in sympathetic and hippocampal neurons, respectively. In vivo, mislocalization appears to be common to BC1-expressing neurons, judging from in situ hybridization-probed brain sections obtained after SLE anti-BC ab i.v. injections. As audiogenic seizures are induced by SLE anti-BC abs, future in situ work will in particular also scrutinize BC RNA localization dysregulation in the auditory system, including DCIC neuropil and AVCN large bushy cells where significant amounts of BC1 RNA are expressed and localized in WT animals [75].

As mentioned above, in a type of structural equivalence known as molecular mimicry [3], BC RNA DTE noncanonical GA motifs resemble structures in viral genomic RNAs or mRNAs and may therefore be confused with non-self by the immune system [3,40]. RNA noncanonical motifs with A·A, A·G, G·A, and G·G content are found, for example, in stem-loop IIb of HIV Rev response element and in the 3′ UTR of SARS-COV-2 RNA [77]. An anti-viral RNA immune response may therefore have the potential to elicit significant and persistent reactivity against BC RNAs. We will identify such viral RNA motifs as targets of SLE anti-BC abs. We will also use an algorithm to screen for analogous noncanonical motifs in viral RNAs other than the above, and will test positive candidates for recognition by SLE anti-BC abs.

### Example 4

### Lupus Anti-BC ABS: Phenotypic Manifestations

Our data indicate that lupus IgG-induced BC RNA mislocalization is associated with consequential phenotypic alterations, including epileptogenesis and cognitive dysfunction. We further submit the hypothesis that in lupus patients, anti-BC ab serum levels fluctuate with disease status (active disease, remission, relapse) and neurological manifestations.

### Experimental Design

We will use two approaches directed at hyperexcitability (in both of which we have significant experience; refs. [30,31,38]). (i) To probe susceptibility to prolonged epileptiform discharges, we will perform intracellular recordings from CA3 pyramidal cells in hippocampal slice preparations. To elicit discharges, we trigger synaptic release of glutamate using the GABAA receptor antagonist bicuculline, as described [30,31,38]. Prolonged epileptiform discharges were thus elicited in BC1 KO animal preparations but not in WT animal preparations [30]. We will confirm that similar epileptiform discharges will be observed in hippocampal slice preparations from WT animals injected with SLE anti-BC abs from Group I patients (Table 1).

(ii) In the second approach, we will assess audiogenic seizure susceptibility as described [30,31,38]. We have recently established that high-reactivity Group I lupus anti-BC sera or purified IgGs (Table 1), i.v. injected using the above injection routine, cause acute responses to auditory stimulation in WT mice. Immediate-onset, severe generalized seizures of the clonic-tonic type were observed in all cases with a mortality of 100% for purified lupus anti-BC IgGs (Table 2). When BC200 decoys were i.v. coinjected with lupus sera or lupus IgGs (the latter at decoy:IgG ratios of 1:1), audiogenic seizures did not materialize.

TABLE 2
Seizure susceptibility and mortality of wild-type mice
injected with SLE or non-SLE sera or purified IgGs.

| Group | N | Seizures (%) | Mortality (%) |
|---|---|---|---|
| SLE sera | 19 | 100 | 74 |
| SLE sera + decoys | 7 | 0 | 0 |
| Non-SLE sera | 8 | 0 | 0 |
| SLE IgGs | 12 | 100 | 100 |
| SLE IgGs + decoys | 12 | 0 | 0 |
| Non-SLE IgGs | 10 | 0 | 0 |

*SLE samples were from 5 patients, non-SLE samples were from healthy subjects and patients with rheumatoid arthritis or multiple sclerosis.
N, number of mice.

We have thus established a robust experimental system to assess the epileptogenic potential of lupus sera and IgGs. The rescue data with BC200 decoys indicate that it is indeed lupus anti-BC RNA autoimmunity that is responsible for the observed seizure phenotype. Seizures are a common but poorly understood manifestation in lupus [5,6], and our work identifies anti-BC200 reactivity as a cause. The data indicate therapeutic intervention utility of BC200 decoys for neuropsychiatric lupus patients with seizures.

We will now use our i.v. injection approach to examine BC200 decoy efficiency with the lupus autoantibodies of Table 1 Group 1. We will titrate the amounts of BC200 decoys (molar ratios to lupus IgGs) required to prevent seizures. We will establish the time course of BC200 decoy effectiveness in a series of two-step sequential decoy/IgG i.v. injections (i.e. left eye, right eye). We will interval-bleed injected mice to establish biological half-lives of IgG-bound circulating decoys. We will use the decoys, including the minimal BC200 decoy that lacks the 3′ C-loop motif domain and will therefore not be able to impact translational control. To verify that BC200 decoys do not cause side effects, they will be i.v. injected into naïve animals which will subsequently undergo comprehensive health checks [83]. We trust that these efforts will also encourage initial steps towards future clinical trial efforts with neuropsychiatric lupus patients.

Cognitive dysfunction will be investigated using the Attentional Set Shift Task (ASST) paradigm, as described [34,38]. We will also raise antibodies directed at human BC200 RNA in mice. DTE-resident GA motifs are (auto) immunogenic because of their similarity to viral structures [40]. Jung et al. raised a monoclonal anti-BC200 RNA antibody which targets GA motif GA1 (FIG. 13). Our work has shown that lupus anti-BC200 RNA abs also recognize the 5′ GA motif of rodent BC1 RNA [40].

Immunization and BBB permeabilization will be performed following the Volpe/Diamond protocol [68,81], informed also by own experience in this area [40,84]. BC RNAs are exceptionally stable as their 5′ and 3′ ends are protected by double-stranded stem-loop structures [28,36,42]. We will work with a 120-nt 5′ segment of BC200 RNA, reinforced by phosphorothioate bonds as described herein. (The 3′ and central segments are not immunogenic [40,43].) The 120-nt 5′ segment contains a pseudoknot structure and the dendritic targeting element stem-loop domain with resident GA1. For immunization, we will complex the 5′ segment with signal recognition particle (SRP) protein heterodimer 9/14. SRP9/14 avidly binds to a pseudoknot structure in SRP RNA and, because of structural similarity, also to the 5′ segment of primate BC200 RNA (but not to rodent BC1 RNA) [85-87]. The Kd is in the picomolar range, indicating essentially irreversible binding ([87). While binding to the BC200 RNA pseudoknot structure, SRP9/14 does not interact with the BC200 RNA DTE structure including GA1. Complexing BC200 RNA with SRP9/14 will promote an anti-BC200 RNA immune response as SRP9/14 will act as a carrier (activating helper T cells) for the hapten, the BC200 5′ segment. Recombinant human SRP9/14 will be generated as described [87]. It will be decorated with anti-SRP9 and anti-SRP14 antibodies (commercially available) that are complexed with sub-μm microspheres [88].

Mice will be immunized by i.p. injection of the BC200 5′ segment complexed as described above. Complete Freund's Adjuvant will be used for the first injection; Incomplete Freund's Adjuvant for three booster injections to be given in two-week intervals [81,84]. Anti-BC ab titers will be established by ELISA [81,84]. We will identify BC200 and BC1 RNA structural motifs that are targeted by purified anti-BC abs from immunized mice and, anticipating they include DTE-resident GA motifs, examine displacement of transport factors hnRNP A2 and Purα. Following LPS-induced BBB permeabilization of immunized mice [40,68,81], mislocalization of endogenous BC1 RNA will be examined, and synapto-dendritic overexpression of PSD-95 and GluRs in brain will be assessed as described herein.

BC1 RNA model animals that lack BC1 RNA globally or synaptically are significantly impaired in their cognitive competence [34,38]. Cognitive flexibility, a form of cognitive competence mediated by the prefrontal cortex [89,90], is compromised in such animals. Their behavior continues to be dominated by previously acquired memories even if newly changed external contingencies are now in conflict with recall of such memories [34,38]. This type of cognitive dysfunction has been [34,38] and will be examined using the Attentional Set Shift Task (ASST). The ASST is a robust behavioral routine that is uniquely suited to probe prefrontal cortical function and cognitive flexibility [34,38,89-91]. We will use the ASST to assess cognitive impairment resulting from BC RNA mislocalization caused by anti-BC abs.

Excessive self-grooming and deficiencies in hippocampus-dependent active place avoidance learning will be examined as further manifestations of cognitive dysfunction, as has previously been described for BC1 RNA model animals [31,33,34,38]. Behavioral testing of immunized mice will be preceded by LPS injections for BBB permeabilization or sham injections for controls [40,68,81]. Lupus autoantibodies may also cause neuronal cell degeneration and death, be it directly or by triggering seizure activity [3,92]. We will therefore assess cell damage and loss in brains of immunized mice, using Nissl staining, detection of activated caspase-3, and fluoro-jade reactivity as described by our collaborators [68,81].

We will conduct a longitudinal study with lupus patients to investigate whether the strength of anti-BC autoimmune reactivity is reflected in lupus disease status, including neuropsychiatric manifestations such as seizures. We plan to establish, by following lupus patients during surveillance and therapy, whether monitoring anti-BC ab titers is useful in treatment outcome assessment and prediction.

IgG will be purified from patient sera as described [40]. ELISA and quantitative EMSA analysis [37,52,81,84] will be performed to establish SLE anti-BC reactivity and hnRNP A2/Purα displacement. Spearman's rank-order correlation analysis will be used to quantify the link between SLE anti-BC reactivity and occurrence of neuropsychiatric manifestations. On the basis of the data shown in Table 1, we expect that about 26% of SLE patients will present with high anti-BC reactivity. Blood will be collected from lupus patients every six months and every time they come to the clinic (for checkup or treatment, e.g. because of seizures or other adverse events). As serum levels of total IgG may vary with disease activity, normalization to total IgG will be used to establish SLE anti-BC IgG titers over time.

In addition to Spearman's analysis, we will use generalized linear mixed model methodology with SLE anti-BC ab titers as a predictor and disease status as an outcome. This approach will account for correlations between measurements from the same and different individuals in each group. To examine the discriminative power of SLE anti-BC abs as potential biomarkers, we will apply Receiver Operating Characteristics (ROC) analysis [93].

We will confirm that increased serum levels of SLE anti-BC IgGs will be accompanied by an exacerbation of lupus manifestations while decreased levels will coincide with remission and clinical stability. Monitoring of SLE anti-BC ab titers may thus help provide outcome indicators for lupus patients with neurological manifestations, in particular seizures.

The BC200 decoys described herein will be administered via both i.v. injection and via intraventricular infusion. This approach has been successfully applied with limited-size constructs including antisense oligonucleotides [48]. We will thus be able to introduce BC200 decoys on either side of the blood-brain barrier.

Human SRP9/14, or anti-SRP9/14 antibodies, which may trigger immune responses in certain mice. Such animals, identified by ELISA, will be excluded from analysis. SRP RNA, which binds SRP9/14 but lacks a dendritic targeting element, is not delivered to dendrites and is not recognized by anti-BC200 RNA abs [43].

Increased BBB permeability has been reported for SLE patients [96]. As such, our samples include sera and cerebrospinal fluid from the same patients, and they will be provided together with data on disease status and neuropsychiatric manifestations. We will thus be able to arrive at a differential appreciation of anti-BC reactivity and BBB permeability in the causation of neuropsychiatric alterations in lupus patients.

### REFERENCES FOR EXAMPLES 2-4

1. Popescu A, Kao A H (2011) Neuropsychiatric systemic lupus erythematosus. Curr Neuropharmacol 9:449-457.
2. Zirkzee E J, Steup-Beekman G M, van der Mast R C, Bollen E L, van der Wee N J, Baptist E, Slee T M, Huisman M V, Middelkoop H A, Luyendijk J, van Buchem M A, Huizinga T W
(2012) Prospective study of clinical phenotypes in neuropsychiatric systemic lupus erythematosus; multidisciplinary approach to diagnosis and therapy. J Rheumatol 39:2118-2126.
3. Mader S, Brimberg L, Diamond B (2017) The role of brain-reactive autoantibodies in brain pathology and cognitive impairment. Front Immunol 8:1101.
4. Mikdashi J, Krumholz A, Handwerger B (2005) Factors at diagnosis predict subsequent occurrence of seizures in systemic lupus erythematosus. Neurology 64:2102-2107.
5. Cimaz R, Guerrini R (2008) Epilepsy in lupus. Lupus 17:777-779.
6. Hanly J G (2019) Overview of neuropsychiatric SLE: current status and future directions. ACR/ARP Annual Meeting, Atlanta, GA.
7. Arbuckle M R, McClain M T, Rubertone M V, Scofield R H, Dennis G J, James J A, Harley J B (2003) Development of autoantibodies before the clinical onset of systemic lupus erythematosus. N Engl J Med 349:1526-1533.
8. Elkon K, Casali P (2008) Nature and functions of autoantibodies. Nat Clin Pract Rheumatol 4:491-498.
9. Crow M K (2010) Long interspersed nuclear elements (LINE-1): potential triggers of systemic autoimmune disease. Autoimmunity 43:7-16.
10. Barrat F J, Elkon K B, Fitzgerald K A (2016) Importance of nucleic acid recognition in inflammation and autoimmunity. Annu Rev Med 67:323-336.
11. Wolin S (2015) RNPs and autoimmunity: 20 years later. RNA 21:548-549.
12. Schur P H, Monroe M (1969) Antibodies to ribonucleic acid in systemic lupus erythematosus. Proc Natl Acad Sci USA 63:1108-1112.
13. Talal N (1973) Antibodies binding 3H-reovirus RNA in systemic lupus erythematosus.
Clin Immunol Immunopathol 1:230-233.
14. Sturgill B C, Carpenter R R (1965) Antibody to ribosomes in systemic lupus erythematosus. Arthritis Rheum 8:213-218.
15. Lamon E W, Bennett J C (1970) Antibodies to ribosomal ribonucleic acid (rRNA) in patients with systemic lupus erythematosus (SLE). Immunology 19:439-442.
16. Chu J L, Brot N, Weissbach H, Elkon K (1991) Lupus antiribosomal P antisera contain antibodies to a small fragment of 28S rRNA located in the proposed ribosomal GTPase center. J Exp Med 174:507-514.
17. Uchiumi T, Traut R R, Elkon K, Kominami R (1991) A human autoantibody specific for a unique conserved region of 28 S ribosomal RNA inhibits the interaction of elongation factors 1 alpha and 2 with ribosomes. J Biol Chem 266:2054-2062.
18. Elkon K B, Bonfa E, Brot N (1992) Antiribosomal antibodies in systemic lupus erythematosus. Rheum Dis Clin North Am 18:377-390.
19. Noller H F (2005) RNA structure: reading the ribosome. Science 309:1508-1514.
20. Grandin K, ed (2010) The Nobel Prizes 2009 (Nobel Foundation, Stockholm).
21. Deutscher S L, Keene J D (1988) A sequence-specific conformational epitope on U1 RNA is recognized by a unique autoantibody. Proc Natl Acad Sci USA 85:3299-3303.
22. Hoet R M, Pieffers M, Stassen M H, Raats J, de Wildt R, Pruijn G J, van den Hoogen F, van Venrooij W J (1999) The importance of the light chain for the epitope specificity of human anti-U1 small nuclear RNA autoantibodies present in systemic lupus erythematosus patients. J Immunol 163:3304-3312.
23. Van Venrooij W J, Hoet R, Castrop J, Hageman B, Mattaj I W, van de Putte L B (1990)
Anti-(U1) small nuclear RNA antibodies in anti-small nuclear ribonucleoprotein sera from patients with connective tissue diseases. J Clin Invest 86:2154-2160.
24. Iacoangeli A, Tiedge H (2013) Translational control at the synapse: role of RNA regulators. Trends Biochem Sci 38:47-55.
25. Eom T, Muslimov I A, Iacoangeli A, Tiedge H (2018) Dendritic targeting and regulatory RNA control of local neuronal translation. In The Oxford Handbook of Neuronal Protein Synthesis, ed Sossin W (Oxford University Press, New York; published online at oxfordhandbooks.com).
26. Lee Y, Lee H S, Kim M, Shin H (2020) Brain cytoplasmic RNAs in neurons: from biosynthesis to function. Biomolecules 10:313.
27. Lin D, Pestova T V, Hellen C U, Tiedge H (2008) Translational control by a small RNA: dendritic BC1 RNA targets the eukaryotic initiation factor 4A helicase mechanism. Mol Cell Biol 28:3008-3019.
28. Eom T, Berardi V, Zhong J, Risuleo G, Tiedge H (2011) Dual nature of translational control by regulatory BC RNAs. Mol Cell Biol 31:4538-4549.
29. Eom T, Muslimov I A, Tsokas P, Berardi V, Zhong J, Sacktor T C, Tiedge H (2014) Neuronal BC RNAs cooperate with eIF4B to mediate activity-dependent translational control. J Cell Biol 207:237-252.
30. Zhong J, Chuang S C, Bianchi R, Zhao W, Lee H, Fenton A A, Wong R K, Tiedge H (2009) BC1 regulation of metabotropic glutamate receptor-mediated neuronal excitability. J Neurosci 29:9977-9986.
31. Zhong J, Chuang S C, Bianchi R, Zhao W, Paul G, Thakkar P, Liu D, Fenton A A, Wong R K, Tiedge H (2010) Regulatory BC1 RNA and the fragile X mental retardation protein: convergent functionality in brain. PLoS One 5:e15509.
32. Briz V, Restivo L, Pasciuto E, Juczewski K, Mercaldo V, Lo A C, Baatsen P, Gounko N V, Borreca A, Girardi T, et al. (2017) The non-coding RNA BC1 regulates experience-dependent structural plasticity and learning. Nat Commun 8:293.
33. Chung A, Dahan N, Alarcon J M, Fenton A A (2017) Effects of regulatory BC1 RNA deletion on synaptic plasticity, learning, and memory. Learn Mem 24:646-649.
34. Iacoangeli A, Dosunmu A, Eom T, Stefanov D G, Tiedge H (2017) Regulatory BC1 RNA in cognitive control. Learn Mem 24:267-277.
35. Muslimov I A, Santi E, Homel P, Perini S, Higgins D, Tiedge H (1997) RNA transport in dendrites: a cis-acting targeting element is contained within neuronal BC1 RNA. J Neurosci 17:4722-4733.
36. Muslimov I A, Iacoangeli A, Brosius J, Tiedge H (2006) Spatial codes in dendritic BC1 RNA. J Cell Biol 175:427-439.
37. Muslimov I A, Patel M V, Rose A, Tiedge H (2011) Spatial code recognition in neuronal RNA targeting: role of RNA-hnRNP A2 interactions. J Cell Biol 194:441-457.
38. Muslimov I A, Eom T, Iacoangeli A, Chuang S C, Hukema R K, Willemsen R, Stefanov D G, Wong R K S, Tiedge H (2018) BC RNA mislocalization in the fragile X premutation. eNeuro 5.
39. Gonzilez A, Massardo L (2018) Antibodies and the brain: antiribosomal P protein antibody and the clinical effects in patients with systemic lupus erythematosus. Curr Opin Neurol 31:300-305.
40. Muslimov I A, Iacoangeli A, Eom T, Ruiz A, Lee M, Stephenson S, Ginzler E M, Tiedge H (2019) Neuronal BC RNA transport impairments caused by systemic lupus erythematosus autoantibodies. J Neurosci 39:7759-7777.
41. Skryabin B V, Kremerskothen J, Vassilacopoulou D, Disotell T R, Kapitonov V V, Jurka J, Brosius J (1998) The BC200 RNA gene and its neural expression are conserved in Anthropoidea (Primates). J Mol Evol 47:677-685.
42. Rozhdestvensky T S, Kopylov A M, Brosius J, Huttenhofer A (2001) Neuronal BC1 RNA structure: evolutionary conversion of a tRNAAla domain into an extended stem-loop structure.
RNA 7:722-730.
43. Jung E, Lee J, Hong H J, Park I, Lee Y (2014) RNA recognition by a human antibody against brain cytoplasmic 200 RNA. RNA 20:805-814.
44. Booy E P, McRae E K, Howard R, Deo S R, Ariyo E O, Dzananovic E, Meier M, Stetefeld J, McKenna S A (2016) RNA helicase associated with AU-rich element (RHAU/DHX36) interacts with the 3′-tail of the long non-coding RNA BC200 (BCYRN1). J Biol Chem 291:5355-5372.
45. Mladek A, Sharma P, Mitra A, Bhattacharyya D, Sponer J, Sponer J E (2009) Trans Hoogsteen/sugar edge base pairing in RNA. Structures, energies, and stabilities from quantum chemical calculations. J Phys Chem B 113:1743-1755.
46. Jang S, Shin H, Lee J, Kim Y, Bak G, Lee Y (2017) Regulation of BC200 RNA-mediated translation inhibition by hnRNP E1 and E2. FEBS Lett 591:393-405.
47. Ohashi S, Kobayashi S, Omori A, Ohara S, Omae A, Muramatsu T, Li Y, Anzai K (2000) The single-stranded DNA- and RNA-binding proteins Pur a and Pur 0 link BC1 RNA to microtubules through binding to the dendrite-targeting RNA motifs. J Neurochem 75:1781-1790.
48. Hua Y, Sahashi K, Hung G, Rigo F, Passini M A, Bennett C F, Krainer A R (2010) Antisense correction of SMN2 splicing in the CNS rescues necrosis in a type III SMA mouse model. Genes Dev 24:1634-1644.
49. Murphy G, Isenberg D A (2019) New therapies for systemic lupus erythematosus—past imperfect, future tense. Nat Rev Rheumatol 15:403-412.
50. Ryder S P, Recht M I, Williamson J R (2008) Quantitative analysis of protein-RNA interactions by gel mobility shift. Methods Mol Biol 488:99-115.
51. Chao J A, Patskovsky Y, Patel V, Levy M, Almo S C, Singer R H (2010) ZBP1 recognition of beta-actin zipcode induces RNA looping. Genes Dev 24:148-158.
52. Muslimov I A, Tuzhilin A, Tang T H, Wong R K, Bianchi R, Tiedge H (2014) Interactions of noncanonical motifs with hnRNP A2 promote activity-dependent RNA transport in neurons. J Cell Biol 205:493-510.
53. Allerson C R, Martinez A, Yikilmaz E, Rouault T A (2003) A high-capacity RNA affinity column for the purification of human IRP1 and IRP2 overexpressed in Pichia pastoris. RNA 9:364-374.
54. Sosinska-Zawierucha P, Zawierucha P, Breborowicz A, Barciszewski J (2018) Prediction of secondary and tertiary structures of human BC200 RNA (BCYRN1) based on experimental and bioinformatic cross-validation. Biochem J 475:2727-2748.
55. Kremerskothen J, Nettermann M, op de Bekke A, Bachmann M, Brosius J (1998) Identification of human autoantigen La/SS-B as BC1/BC200 RNA-binding protein. DNA Cell Biol 17:751-759.
56. Kondrashov A V, Kiefmann M, Ebnet K, Khanam T, Muddashetty R S, Brosius J (2005) Inhibitory effect of naked neural BC1 RNA or BC200 RNA on eukaryotic in vitro translation systems is reversed by poly(A)-binding protein (PABP). J Mol Biol 353:88-103.
57. Shan J, Munro T P, Barbarese E, Carson J H, Smith R (2003) A molecular mechanism for mRNA trafficking in neuronal dendrites. J Neurosci 23:8859-8866.
58. Muslimov I A, Nimmrich V, Hernandez A I, Tcherepanov A, Sacktor T C, Tiedge H (2004) Dendritic transport and localization of protein kinase Mζ mRNA: implications for molecular memory consolidation. J Biol Chem 279:52613-52622.
59. Schratt G M, Tuebing F, Nigh E A, Kane C G, Sabatini M E, Kiebler M, Greenberg M E (2006) A brain-specific microRNA regulates dendritic spine development. Nature 439:283-289.
60. Tubing F, Vendra G, Mikl M, Macchi P, Thomas S, Kiebler M A (2010) Dendritically localized transcripts are sorted into distinct ribonucleoprotein particles that display fast directional motility along dendrites of hippocampal neurons. J Neurosci 30:4160-4170.
61. Doyle M, Kiebler M A (2011) Mechanisms of dendritic mRNA transport and its role in synaptic tagging. EMBO J 30:3540-3552.
62. Robeck T, Skryabin B V, Rozhdestvensky T S, Skryabin A B, Brosius J (2016) BC1 RNA motifs required for dendritic transport in vivo. Sci Rep 6:28300.
63. Muslimov I A, Titmus M, Koenig E, Tiedge H (2002) Transport of neuronal BC1 RNA in Mauthner axons. J Neurosci 22:4293-4301.
64. Congdon E E, Gu J, Sait H B, Sigurdsson E M (2013) Antibody uptake into neurons occurs primarily via clathrin-dependent Fc7 receptor endocytosis and is a prerequisite for acute tau protein clearance. J Biol Chem 288:35452-35465.
65. Douglas J N, Gardner L A, Levin M C (2013) Antibodies to an intracellular antigen penetrate neuronal cells and cause deleterious effects. J Clin Cell Immunol 4:1-7.
66. Kazim S F, Chuang S C, Zhao W, Wong R K, Bianchi R, Iqbal K (2017) Early-onset network hyperexcitability in presymptomatic Alzheimer's disease transgenic mice is suppressed by passive immunization with anti-human APP/Abeta antibody and by mGluR5 blockade. Front Aging Neurosci 9:71.
67. Rhodes D A, Isenberg D A (2017) TRIM21 and the function of antibodies inside cells. Trends Immunol 38:916-926.
68. Kowal C, Degiorgio L A, Lee J Y, Edgar M A, Huerta P T, Volpe B T, Diamond B (2006) Human lupus autoantibodies against NMDA receptors mediate cognitive impairment. Proc Natl Acad Sci USA 103:19854-19859.
69. Muslimov I A, Banker G, Brosius J, Tiedge H (1998) Activity-dependent regulation of dendritic BC1 RNA in hippocampal neurons in culture. J Cell Biol 141:1601-1611.
70. Schoch A, Thorey I S, Engert J, Winter G, Emrich T (2014) Comparison of the lateral tail vein and the retro-orbital venous sinus routes of antibody administration in pharmacokinetic studies. Lab Anim 43:95-99.
71. Nimmerjahn F, Ravetch J V (2008) Fc7 receptors as regulators of immune responses. Nat Rev Immunol 8:34-47.
72. Doens D, Fernandez P L (2014) Microglia receptors and their implications in the response to amyloid beta for Alzheimer's disease pathogenesis. J Neuroinflammation 11:48.
73. Tiedge H, Brosius J (1996) Translational machinery in dendrites of hippocampal neurons in culture. J Neurosci 16:7171-7181.
74. Muddashetty R S, Kelic S, Gross C, Xu M, Bassell G J (2007) Dysregulated metabotropic glutamate receptor-dependent translation of AMPA receptor and postsynaptic density-95 mRNAs at synapses in a mouse model of fragile X syndrome. J Neurosci 27:5338-5348.
75. Lin Y, Brosius J, Tiedge H (2001) Neuronal BC1 RNA: co-expression with growth-associated protein-43 messenger RNA. Neuroscience 103:465-479.
76. Jain C, Belasco J G (1996) A structural model for the HIV-1 Rev-RRE complex deduced from altered-specificity Rev variants isolated by a rapid genetic strategy. Cell 87:115-125.
77. Rangan R, Zheludev I N, Hagey R J, Pham E A, Wayment-Steele H K, Glenn J S, Das R (2020) RNA genome conservation and secondary structure in SARS-CoV-2 and SARS-related viruses: a first look. RNA 26:937-959.
78. Wharton R P (2009) A splicer that represses (translation). Genes Dev 23:133-137.
79. Gao Y, Tatavarty V, Korza G, Levin M K, Carson J H (2008) Multiplexed dendritic targeting of a calcium calmodulin-dependent protein kinase II, neurogranin, and activity-regulated cytoskeleton-associated protein RNAs by the A2 pathway. Mol Biol Cell 19:2311-2327.
80. Rozhdestvensky T S, Crain P F, Brosius J (2007) Isolation and posttranscriptional modification analysis of native BC1 RNA from mouse brain. RNA Biol 4:11-15.
81. Kowal C, DeGiorgio L A, Nakaoka T, Hetherington H, Huerta P T, Diamond B, Volpe B T (2004) Cognition and immunity; antibody impairs memory. Immunity 21:179-188.
82. Yardeni T, Eckhaus M, Morris H D, Huizing M, Hoogstraten-Miller S (2011) Retro-orbital injections in mice. Lab Anim 40:155-160.
83. Skryabin B V, Sukonina V, Jordan U, Lewejohann L, Sachser N, Muslimov I, Tiedge H, Brosius J (2003) Neuronal untranslated BC1 RNA: targeted gene elimination in mice. Mol Cell Biol 23:6435-6441.
84. Tiedge H, Lünsdorf H, Schäfer G, Schairer H U (1985) Subunit stoichiometry and juxtaposition of the photosynthetic coupling factor 1: Immunoelectron microscopy using monoclonal antibodies. Proc Natl Acad Sci USA 82:7874-7878.
85. Muddashetty R, Khanam T, Kondrashov A, Bundman M, Iacoangeli A, Kremerskothen J, Duning K, Barnekow A, Huttenhofer A, Tiedge H, Brosius J (2002) Poly(A)-binding protein is associated with neuronal BC1 and BC200 ribonucleoprotein particles. J Mol Biol 321:433-445.
86. Kremerskothen J, Zopf D, Walter P, Cheng J G, Nettermann M, Niewerth U, Maraia R J, Brosius J (1998) Heterodimer SRP9/14 is an integral part of the neural BC200 RNP in primate brain. Neurosci Lett 245:123-126.
87. Bovia F, Wolff N, Ryser S, Strub K (1997) The SRP9/14 subunit of the human signal recognition particle binds to a variety of Alu-like RNAs and with higher affinity than its mouse homolog. Nucleic Acids Res 25:318-326.
88. Colino J, Duke L, Snapper C M (2013) Noncovalent association of protein and capsular polysaccharide on bacteria-sized latex beads as a model for polysaccharide-specific humoral immunity to intact gram-positive extracellular bacteria. J Immunol 191:3254-3263.
89. Brown V J, Bowman E M (2002) Rodent models of prefrontal cortical function. Trends Neurosci 25:340-343.
90. Tait D S, Chase E A, Brown V J (2014) Attentional set-shifting in rodents: a review of behavioural methods and pharmacological results. Curr Pharm Des 20:5046-5059.
91. Garner J P, Thogerson C M, Wurbel H, Murray J D, Mench J A (2006) Animal neuropsychology: validation of the Intra-Dimensional Extra-Dimensional set shifting task for mice. Behav Brain Res 173:53-61.
92. Farrell J S, Wolff M D, Teskey G C (2017) Neurodegeneration and pathology in epilepsy: clinical and basic perspectives. Adv Neurobiol 15:317-334.
93. Iacoangeli A, Lin Y, Morley E J, Muslimov I A, Bianchi R, Reilly J, Weedon J, Diallo R, Böcker W, Tiedge H (2004) BC200 RNA in invasive and preinvasive breast cancer. Carcinogenesis 25:2125-2133.
94. Nimmrich V, Hargreaves E L, Muslimov I A, Bianchi R, Tiedge H (2005) Dendritic B C1 RNA: modulation by kindling-induced after discharges. Brain Res Mol Brain Res 133:110-118.
95. Krienke C, Kolb L, Diken E, Streuber M, Kirchhoff S, Bukur T, Akilli-Ozturk O, Kranz L M, Berger H, Petschenka J, Diken M, Kreiter S, Yogev N, Waisman A, Kariko K, Tureci O, Sahin U (2021) A noninflammatory mRNA vaccine for treatment of experimental autoimmune encephalomyelitis. Science 371:145-153.
96. Chi J M, Mackay M, Hoang A, Cheng K, Aranow C, Ivanidze J, Volpe B, Diamond B, Sanelli P C (2019) Alterations in blood-brain barrier permeability in patients with systemic lupus erythematosus. AJNR Am J Neuroradiol 40:470-477.
97. Soybilgic A (2015) Neuropsychiatric systemic lupus erythematosus in children. Pediatr Ann 44:e153-158.
98. Devinsky O, Schein A, Najjar S (2013) Epilepsy associated with systemic autoimmune disorders. Epilepsy Curr 13:62-68.
99. Steinlin M I, Blaser S I, Gilday D L, Eddy A A, Logan W J, Laxer R M, Silverman E D (1995) Neurologic manifestations of pediatric systemic lupus erythematosus. Pediatr Neurol 13:191-197.
100. Lazaro D (2007) Elderly-onset systemic lupus erythematosus: prevalence, clinical course and treatment. Drugs Aging 24:701-715.
101. Pons-Estel G J, Alarcon G S, Scofield L, Reinlib L, Cooper G S (2010) Understanding the epidemiology and progression of systemic lupus erythematosus. Semin Arthritis Rheum 39:257-268.
102. Lewis M J, Jawad A S (2017) The effect of ethnicity and genetic ancestry on the epidemiology, clinical features and outcome of systemic lupus erythematosus. Rheumatology (Oxford) 56:i67-i77.
103. Maningding E, Dall'Era M, Trupin L, Murphy L B, Yazdany J (2020) Racial and ethnic differences in the prevalence and time to onset of manifestations of systemic lupus erythematosus: the California Lupus Surveillance Project. Arthritis Care Res 72:622-629

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A composition for treating neuropsychiatric lupus comprising a BC RNA200 decoy of SEQ ID NO: 7, said decoy inhibiting SLE-anti-BC IgG mediated displacement of one or more of transport factors, hnRNP A2 and Purα, from BCRNA dendritic targeting elements.

2. A composition for treating neuropsychiatric lupus comprising a BC RNA200 decoy of SEQ ID NO: 8, said decoy inhibiting SLE-anti-BC IgG mediated displacement of one or more of transport factors, hnRNP A2 and Purα, from BCRNA dendritic targeting elements.

3. The composition of claim 1, wherein said sequence comprises a terminal phosphate at the 5′ end, at the 3′end or at both ends of said sequence, thereby inhibiting exonuclease degradation of said decoy sequence.

4. The composition of claim 1, comprising an additional 1, 2, 3, 4, 5, 6, 7, 8, 9, 10, 20 or 30 nucleotides at the 3′ end or the 5′end of said sequence, said additional nucleotides being complementary to nucleotides present in the BCRNA200 region bound by said BC RNA200 decoy.

5. The composition of claim 1, comprising at least one modified nucleotide, wherein said modification is not present in nucleotides which hybridize to bulge region of the decoy.

6. The composition of claim 1, wherein said modification is selected from one or more of 2-O-methyl, 2-O-methyoxy, LNA, FANA, UNA, a peptide nucleic acid, and an unnatural nucleotide.

7. The composition claim 1, where at least one phosphodiester linkage is substituted with an internucleotide linkage selected from a phosphorothioate linkage, dithioate, 1-12C alkylphosphonate, methylphosphonate, amidate and triester.

8. The composition claim 1, further comprising a cytoplasmic delivery vehicle.

9. The composition of claim 8, wherein the cytoplasmic delivery vehicle is selected from the group consisting of a liposome, synthetic polymer, cell-penetrating peptide, nanoparticle, viral particle, electroporation buffer, and nucleofection reagent.

10. The composition claim 1, wherein said BC200 decoy is bound via a spacer to a nanoparticle.

11. The composition of claim 10, wherein said nanoparticle is a lipid nanoparticle.

12. The composition of claim 10, wherein said spacer is an organic moiety.

13. The composition of claim 12, wherein said organic moiety is a polymer.

14. The composition of claim 13, wherein said polymer is a water soluble polymer, a nucleic acid, a polypeptide or an oligosaccharide.

15. The composition of claim 1, wherein said decoy sequence present in a vector.

16. The composition of claim 15, wherein said vector is a viral vector.

17. The composition of claim 16, wherein said vector is selected from an adenoviral vector, an adeno-associated viral vector, a retroviral vector, and a lentiviral vector.

18. The composition of claim 17, wherein said viral vector is an adeno-associated vector.

19. The composition of claim 1, further comprising at least one of an anti-inflammatory drug, an anticoagulation agent, and an immunosuppressive agent.

20. The composition of claim 1, further comprising a pharmaceutically acceptable carrier.

21. A method for treating neuropsychiatric lupus in a patient in need thereof comprising administration of an effective amount of the composition of claim 1, to a patient in need thereof.
